(19) United States
(12) Patent Application Publication
(10) Pub. No.: US 2024/0352424 A1
(43) Pub. Date: Oct. 24, 2024

(54) METHODS OF PRODUCING TENDON NEOTISSUE FROM ADULT STEM CELLS AND USES THEREOF

(71) Applicant: Board of Supervisors of Louisiana State University and Agricultural and Mechanical College, Baton Rouge, LA (US)

(72) Inventors: Mandi J. LOPEZ, Saint Gabriel, LA (US); Takashi TAGUCHI, Baton Rouge, LA (US)

(73) Assignee: Board of Supervisors of Louisiana State University and Agricultural and Mechanical College, Baton Rouge, LA (US)

(21) Appl. No.: 18/642,228

(22) Filed: Apr. 22, 2024

Related U.S. Application Data
(60) Provisional application No. 63/461,109, filed on Apr. 21, 2023.

(51) Int. Cl. C12N 5/077; A61L 27/36; C12M 1/00; C12M 1/42; C12M 3/00; C12N 5/00

(52) U.S. Cl. CPC C12N 5/066; A61L 27/3637; C12M 21/08; C12M 29/10; C12M 35/04; C12N 5/0068; C12N 2501/15; C12N 2506/1384; C12N 2513/00

(57) ABSTRACT

The invention relates to a method of producing a tendon neotissue from a population of adipose-derived stem cells (ASCs) subjected to mechanical and biological stimulations in a bioreactor system. The tendon neotissues are effective as implants to treat tendon or ligament injury in a subject. The invention also relates to a customized bioreactor useful for producing a tri-dimensional engineered tissue.

### RELATED APPLICATION

This application claims priority to U.S. Provisional Application No. 63/461,109, filed Apr. 21, 2023, the contents of which are incorporated herein by reference in their entirety.

### SEQUENCE LISTING

The instant application contains a Sequence Listing XML which has been submitted electronically in EFS and is hereby incorporated by reference in its entirety. Said XML copy, created on Apr. 4, 2024, is named 144240591412 and is 38,122 bytes in size.

### BACKGROUND

### 1. Technical Field

Currently claimed embodiments of the invention relate to de novo tendon neotissue, methods of inducing tenogenic differentiation in a population of stem cells, methods of producing tendon neotissues and their applications for cell-based therapy to repair tendon or ligament-associated injuries including tendinopathy and desmitis. Claimed embodiments of the invention also relate to novel bioreactor systems for producing tri-dimensional engineered tissues from undifferentiated cells, including multipotent stem cells.

### 2. Discussion of Related Art

Tendinopathy and desmitis comprise a large majority of musculoskeletal injuries that are responsible for up to 72% of lost training days and 14% of early retirements by equine athletes. Superficial digital flexor tendinopathy and suspensory ligament (SL) desmitis are the most common, comprising 46% of all limb injuries. The predominant type of tendon and ligament injury varies among disciplines, but all equine companions can be impacted. Strain induced injuries are common in the equine suspensory apparatus including the suspensory ligament, superficial digital flexor tendon, and deep digital flexor tendon. Many acute and chronic tendon and ligament lesions are thought to result from focal accumulation of microtrauma and poorly organized repair tissue that can coalesce into large lesions and predispose to spontaneous rupture in many species.

Diagnosis is usually a combination of physical examination and ultrasound imaging. Treatments vary widely and can range from rest with anti-inflammatory drugs, cold therapy and pressure bandaging to intralesional injections of various therapeutics and extracorporeal shock wave therapy. Intralesional regenerative therapies such as platelet rich plasma, stem cells, and genetic material have been applied with variable success. Poor or abnormal tissue repair contributes to a reinjury rate in horses as high as 67% within 2 years. To date, there is no single, gold standard to promote healing of ligament and tendon lesions.

Strain induced injuries are common in the equine suspensory apparatus including the SL, SDFT, and deep digital flexor tendon (DDFT). Low cell numbers and metabolic activity, limited blood supply, and failure of endogenous tenocytes and ligamentocytes to migrate to the injury site contribute to poor tissue healing capacity.

There are four recognized stages of tendon and ligament healing: an acute inflammatory phase, a subacute reparative phase, a collagen phase, and a chronic remodeling phase. Low cell numbers and metabolic activity, limited blood supply, and failure of endogenous tenocytes and ligamentocytes to migrate to the injury site affect all stages of healing and contribute to poor tissue healing capacity in adult amilas. However, recent research confirms enhanced healing capacity of neonatal tendon over that of adults owing to migration of endogenous tenocytes recruited by TGF-β signaling to the site of injury and replacing early fibrous scar tissue with normal tendon. The tenocytes replace early fibrous scar tissue with normal tendon. Based on existing knowledge, the optimum time to deliver exogenous cells to an injured tendon or ligament horses is around the sixth day after injury at the transition between the inflammatory and subacute reparative phases.

Autologous tenocyte implantation is one mechanism to deliver endogenous cells to the site of tendon or ligament injury in adult animals and humans. However, the therapy is limited by few harvest sites and harvest morbidity, and it is not practical in horses. Administration of exogenous adult multipotent stromal cells (MSCs) is reported to augment natural healing in naturally occurring and experimentally induced equine tendon and ligament injuries. Results are mixed, in part due to differences among cell isolates, lesions, individual healing capacity, and low engraftment of exogenous cells (<0.001%). Further, there is evidence that an inflammatory environment may impede differentiation of MSCs, and the cells may assume an abnormal phenotype leading to unwanted side effects.

Implantation of tendon neotissues produced from the differentiation of multipotent stem cells such as adipose-derived stem cells (ASCs) is a promising approach to tendon or ligament repair. Neotissue generated from ASCs is likely to have value for both tendon and ligament lesions and has the potential to augment therapies for equine tendinopathy. There is an unmet need to develop a reliable method of producing de novo tendon tissue or neotissue derived from multipotent stem cells that overcomes the current limitations.

### SUMMARY OF THE INVENTION

A method of inducing tenogenic differentiation in a population of adipose-derived stem cells (ASCs) cultured in a bioreactor system, includes: directly applying or infusing said population of ASCs onto a porous biopolymer-based scaffold to form a cell-scaffold construct, culturing said cell-scaffold construct in a medium for a period of time to produce a population of tenocyte-like cells, which includes: contacting said cell-scaffold construct with at least one tenogenic differentiation driver, and applying controlled mechanical stimulations to said cell-scaffold construct comprising: flow shear stress, and dynamic or static tensile strain, to produce a population of differentiated cells, expressing at least one tenogenic transcription factor, at least one tendon-specific extracellular matrix gene, or a combination thereof, maturing said population of differentiated cells to produce a population of tenocyte-like cells, wherein said population of tenocyte-like cells express at least one tendon marker gene.

The method of the preceding embodiment, where the population of ASCs is a population of equine adult adipose-derived multipotent stem cells.

The method of the preceding embodiment, where the population of differentiated cells comprises a population of tenoblast-like cells.

A method of producing a tendon neotissue from a population of ASCs cultured in a bioreactor system, includes: directly applying or infusing said population of ASCs onto a porous biopolymer-based scaffold to form a cell-scaffold construct, culturing said cell-scaffold construct in a medium for a period of time to produce a population of tenocyte-like cells, which includes: contacting said cell-scaffold construct with at least one tenogenic differentiation driver, and applying controlled mechanical stimulations to said cell-scaffold construct comprising: flow shear stress, and dynamic or static tensile strain, wherein said population of tenocyte-like cells express at least one tendon marker gene and organize to form said tendon neotissue.

The method of the preceding embodiment, where the tendon neotissue further comprises a population of tenoblast-like cells.

The method of any one of the preceding embodiments, where the porous biopolymer-based scaffold is ligated by a filament or a net and shaped as a column along a longitudinal axis.

The method of the preceding embodiment, where the tendon neotissue comprises the population of tenocyte-like cells are embedded within a fibrous extracellular matrix (ECM) attached to the biopolymer-based scaffold and are organized parallel to each other along the biopolymer-based scaffold longitudinal axis.

The method of any of the preceding embodiments, where the population of tenocyte-like cells have an elongated rod-like nucleus and express ECM components.

The method of any of the preceding embodiments, where said flow shear stress is induced by a perfusion flow and a centrifugal flow motion of the medium.

The method of any of the preceding embodiments, where the at least one tenogenic differentiation driver is a member of the TGF-β growth factor family, preferably TGF-β1.

An implant comprising at least one tendon neotissue produced according to the method of the preceding embodiments, said implant being effective for use in the treatment of a tendon or ligament injury in a mammal subject.

The implant according to the preceding embodiment, further comprises a molecule of the group consisting of collagen, laminin, fibronectin, PLA, PGLA, PLLA, PEEK, PEG, elastin, tenomodulin, fibromodulin, and any combination thereof.

A method of treating a tendon or ligament injury (e.g., tendinopathy and desmitis) comprising the implantation of at least one tendon neotissue produced according to the method of any of the preceding embodiments, or at least one implant according to the preceding embodiments, in a mammal subject.

A bioreactor system for producing a tri-dimensional engineered tissue includes: a bioreactor including: a base chamber containing a medium and a construct comprising a biopolymer-based scaffold comprising a population of multipotent cells, a core frame comprising an immobile horizontal bar at the bottom for securing one extremity of said construct and a gas exchange access port, a top lid comprising: a medium access port, and an adjustable horizontal bar for securing the other extremity of said construct, said adjustable horizontal bar being attached to a vertical threaded bar that can move upward and downward thereby allowing for a change in distance between said immobile horizontal bar and said adjustable horizontal bar to apply an adjustable dynamic or static tensile strain to said construct. A perfusion system includes a medium reservoir containing the medium, a deformable membrane for facilitating gas exchange with said base chamber, a medium access port and a gas exchange access port, a peristaltic pump connected to said medium access port of said medium reservoir and to said medium access port of said top lid for controlling the rate and directionality of a flow of said medium in and/or out of said base chamber, an agitator to create centrifugal flow motion and enhance nutrients and gas diffusion.

The bioreactor system of any of the preceding embodiments, where said perfusion system produces a bidirectional perfusion flow and a centrifugal flow motion, thereby inducing flow shear stress onto the construct.

The bioreactor system of any of the preceding embodiments, where the biopolymer-based scaffold comprises collagen of type I and the population of multipotent cells is a population of ASCs.

The bioreactor system of any of the preceding embodiments, where the flow of medium comprises at least one tenogenic differentiation driver.

### DESCRIPTION OF THE FIGURES

FIG. 1 shows the study design of ASCs isolation to the culture onto COLI scaffold in a bioreactor system.

FIGS. 2A-2D show ligated COLI scaffold (A) and immobile horizontal bar located inside core frame and adjustable horizontal bar on which the scaffold is attached inside the bioreactor chamber.

FIGS. 3A-3D illustrate the bioreactor (FIG. 3A), a cross-section of the bioreactor (FIG. 3B), an exploded view of the bioreactor (FIG. 3C) and a perspective view of the bioreactor (FIG. 3D).

FIGS. 4A-4B are images of a bioreactor system according to embodiments for culturing cell-scaffold constructs under static strain (A), and cyclic tensioner under dynamic strain (B).

FIG. 5 is a schematic representation of COLI construct specimen harvest scheme.

FIGS. 6A-6F are images of COLI constructs cultured in stromal medium (FIGS. 6A-6C) or tenogenic medium (FIGS. 6D-6F) for 7 (FIGS. 6A and 6D), 14 (FIGS. 6B and 6E), and 21 (FIGS. 6C and 6F) days; the upper area of each image corresponds to the top of the construct in its longitudinal axis.

FIG. 7 provides confocal microscopy images showing the viable cell morphology and distribution in COLI constructs under static strain. Constructs were cultured in stromal medium (top row) or tenogenic medium (bottom row) for 7 (left column), 14 (middle column), or 21 (right column) days.

FIG. 8 is a graph showing the relative viable cell number in constructs under static strain after culture in stromal or tenogenic medium. A difference between culture medium columns is indicated by an asterisk (p<0.05).

FIGS. 9A-9C are graphs showing tendon-specific gene expression of cells in constructs cultured under static strain; constructs were cultured in stromal or tenogenic medium for 7, 14, or 21 days, and gene expression of cells cultured in tenogenic medium was normalized to that of stromal medium and expressed as fold change at each culture period; expression of tenogenic transcription factors, Scx, Mkx, Egr1, CTGF, and LOX (FIG. 9A); expression of tenogenic ECM genes, Col1a1, Col3a1, Dcn, Eln, TnC, and Bgn (FIG. 9B); expression of mature tendon markers, Fbmd, Col14a1, and THBS4 (FIG. 9C). Asterisks indicate a difference from 1-fold within each culture period.

FIGS. 10A-10F show histological microstructure of constructs cultured in stromal (FIGS. 10A, 10C, and 10E) or tenogenic (FIGS. 10B, 10D, and 10F) medium for 7 (FIGS. 10A and 10B), 14 (FIGS. 10C and 10D), and 21 (FIGS. 10E and 10F) days; Scale bars=50 μm (FIGS. 10A-10C, and 10E) and 100 μm (FIG. 10D and FIG. 10F). Cells (arrows) within variable amounts of ECM as well as template material are apparent.

FIG. 11 show fibromodulin (Fbmd) localization in COLI-ASC constructs cultured in stromal (left) or tenogenic (right) medium for 7, 14, or 21 days, and in a section of deep digital flexor tendon (DDFT). A construct section stained with only secondary antibody (2° Ab only) is also shown. DAPI counterstain; Scale bar=100 μm.

FIGS. 12A-12F show ultrastructure of constructs cultured in stromal (FIGS. 12A-12C) or tenogenic (FIGS. 12D-12F) medium for 7 (FIGS. 12A and 12D), 14 (FIGS. 12B and 12 E), and 21 (FIGS. 12C and 12F) days obtained by scanning electron microscopy.

FIGS. 13A-13B shows electron microphotographs of cells from ASC-COLI constructs cultured in stromal (A) or tenogenic (B) medium for 21 days with fibrils in the ECM surrounding the latter (arrows).

FIGS. 14A-14B show histological microstructure of constructs cultured in tenogenic for 21 days under dynamic strain; white inset in FIG. 14A is enlarged in FIG. 14B.

FIGS. 15A-15C show ultrastructure of constructs cultured in tenogenic medium for 21 days under dynamic strain. Cells (white arrows) were found surrounded by newly deposited ECM (cross-hatched arrows) on collagen fibers of original template (grey arrows); inset square in FIG. 15B is enlarged in FIG. 15C.

FIG. 16 shows the study design of SD (Sprague Dawley) rat bilateral calcancal tendon elongation injury model creation and neotissue treatment.

FIG. 17 shows study design of RNU (Rowett Nude) rat bilateral calcancal tendon elongation injury model creation and neotissue treatment.

FIGS. 18A-18L illustrate images of elongation-induced calcaneal tendinopathy model creation.

FIGS. 19A-19D illustrate implantation of ASC COLI construct into rat calcancal tendon; a cylindrical piece of neotissue (4 mm diameter, 10 mm thickness) placed into the tip of 20-gauge 1 inch needle was inserted into the calcaneal tendon transdermally from the distal (FIG. 19A) to proximal (FIG. 19B) end of the tendon; a metal stylet from a 22-gauge 1 inch intravenous catheter was inserted into a 20-gauge hypodermic needle (FIG. 19C) and advanced while withdrawing needle (FIG. 19D) to implant the construct

FIGS. 20A-20B show graphs of functional score of hind limbs in SD (FIG. 20A) and RNU (FIG. 20B) rats from pre-injury to 6 weeks post-injury; functional scores were obtained for limbs that received tenogenic constructs (circle), stromal constructs (square), or PBS (triangle). Each treatment was administered 6 days after injury.

FIGS. 21A-21B show graphs of surgical percent tendon elongation (A) and tendon width at harvest (B) of SD rats.

FIGS. 22A-22B show graphs of surgical percent tendon elongation (A) and tendon width at harvest (B) of RNU rats.

FIGS. 23A-23F show images of photomicrographs of an elongated calcaneal tendon treated with tenogenic construct in SD rats; harvested calcaneal tendon was stained with hematoxylin and eosin (FIGS. 23A, 23C, 23E) or Masson's trichrome (FIGS. 23B, 23D, 23F); the implant is indicated by a black dotted line.

FIGS. 24A-24F show photomicrographs of an elongated calcaneal tendon treated with a stromal construct in a SD rat; harvested calcancal tendon was stained with hematoxylin and eosin (A, C, E) or Masson's trichrome (B, D, F). The implant is indicated by a black dotted line.

FIGS. 25A-25F show photomicrographs of an elongated calcaneal tendon treated with PBS in a SD rat; harvested calcaneal tendon was stained with hematoxylin and eosin (A, C, E) or Masson's trichrome (B, D, F). The implant is indicated by a black dotted line.

FIGS. 26A-26F show photomicrographs of an elongated calcaneal tendon treated with a tenogenic construct in an RNU rat; harvested calcaneal tendon was stained with hematoxylin and eosin (A, C, E) or Masson's trichrome (B, D, F). The implant is indicated by a black dotted line.

FIGS. 27A-27F show photomicrographs of an elongated calcaneal tendon treated with a stromal construct in an RNU rat; harvested calcaneal tendon was stained with hematoxylin and eosin (A, C, E) or Masson's trichrome (B, D, F). The implant is indicated by a black dotted linc.

FIGS. 28A-28F show photomicrographs of an elongated calcaneal tendon treated with PBS in an RNU rat; harvested calcaneal tendon was stained with hematoxylin and eosin (A, C, E) or Masson's trichrome (B, D, F). The implant is indicated by a black dotted line.

FIG. 29 shows graphs showing tendon-specific gene expression of cells in injured calcancal tendon tissue of SD rats ˜5 weeks after treatment with constructs cultured in stromal medium or ˜5 weeks after treatment with PBS. Gene expression of tissues treated with constructs cultured in stromal medium or tissues treated with PBS was normalized to that of tissues treated with constructs cultured in tenogenic medium and expressed as fold change.

### DETAILED DESCRIPTION

All publications and patent applications identified herein are incorporated by reference in their entirety.

Some embodiments of the current subject matter are discussed in detail below. In describing embodiments, specific terminology is employed for the sake of clarity. However, the embodiments are not intended to be limited to the specific terminology so selected. A person skilled in the relevant art will recognize that other equivalent components can be employed, and other methods developed, without departing from the broad concepts of the present subject matter. All references cited anywhere in this specification are incorporated by reference as if each had been individually incorporated.

Unless defined otherwise, technical, and scientific terms used herein have the same meaning as commonly understood by a person of ordinary skill in the art. The following definitions are provided to facilitate understanding of certain terms used frequently herein and are not meant to limit the scope of the present disclosure.

As used throughout, the term “differentiation” refers to a process by which an undifferentiated cell, such as a pluripotent or a multipotent cell, including but not limited to a multipotent stromal stem cell (MSC) such as an adipose-derived stem cell (ASC), adopts a certain fate. The term “maturation” refers to a process by which a cell which has already undergone differentiation (committed cell fate) becomes more specialized over time and achieves its specific functions. In some embodiments, the matured cell exhibits a tenocyte-like phenotype. In certain embodiments, the differentiation process of multipotent cells gives rise to two or more differentiated cell populations. For example, in vitro differentiation of a population of ASCs may be prodded under biochemical and/or mechanical cues into becoming mixed population of cells at various stages of differentiation, which give rise to tendon-specific lineage including tenoblasts, tenocytes and ligamentocytes.

As used throughout, the term “tenogenic differentiation” refers to a process by which an undifferentiated cell, including but not limited to an ASC, transitions from an undifferentiated cell type to a differentiated cell. For example, the differentiated cells may overexpression tenogenic transcription factor and/or tendon-specific ECM genes and/or exhibit a tenoblast-like phenotype. In some embodiments, differentiated cells can further mature and exhibit a tenocyte-like phenotype. In the tenogenic process of some embodiments, differentiation and/or maturation of cells rely on biochemical and/or mechanical stimulations.

As used throughout, the term “a population of stem cells” refers to a population of pluripotent or multipotent cells defined by their capacity for self-renewal and for producing lineages capable of differentiating into one or more specialized cell types. Stem cells can be found in developing embryos, in the stroma of various adult tissues such as the stroma or can be engineered from somatic cells. In regenerative medicine, stem cells are critical for tissues repair.

As used throughout, the term “adipose-derived stem cells” population refers to multipotent cells, which have a capacity for self-renewal and can differentiated into various cell lineages such as tenocytes, adipocytes, chondrocytes, myocytes, and neurocytes among others. They can be harvested by direct excision or other techniques well-known in the art. In some embodiments, ASCs may be isolated from adipose-tissue after digestion with collagenase or any other suitable enzyme and maintained in a basic stroma medium (DMEM-Ham's F12, 10% FBS, 1% antibiotic/antimycotic) or stored by cryopreservation. During the tenogenic differentiation process, ASCs may be cultured in a tenogenic medium supplemented with a tenogenic differentiation driver such as transforming growth factor (TGF)-β1.

As used throughout, the term “tenocytes” or “tenocyte-like cells” refers to differentiated specialized cells which are responsible for synthesis, build up and turnover of tendon fibers and tendon-specific ECM components. They are elongated fibroblast-like cells and are typically arranged parallel to each other in the longitudinal direction of the tendon. The tenocyte's cytoplasm stretches between the collagen fibers of the tendon and forms thin cytoplasmatic projections allowing cell-cell interactions via gap junctions. The morphology and function of the tenocytes may be maintained under biochemical or mechanical stimulations.

As used throughout, the term “tenoblasts” or “tenoblast-like cells” refers to ovoid cells with large nuclei. They can differentiate into tenocytes and are involved in the tendon healing process. Tenoblast-like cells express tenogenic genes, including tendon-specific ECM genes to produce tendon-like extracellular matrix.

As used throughout, the term “contacting a cell population” refers to placing a molecule in proximity to the cells. In certain embodiments, the molecule is placed in the environment of a population of cells to affect their metabolism, function(s), differentiation status, maturation, proliferation, or growth. In certain embodiments, the molecule may be an inducer, an enhancer, a driver of tenogenic differentiation, tenogenic maturation and/or cell growth. The tenogenic differentiation driver may be added to a culture medium, may already be formulated in the culture medium, can be produced by a cell population in co-culture or may be infused onto the scaffold which serves as support for the differentiation process of the stromal stem cells.

As used throughout, the term “tenogenic differentiation driver” refers to a molecule that can induce, activate and/or enhance differentiation of multipotent cells towards specialized cells. In certain embodiments, tenogenic differentiation drivers induce, activate and/or enhance the differentiation of multipotent ASCs towards differentiated cells of the tenogenic fate including, but not limited to, tenoblasts-like cells, tenocytes-likes cells or ligamentocytes. Tenogenic differentiation drivers may also be used for the maturation of differentiated cells. In some embodiments, the tenogenic drivers include specific scaffold biopolymers which can mimic ECM components as well as growth factors, particularly transforming growth factor beta (TGF-β), preferably TGF-β 1. The tenogenic differentiation drivers may supplement the culture medium or/and may be present in the scaffold used as a support for cell growth, proliferation, and differentiation.

As used throughout, the term “tenogenic transcription factors” refers to molecules that initiate, enhance and/or regulate the tenogenic differentiation process. Such transcription factors upregulate the expression of various genes involved in the differentiation process, the production of ECM and/or the production of tendon-specific protein such as fibromodulin and tenomodulin. Example of tenogenic transcription factors include but are not limited to Sex, Mkx, Egr1, CTGF, and LOX. In certain embodiments, Scx, a relatively specific marker of tendon/ligament lineage initiates the differentiation process of the ASCs under biomechanical stimulations. Mkx and Egr1 have been reported to be involved in tendon formation.

As used throughout, the term “marker” refers to a gene or its protein product expressed by a specific type of cells which can be used to characterize, identify, or isolate the cells. In some embodiments, tendon-specific markers are characteristic of a cell population sharing the same function and having a tenocyte-like phenotype. In other embodiments, tendon-specific ECM markers can be expressed by cells of different type such as cells having a tenoblast-like phenotype and cells having a tenocyte-like phenotype.

As used throughout, the term “ECM” refers to tendon-specific extracellular matrix or at least one component or a mixture of selected component of the tendon-specific extracellular matrix that embed the tendon cells. In the tendon, the ECM functions as the organizer for collagen fibril assembly, and merely comprises proteoglycan, glycoproteins, and other small molecules. Decorin (Den) and biglycan (Bgn) are small leucine-rich proteoglycans that help organize neotissue fibers. Other common proteoglycans are fibromodulin and lumican. tenascin-C (TnC), a glycoprotein, is regulated by mechanical loading and is upregulated in patients with tendinopathy. Moreover, TnC also participates in collagen fiber alignment and orientation. Tenomodulin (Tnmd) is a type II transmembrane glycoprotein containing a C-terminal antiangiogenic domain, and it is necessary for tenocyte proliferation and tendon maturation.

As used throughout, the term “expanding a cell population” refers to increasing the number of cells within a cell population.

As used throughout, the term “tendon neotissue” refers to a tissue that mimics the physiology and mechanical properties of native immature tendon tissue. The neotissue comprises tenocyte-likes and/or tenoblast-like cells embedded into aligned collagen-rich fibers and ECM materials. Most fibers are aligned parallel to each other and allow the neotissue to sustain loads in one direction and provides a high tensile strength. In the body, tendons exist between muscle and bone. Their primary function is to transmit force and stabilize joints. Tendons resist tensile forces.

### Methods of Inducing Tenogenic Differentiation in a Population of Multipotent Stem Cells

Certain embodiments relate to methods of inducing tenogenic differentiation in a population of undifferentiated cells, including but not limited to multipotent stem cells (MSCs) such as adipose-derived stem cells (ASCs).

In certain embodiments, a population of ASCs is cultured in a bioreactor system which allows the circulation of a flow of culture medium. In some embodiments, the culture medium is a stromal medium and comprises basic nutrients facilitating the growth and/or proliferation of the cells in culture. For example, a formulation of a stromal medium may comprise DMEM-Ham's F12, 10% FBS, 1% antibiotic/antimycotic. In other embodiments, the culture medium is a tenogenic medium which facilitates the tenogenic differentiation of stem cells and comprises a stromal medium supplemented with at least one tenogenic differentiation driver. For example, a formulation of a tenogenic medium may be supplemented with at least one of the ingredients selecting from the group of 1% FBS, 10 ng/mL transforming growth factor (TGF)-β1, 50 μM/L-ascorbic acid 2-phosphate sesquimagnesium salt hydrate, 50 μg/mL insulin and 1% antibiotic/antimycotic.

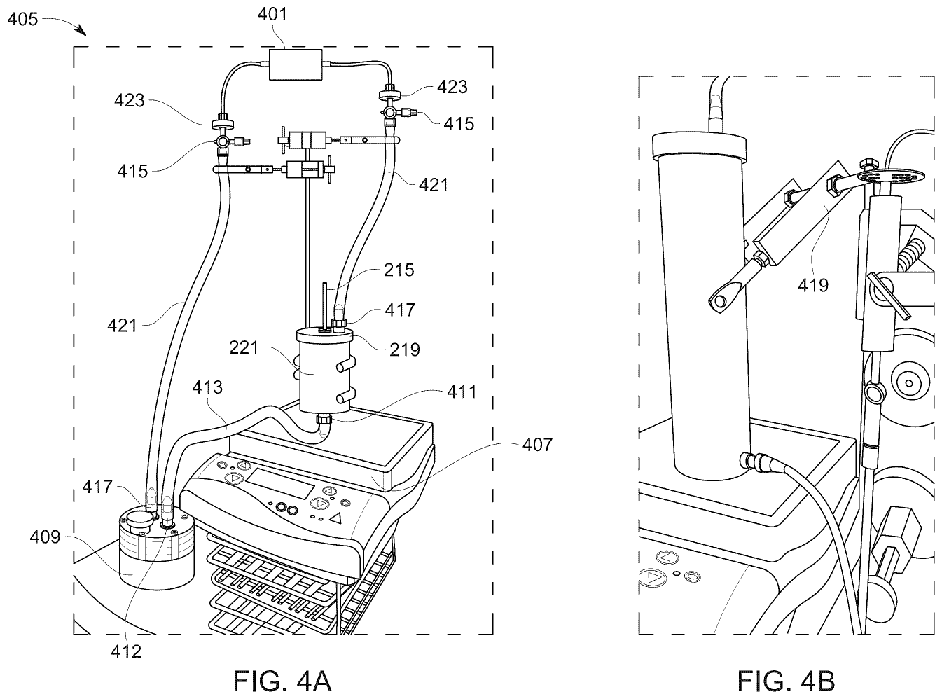

In certain embodiments, the population of ASCs is subjected to a perfusion flow induced by a bioreactor system 405 (FIG. 4A) which provides a dynamic culture environment closer to the physiological conditions, to produce a population of differentiated cells and/or tenocyte-like cells. Culturing the cells in the bioreactor system 405 for a period of time (e.g., ranging from 3 to 180 days, preferably from 7 to 42 days) increases cell proliferation and tenogenic commitment of ASCs. ASCs assume a tendon progenitor cell-like morphology, express tendon-related genes, and produce more organized extracellular matrix in tenogenic versus stromal medium with perfusion and centrifugal fluid motion. In another aspect of the embodiments, the perfusion flow increases cell viability by providing a more homogeneous distribution of nutrients and mass products (e.g., toxic metabolites) within the culture system. In yet another aspect of the embodiments, the flow perfusion induces shear stresses as mechanical cues to drive tenogenic commitment, which would only be obtained with tenogenic soluble factors in a traditional static culture system. The differentiated or mature cells maintain high capacity for self-renewal and cell proliferation, uniform distribution and viability within the collagen sponge seeded or infused with ASCs (describe elsewhere in more detail) under controlled biochemical and mechanical stimulations. The circulation of the flow of medium in the bioreactor facilitates the uniform cellular distribution and cellular viability within the ASC-scaffold construct.

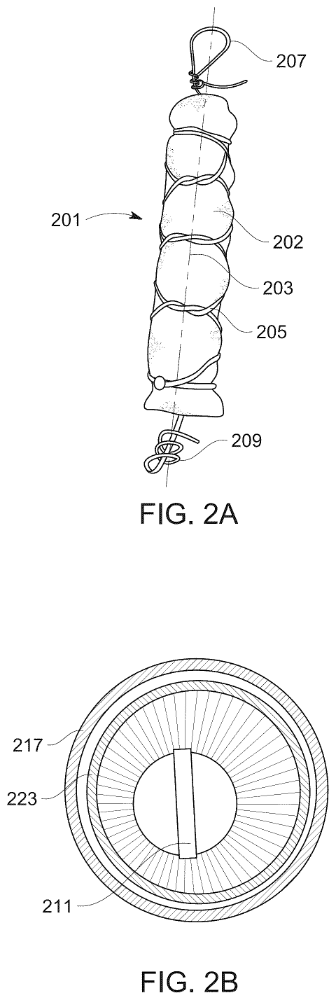

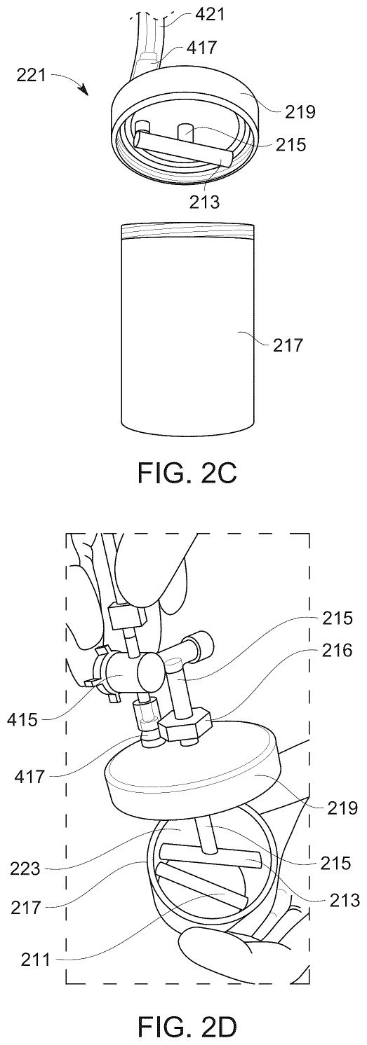

In tendon, the deposit of ECM components and secretion of soluble factors maintain the balance of tissue homeostasis such as cell differentiation. In certain embodiments, the tenogenic differentiation drivers include scaffold biopolymers which can mimic ECM components as well as soluble tenogenic growth factors, particularly transforming growth factor beta (TGF-β). Examples of TGF-β affecting the tenogenic differentiation of stem cells include but not limited to TGF-β1, TGF-β3 or bone morphogenetic protein 12 (BMP12). In one aspect of the embodiments, the differentiated cells may be cultured in a medium supplemented with an additional tenogenic driver to further maintain the teno-lineage or mature the differentiated cells into tenocyte-like cells. The scaffold 201 illustrated in FIG. 2A may comprise biomimetic molecules of biological and/or synthetic origin suitable for tendon tissue engineering. In one aspect of the embodiment, the biopolymer-based scaffold 201 includes collagen of type I at a concentration of between 60 to 80% and is also referred to herein as a COLI scaffold. Collagen is the most abundant natural polymer in the body and is biocompatibility. Several collagen-based scaffold formulations are currently commercially available and FDA-approved. (Snedeker, J. G. & Foolen, J. Tendon injury and repair-A perspective on the basic mechanisms of tendon disease and future clinical therapy. Acta biomaterialia 63, 18-36, doi: 10.1016/j.actbio.2017.08.032 (2017); Cockerham, K. & Hsu, V. J. Collagen-based dermal fillers: past, present, future. Facial plastic surgery: FPS 25, 106-113, doi: 10.1055/s-0029-1220650 (2009)). The biopolymer-based scaffold 201 may further include other components of ECM, including collagen of type II, preferably cross-linked. Non-limiting examples of molecules that may be added to the scaffold 201 are molecules naturally occurring in tendon such as tenomodulin and fibromodulin, and/or other molecules including laminin, fibronectin, PLA, PGLA, PLLA, PEEK, PEG, and elastin. In one aspect of the embodiments, the molecule may act as tenogenic differentiation driver to enhance the tenogenic differentiation and/or the maturation process.

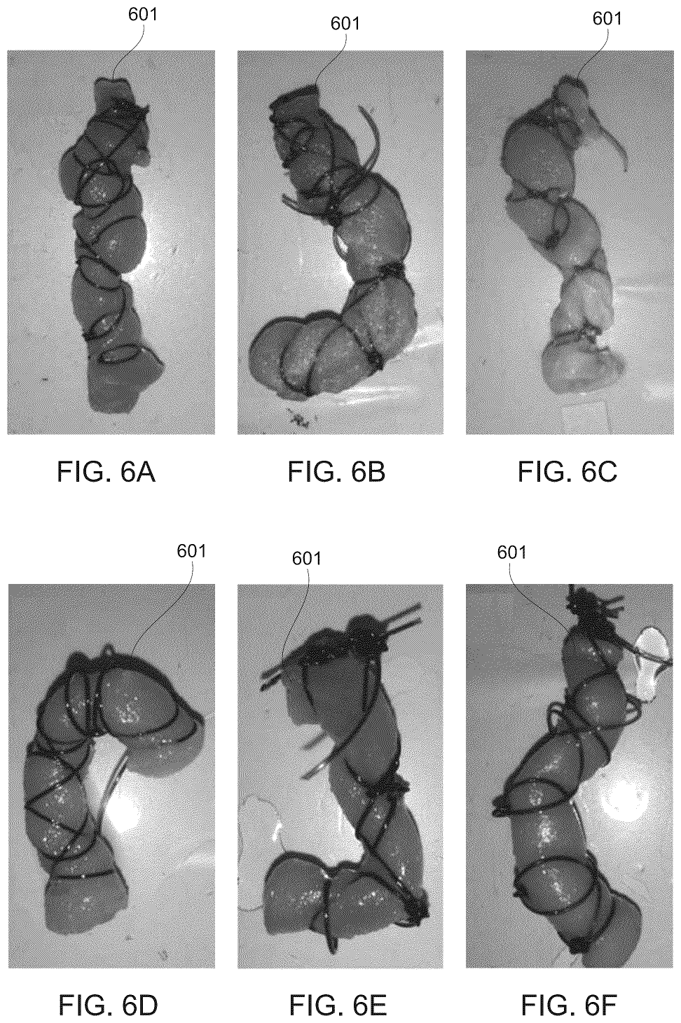

A preferred example of collagen-based scaffold 201 may be a collagen sponge such as COLI. (FIGS. 2A and 6) With its biological origin, such collagen sponge can provide a microenvironment rich in collagen proteins creating stem cell niches where ASCs can attach, proliferate, and differentiate. In one aspect of the embodiment, the collagen sponge may be reinforced with synthetic biopolymers. In another aspect of the invention, the collagen sponge is not reinforced with synthetic biopolymers and may be subjected to controlled mechanical tensions and stress allowing the migration of cells into the scaffold 201 for a uniform cell distribution, and adequate exchange of oxygen and nutrients between the cell-scaffold construct and the medium.

Another example of scaffold 201 may be cell-derived decellularized matrices which comprise bioactive, biocompatible materials made up of fibrillar proteins and other factors that recapitulates features of the native structure and composition of tendon.

Another example of scaffold 201 may be a hydrogel where the seeded cells are embedded within during solidification, allowing viable ASCs to adhere to collagenous fibrils.

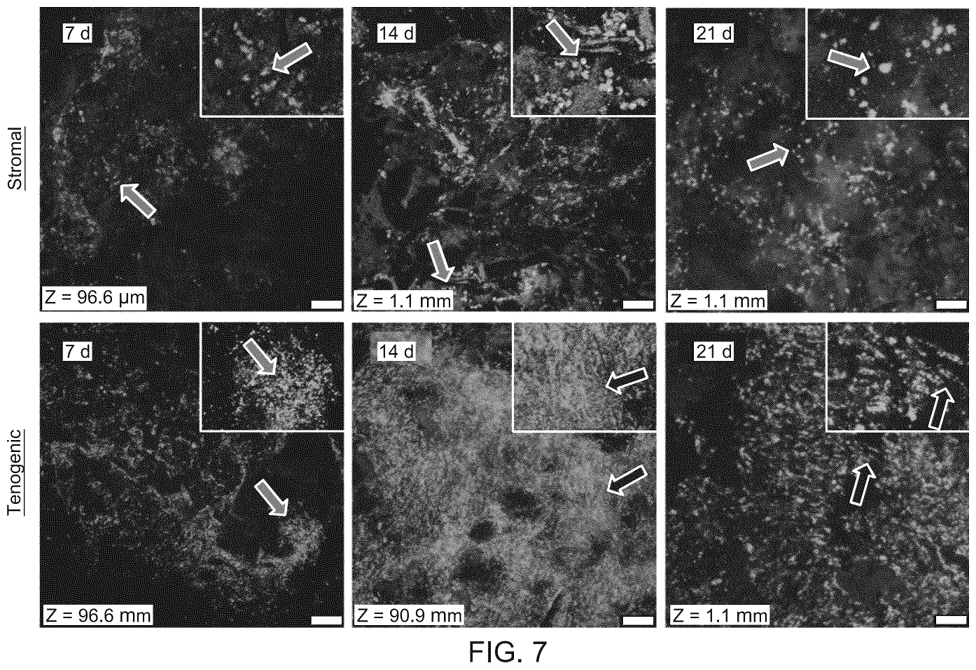

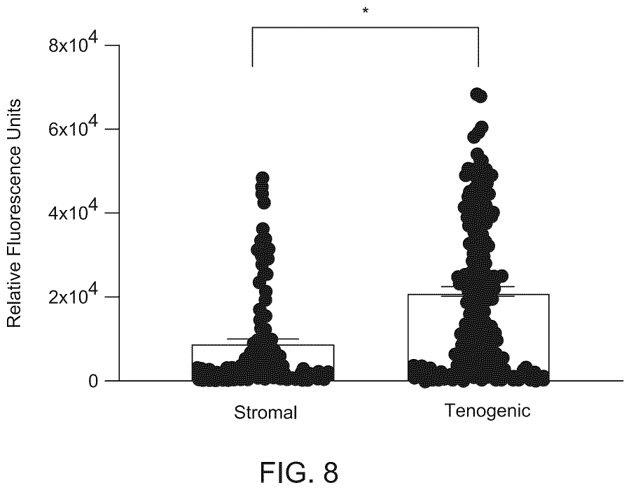

As opposed to hydrogel embedding of ASCs, culturing of ASCs onto collagen sponge has limited success. Cell distribution in COLI sponge is more challenging than in the stable architecture of decellularized tissue or malleable, semi-solid hydrogels with low fiber density and stiffness. The reason for low cellular adherence and differential cellular distribution may be duc to pore closure upon hydration of the sponge, which prevents migration of cells onto the scaffold 201 and exchange of oxygen and nutrients. Culture mechanisms with dynamic fluid flow according to the present invention are designed to mitigate these limitations. Shear stresses and better gas and nutrient delivery from centrifugal fluid motion, for example generated with a stir bar, may also improve ASCs proliferation on collagen sponges. In addition, reinforcement of mechanical properties of sponges with synthetic polymers has been demonstrated to be effective. In certain embodiments, the methods unexpectedly achieve cellular proliferation in tenogenic medium without reinforcement of the scaffold with synthetic biopolymers. The cells show high proliferative capacities within the cell-scaffold construct (also referred to herein as the cell construct or an ASC-COLI construct) 601 which may stem from high shear stress produced by higher stirring rate in methods of certain embodiments. In general, higher stirring rate leads to increased degradation of the collagen sponge. In some instances, collagen sponges may lose as much as 50% of size and surface after 7 days of stirring culture at 150 rpm without synthetic polymer reinforcement. Similarly, ASC-scaffold constructs 601 of some embodiments show loss of collagen when cultured in stromal medium lacking a soluble tenogenic differentiation driver such as TGF-β1. (FIGS. 7 and 8)

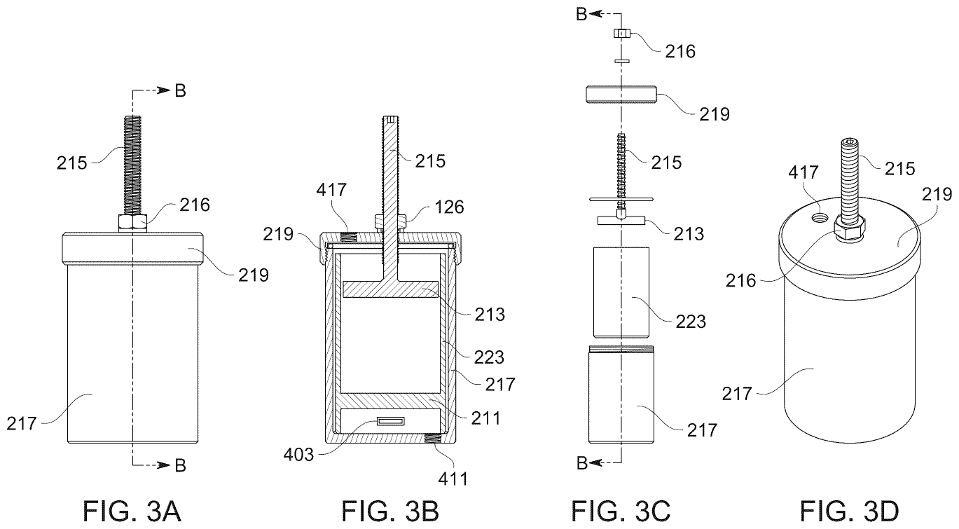

As illustrated in FIG. 2, in certain embodiments, the scaffold 201 is shaped as a column along a longitudinal axis 203, for example by being ligated by a filament, suture or net 205. In one aspect of the embodiments, the scaffold 201 is rolled into a column wrapped by a finger trap composed of polydioxanone suture 205 with loose loops 207, 209 on each end to secure the construct to an immobile horizontal bar 211 formed on a core frame 223 within a base chamber 217 and one adjustable horizontal bar 213 attached to a threaded vertical bar 215, the threaded vertical bar being locked in the top lid 219 via a nut 216 (FIGS. 2A-2D, 3A-3D) In onc embodiment, ASCs may be directly applied to the scaffold 201 or infused onto the scaffold 201 to form the cell-scaffold construct 601. After culturing over a period of time, the cell-scaffold construct 601 may contract in areas separated from the filament or net 205. In one aspect of the embodiment, the extremities of the cell-scaffold construct 601 display significant contractions compared the middle area of the cell-scaffold construct, in particular at the end of the culture period. (FIGS. 6D-6F)

In certain embodiments, tendon neotissue cultured in tenogenic medium displays gross stiffness overtime while not showing gross appearance of degradation despite stirring of medium at relatively high speed of between 10 to 10,000 rpm, preferably 300 rpm) for a prolonged culture period (e.g., 21 days). The collagen-based scaffold 201 may be naturally reinforced with abundant ECM progressively deposited by tenogenically induced ASCs.

Collagen-based scaffold 201 may be used for delivery and retention of stem cells in tendon and ligament tissue, and support differentiation of ASCs into cells of the tenolineage. As such, they can be implanted in vivo on sites where the tendon or ligament injuries have occurred.

In certain embodiments, the population of ASCs is infused or seeded onto a porous biopolymer-based scaffold 201 to form a cell-scaffold construct 601. In one aspect of the embodiments, the perfusion flow facilitates ASC seeding or attachment onto the biopolymer-based scaffold 201 to form the cell-scaffold construct 601. In other embodiments, the population of ASCs is directly applied onto the biopolymer-based scaffold 201 to form the cell-scaffold construct 601. For example, the population of ASCs is seeded onto the scaffold at a density of between 5.5×10⁰ and 1.0×10⁹ ASCs/cm³, preferably 1.0×10⁶ ASCs/cm³.

In certain embodiments, the cell-scaffold construct 601 is cultured in the bioreactor system 405 and subjected to biochemical stimulations and controlled mechanical loadings for a period of time (from 3 to 180 days, preferably 7 to 42 days) to produce a population differentiated cells and/or tenocyte-like cells. In one aspect of the embodiments, the cells are cultured in a medium supplemented by at least one tenogenic differentiation driver which provides biochemical cues for differentiation into the tenolineage. For example, the biochemical stimulation may be a soluble factor of the TGF family, preferably TGF-β1. In other embodiments, differentiated cells do not exhibit a homogeneous phenotype but rather form a mixed culture of cells being at different stages of the tenogenic differentiation process and as such having different phenotypes.

In an embodiment, the benefits of the dynamic perfusion are like those of centrifugal forces, but the shear forces are aligned with the longitudinal axis 203 of the construct, while the centrifugal fluid flow generates tangential shear forces. Centrifugal and perfusion fluid flow are uniquely combined in the bioreactor 221. Notably, the magnitude of dynamic perfusion and centrifugal fluid motion may be customized for bioreactor and construct configuration and size.

In one aspect of the embodiment, the controlled mechanical stimulations include flow shear stress and dynamic or static tensile strain. The flow shear stress is induced by the flow of medium circulating throughout the bioreactor system 405: (i) the perfusion flow and (ii) the centrifugal flow motion of the medium. As illustrated in FIG. 4A, the perfusion flow is generated via a pump 401 which controls the rate and directionality of the flow. In an embodiment, the pump 401 is a peristaltic pump and generates a bidirectional flow with a rate of between 2 and 50 ml/min, preferably about 10 ml/minute in the bioreactor 405. In addition, an agitator, for example a magnetic stir bar 403, creates a centrifugal flow motion which not only induces flow shear stress on the cells, but also enhances convective mass transport. For example, the centrifugal flow motion may have a speed of between 10 and 10,000 rpm, preferably 300 rpm. As illustrated in FIG. 4B, a cell-scaffold construct secured within the core frame 223 is cultured under dynamic strain, for example using a cyclic tensioner 419.

In one aspect of the embodiments, the cell-scaffold construct 601 is further subjected to a dynamic or static tensile strain. Applying a strain onto the cell-scaffold construct 601 over a period of time mimics the mechanical loads transmitted through the ECM in vivo to tendon cells while also maintaining cell viability in culture. The dynamic or static tensile strain affects tendon gene expression, cell morphology, protein production as well as gross tissue properties including cellular alignment and tensile strength. In one aspect of the embodiment, the tensile strain is static and uniaxial, generated by elongating the cell-scaffold construct along its longitudinal axis. (FIG. 4A) Uniaxial tensile strain more closely replicates loads experienced by native tendon cells. For example, the static tensile strain may have an amplitude of between 1 to 75%, preferably 10%. In another aspect of the embodiments, the duration of the tensile strain may influence the tenogenic differentiation and cell morphology. For example, the tensile strain is preferably static and applied over the prolonged culture period. In another aspect of the embodiments, the cell-scaffold construct 601 is exposed to a tensile strain for short periods of time over the prolonged culture period to prevent the cells from developing stress tolerance for mechanical loadings. In another aspect of the embodiments, the strain is not static but dynamic and may have an amplitude from about 1 to 75%, preferably about 10%. (FIG. 4B) In yet another aspect of the embodiments, the tensile strain may alternatively be applied as static strain and dynamic strain.

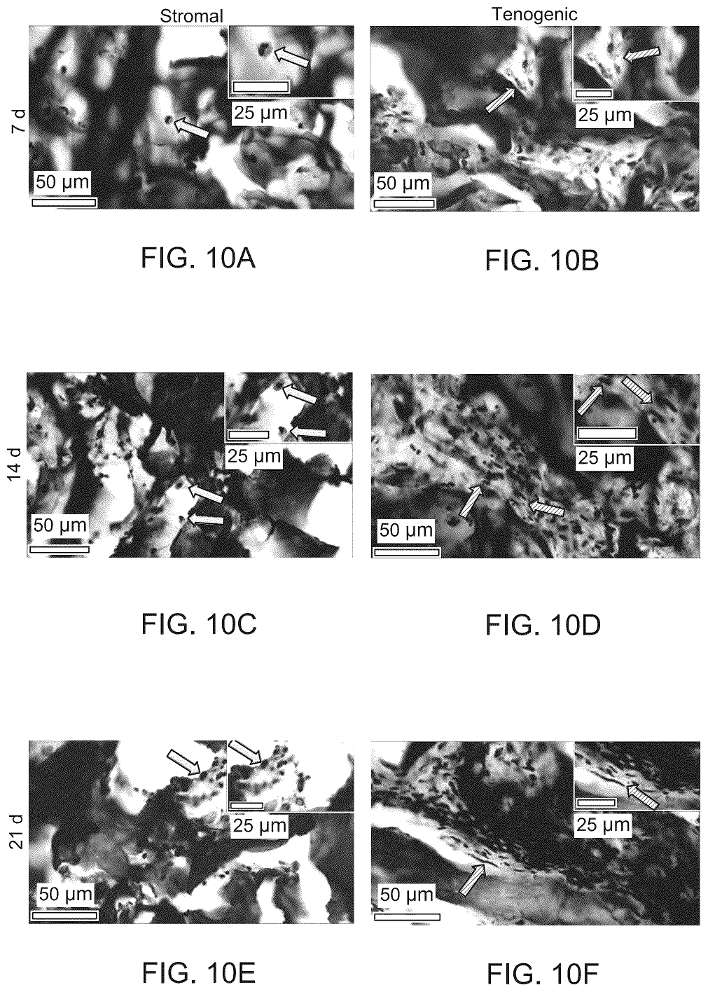

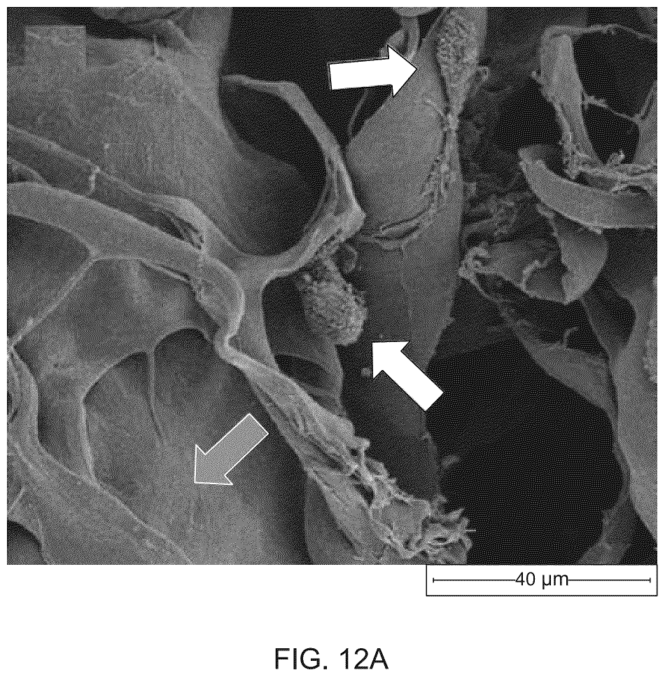

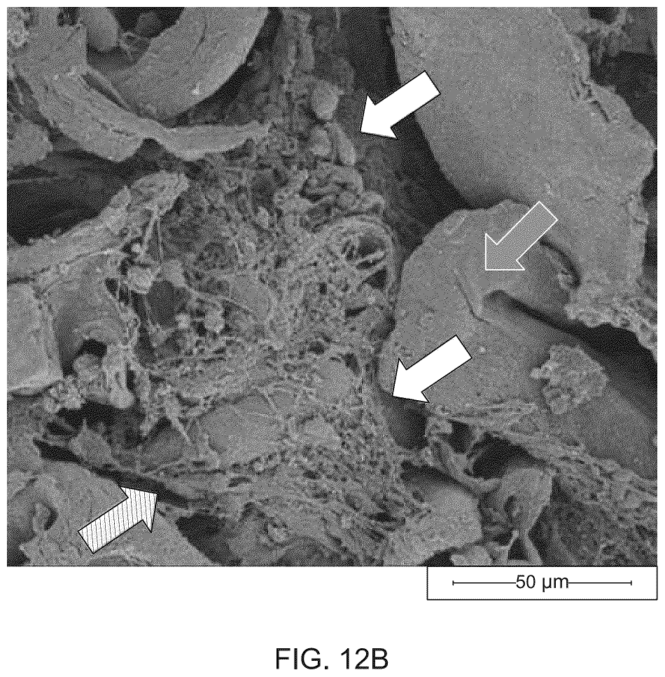

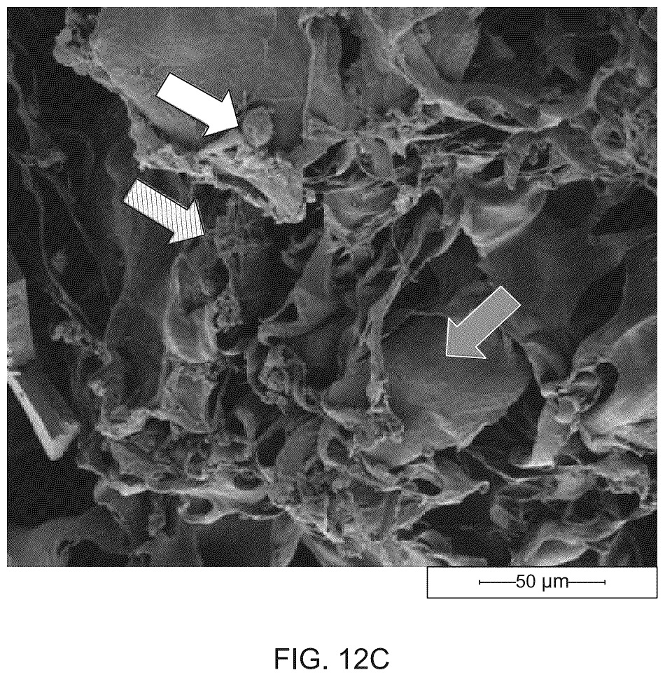

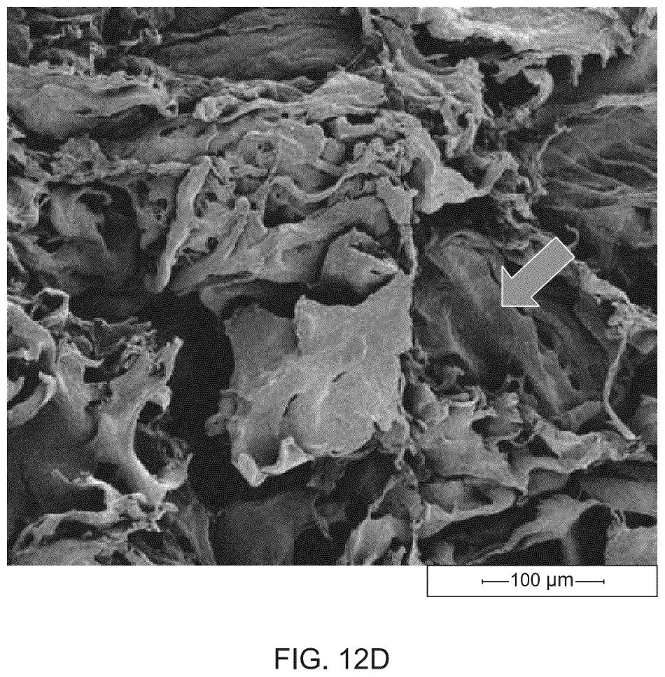

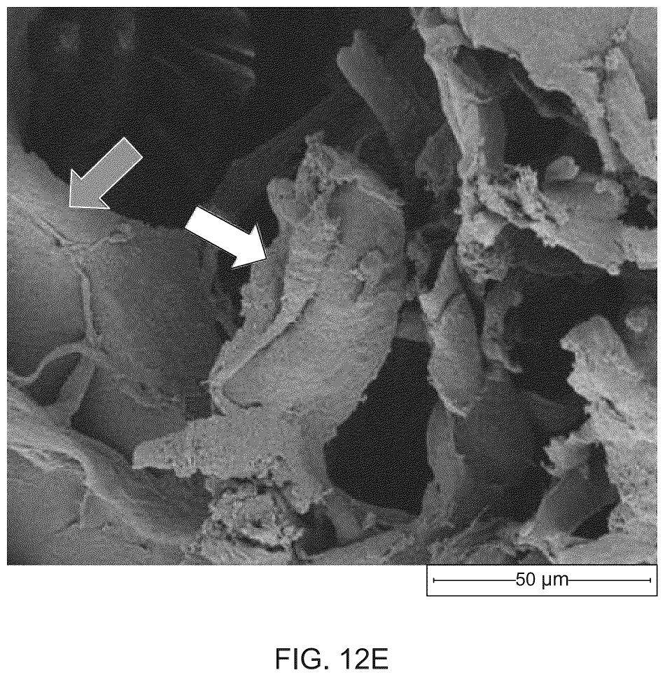

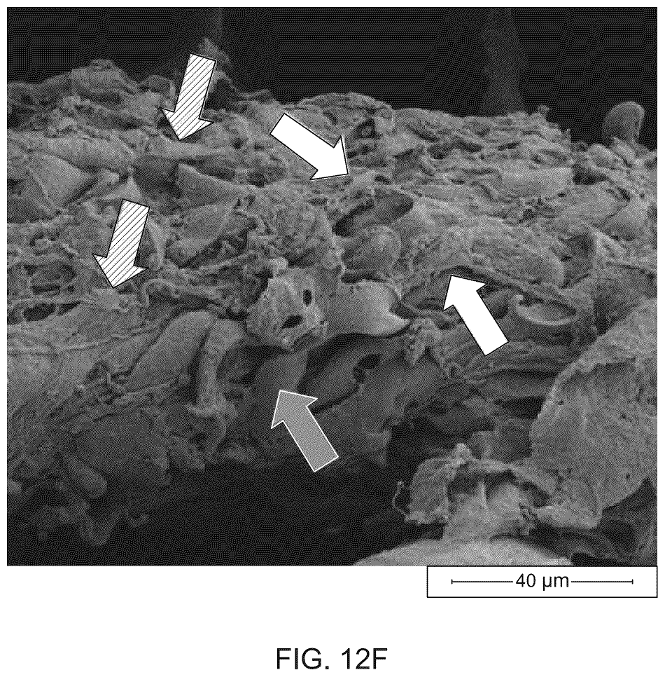

In certain embodiments, culturing a population of ASCs under static or dynamic tensile strain produces many differentiated cells which are organized parallel to each other on the collagen fibrils of the scaffold. The cell-scaffold constructs subjected to dynamic or static tensile strain and cultured in tenogenic medium displays a much higher and more densely populated scaffold than the constructs not subjected to tensioning and cultured in the same medium. The cells show high capacity for proliferation, especially when cultured in the tenogenic medium (FIGS. 7-8) Higher shear stress of fluid flow in swirling motion creates an optimum mechanical stimulus to orient the cells in parallel alignment in tenogenic medium without the use of dynamic tensioning apparatus. Further, from TEM images, the collagen fibrils deposited around the tenocyte-like cells observed only in construct cultured in tenogenic medium was of characteristic to tenocytes and demonstrated differentiation and maturation of the cells. (FIGS. 10 and 12)

In certain embodiments, construct strain is imperative to effective tenogenic differentiation of progenitor cells, and variations in dynamic or static strain affect cell proliferation and the rate and efficiency of tendon neotissue generation. During culture, cells align with the direction of construct strain, for example in the direction of the longitudinal axis of the construct 203. In one aspect of the embodiment, the maintenance of a construct strain of 10% promotes cell distribution and alignment along tensioned COLI fibers and support cell proliferation and differentiation. In another aspect of the embodiment, the culture conditions alone are not sufficient to drive cell differentiation and ECM deposition. Accordingly, medium may be supplemented with components, such as TGF-β which is a critical growth factor, capable of inducing differentiation of progenitor cells into tenocytes. Differentiation is regulated by TGF-β/Smad2/3 signal transduction, and TGF-β also promotes cellular production of collagen.

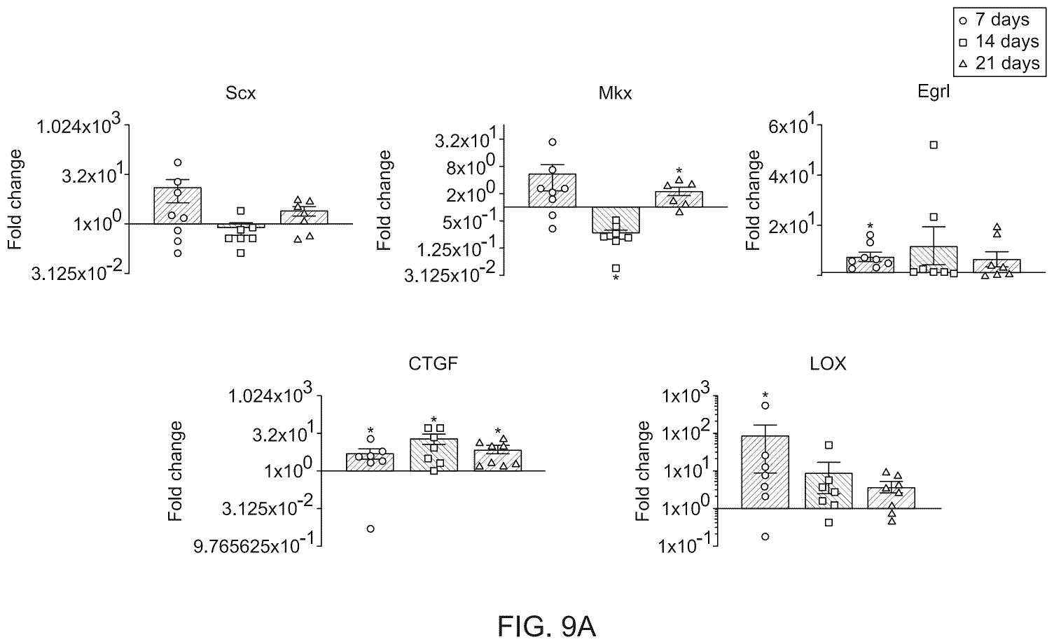

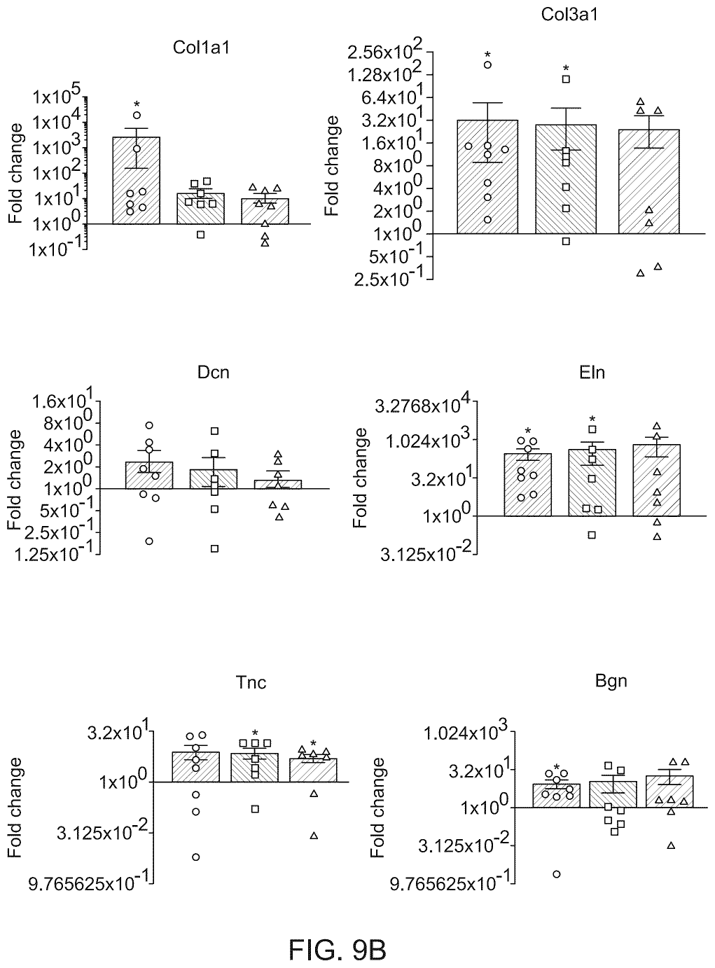

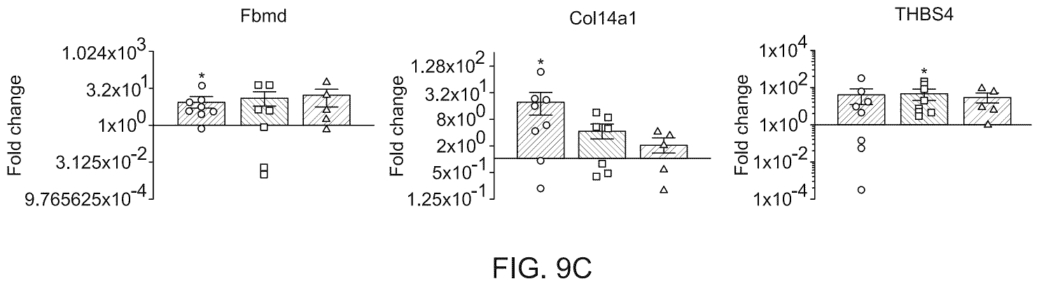

In certain embodiments, the ASCs-scaffold construct 601 cultured in the bioreactor system 405 under at least one biochemical stimulation and controlled mechanical loadings produce a population of differentiated cells, expressing at least one tenogenic transcription factor, at least one tenogenic extracellular matrix gene, or a combination thereof. Tenogenic transcription factors may be upregulated early during the differentiation process of the population of ASCs when cultured in tenogenic medium under biomechanical stimulations. In some embodiments, early upregulation of certain genes occurs and is subsequently followed by gradual return to baseline. Early upregulation and activation of transcription factors may lead to an increase of expression of genes responsible for induction and progression of specific-cell type differentiation. Examples of transcription factors include but are not limited to scleraxis (Scx), mohawk (Mkx), early growth response 1 (Egr1), connective tissue growth factor (CTGF) and lysyl oxidase (LOX). (FIG. 9A) For example, the transcription factor Scx is overexpressed at the earliest stage during the tenogenic differentiation process (day 7) and can induce the tenogenic differentiation and directs ASCs into tenogenic lineage. Sex may prevent ASCs to differentiate into chondrogenic cells under the influence of a biochemical stimulation such as TGF-β1. The expression of Mkx is also upregulated in tenogenic medium compared to stromal medium over the prolonged culture period, thus indicating that cells maintained active differentiation to tenocytes throughout the culture period. (FIG. 9A) Mkx is another important tenogenic transcription factor, which has been shown to prevent progenitor cells to differentiate into myofibroblasts at the onset of the healing process, hence impedes angiofibrosis. In some embodiments, the expression of Egr1 is upregulated in the early period of the differentiation process e.g., at day 7. The expression pattern corroborates with that of healing tendon in rabbit model, demonstrating the highest expression of Egr1 at the injury site after 7 days post-injury, followed by a return to original levels by day 28.

In one aspect of the embodiments, CTGF gene expression is upregulated in tenogenic medium compared to stromal medium throughout the prolonged culture period, without clear distinction between early and late culture periods. (FIG. 9A) CTGF is among the highest expressed constitutive tenogenic transcription factors and may be essential for tenogenic differentiation, especially in inducing ECM production. CTGF has been shown to prevent aging of tenogenic progenitors. In the tendon repair process, CTGF had been upregulated only at day 3 post-injury in chicken compared to the baseline. The early upregulation of CTGF in the method of the embodiments suggests that the culture conditions (i.e., 3D culture, biochemical and mechanical stimulations) recapitulate the embryonic development of tendon tissue more closely than the repair process.

In certain embodiments, LOX is overexpressed early in the differentiation process followed by a gradual decline, although the expression levels are higher in tenogenic medium compared to stromal medium at early stage and late stage of the culture period (e.g., 7 and 21 days of culture). (FIG. 9A) ASCs upregulate LOX in transected calcaneal tendon of rat at day 14 compared to non-treated or ASCs combined with growth differentiation factor (GDF)-LOX is an enzyme required for cross-linking of collagen fibrils in tendons and plays an essential role in translating embryonic movements into development of mechanical properties during gestation. Inhibition of LOX during embryonic development severely impairs collagen crosslinking and mechanical properties of tendon. The high expression of LOX at early stage of the culture followed by a decline suggests that tendon neotissue produced according to the methods mimics embryonic tendon development.

The population of differentiated cells produced according to the methods of certain embodiments express at least one tenogenic extracellular matrix gene. Examples of tenogenic extracellular matrix gene include but are not limited to collagen 1a1 (Col1a1), collagen 3a1 (Col3a1), decorin (Dcn), elastin (Eln), tenascin-c (TnC), and biglycan (Bgn). (FIG. 9B)

In one aspect of the embodiments, Col1a1 gene expression pattern followed a similar trend than the transcription factors. (FIG. 9B) Early upregulation of Col1a1 (e.g., at day 7) may be similar to the early healing response to tendon injury. In equine tendons, Col1a2, another subunit of collagen type I, expressed constitutively in both adult healthy tendons and acutely injured tendons, whereas expression was lower in tendons with chronic tendinopathy. In one aspect of the embodiment, gene expression of Col3a1 was upregulated and relatively stable throughout the culture period as opposed to Col1a1. Both genes are essential in maintaining mechanical properties of tendons. The expression of both genes can be abolished by lack of mechanical stimulus, as demonstrated in a study with dogs whose digital flexor tendon was unloaded by limb suspension and all collagen type I, II, and III expressions were severely impaired by day 42. However, Col1a1 and Col3a1 were shown to upregulate in mice digital flexor tendon upon transection up to day 21 by gradual increase, yet both expressions were negligible by day 28. (FIG. 9B)

In one aspect of the embodiments, TnC gene expression is upregulated. TnC is normally expressed at negligible level in tendon, while being upregulated upon injury. (FIG. 9B) Indeed, TnC is most likely an essential gene for tendon development and healing process in horses. By comparing the expression levels of TnC among equine fetal, yearling, and adult tendons, the expression increases from fetal to yearling and returns to fetal level in adults. Similarly, its expression is high in acutely injured tendons, while it is low in healthy tendons or those affected with chronic tendinopathy.

In the context of the invention, gene expression results are relative to the comparisons between culture conditions. Taken together, the transcription factor, extra-cellular matrix, and mature tendon gene expression profiles suggest tenogenic differentiation of ASCs on COLI constructs cultured in tenogenic medium relative to those cultured in stromal medium.

In certain embodiments, the population of differentiated cells expressing at least one tenogenic transcription factor and/or at least one ECM gene comprises a mixed population of cells at various differentiation stage. In one aspect of the embodiments, the differentiated cells undergo progressive morphological changes, becoming elongated and assuming a spindle shape. The cells tend to form clusters which grow larger as the number of cells increases. Notably, the cells are embedded in newly deposited fibrous ECM network. In one aspect of the embodiments, the deposit of ECM components produced by the cells themselves progressively fill the pores of the scaffold consolidating the cell-scaffold construct, thereby providing further support and stimulations for the differentiation process. The differentiated cells align parallel to each other along the cell-scaffold construct 601 longitudinal axis 203.

The population of differentiated cells produced according to the methods of certain embodiments is further cultured under the same culture conditions (i.e., 3D culture, biochemical cues and mechanical loadings) to produce mature cells having a tenocyte-like phenotype. In certain embodiments, the tenocyte-like phenotype is defined by the expression of at least one tendon marker gene. Examples of tendon marker gene include but are not limited to fibromodulin (Fbmd), collagen 14a1 (Col14a1), tenomodulin (Tnmd), and truncated hemoglobin 4 (THBS4). (FIG. 9C)

In one aspect of the embodiments, the expression of the tendon marker gene is initially upregulated with a sustained expression towards the end of the culture period.

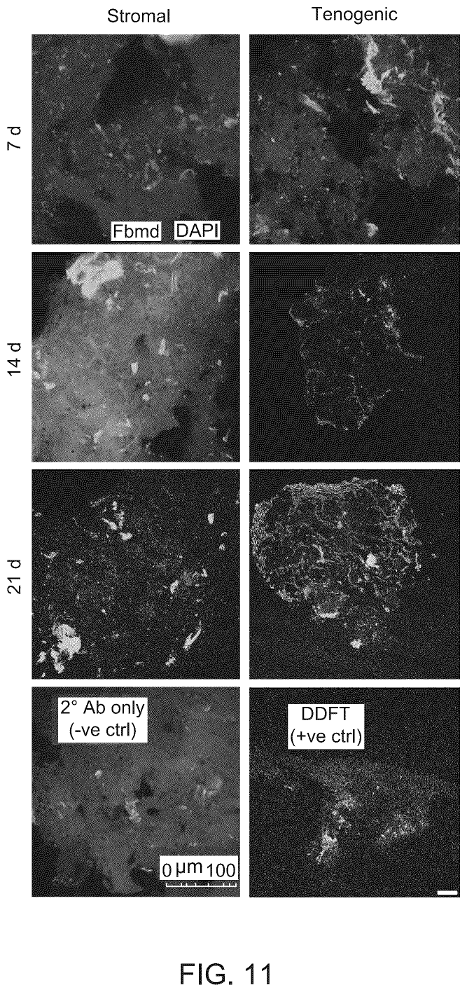

As a maturation marker, Fbmd is highly and constitutively expressed in tendons. Its expression transiently decreases upon injury while gradually increasing over the healing period. Fbmd shows monotonic increase in expression throughout the gestation and up to 14-day after birth in mice patellar tendon suggesting its crucial role during the embryonic development of tendons. In one aspect of the embodiments, there was no deposition of Fbmd onto the cell-scaffold construct in the early stage of the differentiation process. (FIG. 11, right top panel) The Fbmd expression and deposition progressively increase throughout the differentiation process, resulting in extensive amounts of Fbmd deposited throughout the constructs by the end of the culture period (e.g., day 21). (FIG. 11, right second bottom panel) In an embodiment, fibrous structure of Fbmd is deposited parallel to collagen fibers of the construct, thereby suggesting that the differentiated and/or mature cells deposited Fbmd directly onto collagen fibers of scaffold. (FIG. 11, right second bottom panel)

In another aspect of the embodiments, the differentiated cells which undergo maturation express Col14a1 followed by gradual return to basal expression. (FIG. 9C) Col14a1 is an early regulatory marker for neotendon maturation. In fact, normal development of mice flexor digital tendon shows strong expression of Col14a1 both at the gene and protein levels until 10 days after birth, but the expression diminishes by 30 days after birth. Knockout of Col14a1 negatively affects mechanical strength of mice tendon at early stage but not late stage. Additionally, a tenogenic construct made of hydrogel and tenocytes showed higher expression of Col14a1 at day 7 of culture and later downregulated by day 45.

In another aspect of the embodiments, THBS4 is highly upregulated throughout the duration of tenogenic differentiation process, although statistical significance compared to stromal medium was achieved only at day 7 of culture in tenogenic medium. (FIG. 9C) THBS4 has been reported as a tendon ECM composition modifying protein, and knockout mice demonstrated compromised limb strength due to weakened muscle. In ovine calcancal tendon, THBS4 is also highly expressed in early gestation and gradually decreases to low expression at adult tendon. In the embodiment, relatively high expression of THBS4 suggests that the methods of the embodiments more closely resemble the developing tendon tissues.

In one aspect of the embodiments, tenomodulin (Tnmd) is expressed in large amount at the end the differentiation process after a gradual increase throughout (e.g., 21 days of culture in tenogenic medium). In contrast, the gene expression in DDFT tissue was relatively low. Progressive increase of tenomodulin staining in tenogenic medium-cultured construct during the culture period indicates maturation of tenoblast-like cells at early culture earlier stages to tenocyte-like cells at later culture stages. Tenomodulin is a transmembrane glycoprotein that is highly expressed in tendons, ligaments, and other poorly vascularized tissues. Tenomodulin was found to be necessary for both tenocyte proliferation and maturation. As a transmembrane protein of cells, nearly all area of a tendon are usually positively stained with anti-tenomodulin antibody. Tenomodulin is generally considered maturation marker, yet the expression level does not increase monotonically with age, and the trend varies depending on the area of tendon. For example, its expression monotonically increases and peaks during gestation and gradually decreases by 14 days postnatal in mouse patellar tendon. Moreover, the reduction is more acute at the tibial insertion than mid-substance. To date, there has been no report on tenomodulin expression in equine DDFT. The findings of this study are consistent with the expression pattern of mouse patellar tendon, as highly proliferative neotendon at 21 days of culture in tenogenic medium had much more abundant presence of tenomodulin than healthy DDFT from mature horse. The importance of tenomodulin expression in tendon healing was also evident from a study that found low expression of tenomodulin in tetranectin-null mice that had delayed healing, indicating the essential role tenomodulin plays during late phase of embryonic development and healing from injury. Based on the tenomodulin expression pattern, the differentiation and maturation process most likely resemble developing tendon before fully maturing.

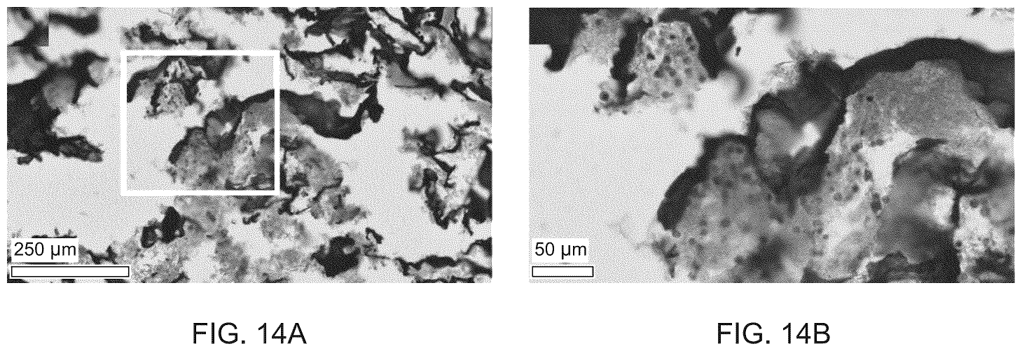

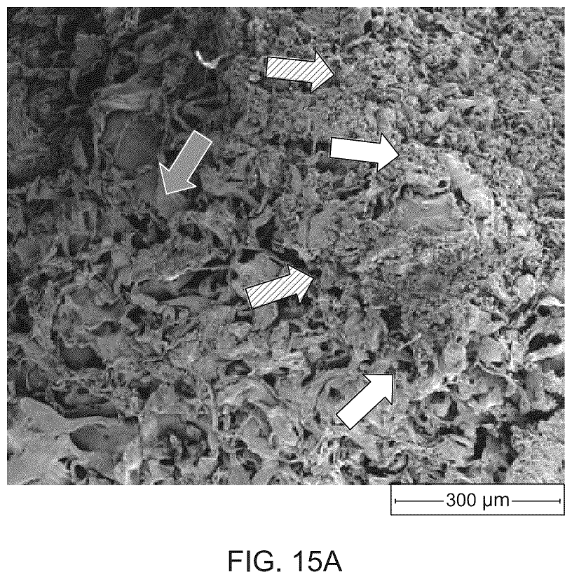

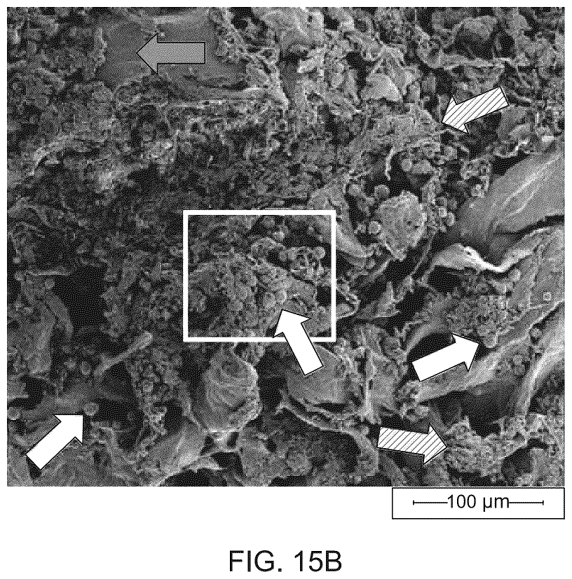

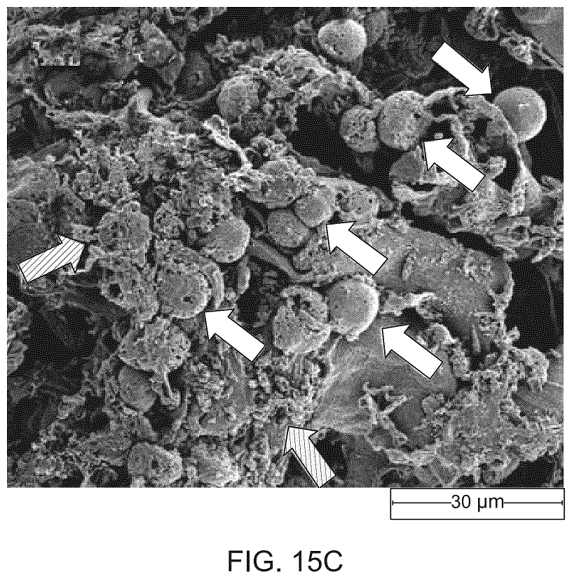

In certain embodiments, the tendon neotissues are produced from populations of ASCs which display the desirable characteristics of native tendon tissue including a well-aligned cellular morphology, the differentiation of the ASCs towards a tenocyte-like phenotype, and the production of ECM proteins commonly observed among tendon tissues. The ASCs undergo a differentiation process when cultured with at least one tenocyte differentiation driver, flow shear stress and static tensile strain. (FIGS. 10B, D, F and 12D, E, F) Accordingly, the methods of producing a tendon neotissue from a population of ASCs cultured in a bioreactor system, includes: infusing or directly applying the population of ASCs onto a porous biopolymer-based scaffold to form a cell-scaffold construct, culturing the cell-scaffold construct in a medium for a period of time to produce a population of tenocyte-like cells, comprising: contacting the cell-scaffold construct with at least one tenogenic differentiation driver, and applying controlled mechanical stimulations to the cell-scaffold construct comprising: flow shear stress, and static tensile strain, wherein said population of tenocyte-like cells express at least one tendon marker gene and organize to form said tendon neotissue. In other embodiments, the ASCs undergo a differentiation process when cultured with at least one tenocyte differentiation driver, flow shear stress and dynamic strain. (FIGS. 14A-14B, FIGS. 15A-C). Accordingly, the methods of producing a tendon neotissue from a population of ASCs cultured in a bioreactor system, includes: infusing or directly applying the population of ASCs onto a porous biopolymer-based scaffold to form a cell-scaffold construct, culturing the cell-scaffold construct in a medium for a period of time to produce a population of tenocyte-like cells, comprising: contacting the cell-scaffold construct with at least one tenogenic differentiation driver, and applying controlled mechanical stimulations to the cell-scaffold construct comprising: flow shear stress, and dynamic strain, wherein said population of tenocyte-like cells express at least one tendon marker gene and organize to form said tendon neotissue.

In certain embodiments, the tendon neotissue comprises the population of tenocyte-like cells are embedded within a fibrous extracellular matrix (ECM) attached to the biopolymer-based scaffold (e.g., collagen) and are organized parallel to each other along the biopolymer-based scaffold longitudinal axis. In one aspect of the embodiments, the tendon neotissue may further comprises other immature differentiated cells and/or tenoblast-like cells. The tendon neotissue may comprise an abundant ECM made of elongated collagenous fibers and elastic fibers embedding tenoblast-like cells and tenocyte-like cells. In one aspect of the embodiments, the tendon neotissue mimics the physiology and structural organization of native tendon with low cellularity, and high content of ECM and collagenous fibers. For example, the collagenous fibers have a high content of collagen of type I, preferably between 60 and 80%. The ECM materials surrounding the collagenous fibers and elastic fibers may comprises additional molecules such as glycosaminoglycans, proteoglycans and other small molecules. Changes in cell and ECM morphology parallel gene expression and are consistent with tenogenic differentiation. A different appearance, greater adhesion between construct layers, and firmer texture of constructs cultured in tenogenic medium are consistent with tenogenic differentiation and aligned with the more highly organized, fibrillar ECM visible at the micro- and ultra-structural levels. Specifically, cells in tenogenic medium bad the appearance of differentiated cells surrounded by collagen-like fibrils, while those in stromal medium had the appearance of immature progenitor cells. Immunolocalization of fibromodulin, a bioactive factor of native tendon ECM suggested to be a differentiation marker of tendon, additionally supports the presence of tendon progenitor cells in the constructs cultured in tenogenic medium. The protein is a vital component of the tendon progenitor cell ECM niche that controls tendon progenitor cell self-renewal and differentiation and also regulate collagen fibril size by controlling premature cross-linking via LOX modulation. The pattern of fibromodulin labeling in the constructs cultured in tenogenic medium resembled that of mature equine where the strongest staining is in the intrafasicular matrix surrounding bundles of collagen called fascicles. Lower, poorly organized labeling in constructs cultured in stromal medium highlights some low level of cell differentiation. The gross appearance, micro- and ultrastructure, and immunohistochemical labeling of constructs cultured in tenogenic medium are consistent with early tendon neotissue.

### Implant

The unique combination of centrifugal and perfusion fluid flow of tenogenic medium supports de novo tendon neotissue from adult equine ASCs. De novo equine tendon neotissue tissue may be a useful resource for investigations surrounding tendon formation and pathology. This is especially important to reduce animal use for screening therapeutic compounds and technology.

In certain embodiments, an implant comprises at least one tendon neotissue produced after tenogenic differentiation of a population of ASCs into a population of differentiated cells expressing at least one tenogenic transcription factor, at least one tendon-specific ECM gene or combination thereof. In one aspect of the embodiments, the population of differentiated cells exhibits a tenoblast-like phenotype. In other embodiments, the implant comprises at least one tendon neotissue produced after the tenogenic differentiation of a population of ASCs into a population of tenocytes-like cells expressing at least one tendon-specific marker gene.

In certain embodiments, an implant may be used for treating a tendon and or/ligament injury in a mammalian subject. In preferred embodiments, the subject is an equine. In one aspect of the embodiment, the implant may be supplemented with additional ECM components including collagen, laminin, fibronectin, PLA, PGLA, PLLA, PEEK, PEG, clastin, tenomodulin, and fibromodulin.

Tendon injuries may be characterized by decreased collagen fiber organization, increased cellularity with fibroblasts and infiltrating endothelial cells or inflammatory cells such as monocytes or macrophages.

In some embodiments, the implant of tendon neotissue having a collagen-based scaffold populated with differentiated tenocyte-like cells is implanted on the site of the injury which results in in vivo enhanced tendon neotissue formation that exhibits the physiological and mechanical characteristics of native tendon tissue. In one aspect of the embodiments, the neotendon implant is intended to repair a tendon injury caused by inflammation i.e., tendinitis or by tears in tissue in and around the tendon due to overuse over an extended period of time i.e., tendinosis. The implant produced according to certain embodiments may be used for repair of an injury affecting any tendons and/or ligaments in a subject's body, for example resulting from inflammation or gradual wear and tear due to overuse or aging.

In some embodiments, the implant is utilized to repair acute calcaneal tendon rupture that is often preceded by tendinosis. The neotendon tissue implanted into calcancal tendons formed an intertwined meshwork of collagen fiber bundles that can be infiltrated by endogenous fibroblastic cells. Implantation of a construct in the calcaneal tendon leads to repair of the tendon having good mechanical properties. In a functional tendon neotissue, the collagen fibers and the cells populating the collagen-based scaffold are oriented to effectively control and constrain tensile stress.

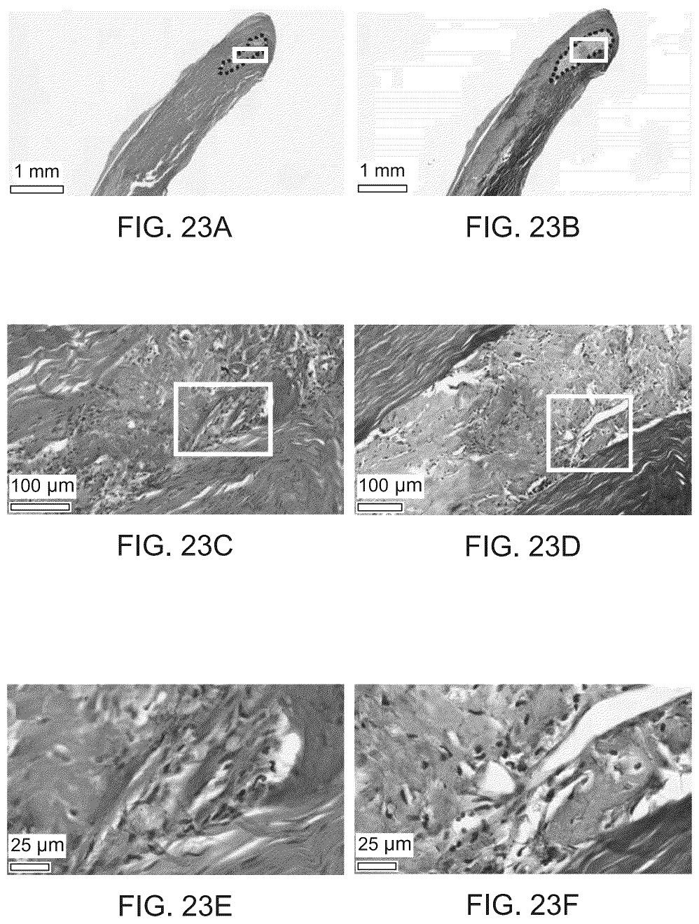

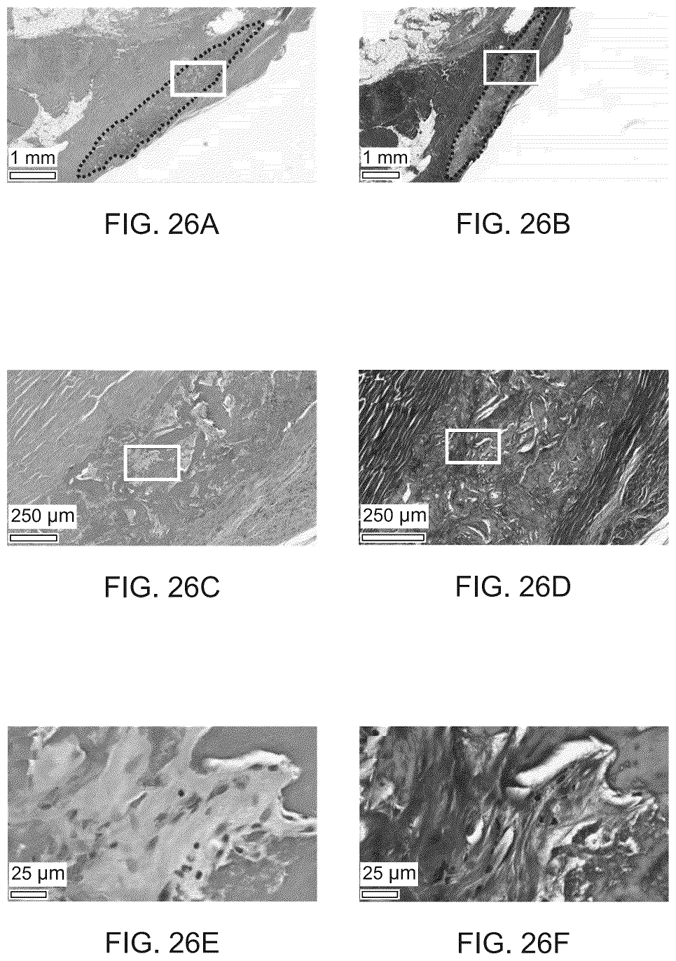

In some embodiments, the ASCs are autologous to the subject receiving the implant which are isolated from that particular subject. In other embodiments, the ASCs are allogeneic to the subject receiving the neotendon implant. In one aspect of the embodiments, the implant is a xenogeneic tendon neotissue derived from xenogeneic ASCs differentiated onto a xenogeneic scaffold. In some embodiments, off-the-shelf allogeneic and xenogeneic implants can be produced and cryopreserved until needed. Allograft and xenograft of tendon neotissue do not induce a significant cell-mediated immune response. (See FIGS. 23F and 26C) An immune response would have included mononuclear cell infiltration and fibrotic encapsulation of the implant.

Tenocytes respond to inflammatory environments and trigger macrophage polarization. M1 polarization of macrophages appeared to be an important component of normal tendon healing, as exosomes from M2 macrophage promote peritendinous fibrosis, a common complication of tendon injuries. In human, increase presence of macrophages are observed in tendinopathic calcaneal tendons.

Regardless of the subject's immune status, implants of tendon neotissue remain morphologically viable and assume orthotopic tendon characteristics. In some embodiments, the implants of tendon neotissue show neovascularization, with the cells and the fibrous ECM align parallel to the surrounding native tissue. (See FIGS. 23D-23F, 26F) In some embodiments, tendon neotissues implanted in injured calcaneal tendon shorten the healing process and decrease stiffness, which underscore their tenoconductive properties. Due to the tenoconductive properties, the tendon neotissues augment primary repair, reduce retear rate, accelerate healing, and improve or restore functions. Collagen-based scaffolds colonized with tenocyte-like cells derived from ASCs and cultured in tenogenic media lead to better fibrocartilage and tidemark formation at the bone-to-tendon interface, increases collagen fiber density, improves fiber orientation, and increases load-to-failure.

Ultrasound is the established imaging modality to identify and monitor healing of equine tendon lesions. In one aspect of the embodiments, implants may be injected into equine tendon lesions with ultrasound guidance. Tendon neotissue stiffness is compatible with injection, so it is feasible that it could be administered with ultrasound guidance following appropriate safety and efficacy testing.

### Bioreactor System

ASCs have great potential for therapeutic applications. In certain embodiments, the methods of inducing tenogenic differentiation in ASCs are performed in a bioreactor system 405 as illustrated in FIG. 4A. The bioreactor system which supports ASCs growth and differentiation within three-dimensional (3D) tissue formation is an important technology for tendon engineering.

In certain embodiments, the bioreactor system 405 for producing a 3D engineered tissue comprises a bioreactor 221 and a perfusion system. (FIGS. 2-4) The bioreactor 221 provides the environmental conditions necessary for a dynamic culture. To differentiate stem cells like ASCs, such dynamic culture provides better control and monitoring of the parameters affecting their growth, proliferation, and differentiation than traditional static 2D culture. Notably, the bioreactor 221 also allows 3D environments that can be designed to mimic stem cell niches.

In certain embodiments, the bioreactor comprises a base chamber 217 that receives the medium and the construct comprising a biopolymer-based scaffold 201 infused with a population of ASCs, a core frame 223 comprising an immobile horizontal bar 211 at the bottom for securing the construct 201 via the lower loop 209, and gas exchange access port 411, a top lid 219 having a medium access port 417, and an adjustable horizontal bar 213 for securing the construct 201 via the upper loop 207, the adjustable horizontal bar 213 is attached to a vertical threaded bar 215 which is secured to the top lid via a nut 216. (FIGS. 2B-2D and 3) The vertical bar 215 can move upward and downward thereby allowing for a change in distance between the immobile horizontal bar 211 and the adjustable horizontal bar 213 to apply a dynamic or static tensile strain to the construct 201. In one aspect of the embodiments, the distance between bottom immobile horizontal bar 211 and top adjustable horizontal bar 213 was set to apply a static and continuous tensile strain of 10% on the cell-scaffold construct during the culture. A dynamic or static tensile strain applied to the construct may have an amplitude of about 1 to 75%. In one aspect of the embodiments, the vertical bar 215 is a threaded bar which can rotate upward to increase the distance between the bottom immobile horizontal bar 211 and the top adjustable horizontal bar 213 such as to increase the tensile strain applied to the construct placed in the base chamber. In another aspect of the invention, the vertical bar 215 can rotate downward to decrease the distance between the bottom immobile horizontal bar 211 and the top adjustable horizontal bar 213 such as to decrease the tensile strain applied to the construct. In the base chamber 217, the construct is submerged in medium where it is further subjected to sheer stress induced by the flow of the medium produced by a perfusion system.

In certain embodiments, the culture parameters in the bioreactor are controlled by a perfusion system which delivers a flow of medium submerging the construct 601. (FIG. 4A) The perfusion system offers many benefits such as fewer mass transfer limitations and mechanical stimulation. In one aspect of the embodiments, the perfusion system includes a medium reservoir 409 containing medium, preferably a tenogenic medium containing at least one differentiation driver that can induce and maintain the differentiation process. The reservoir also contains a deformable membrane for facilitating gas exchange with the base chamber 217. Gas exchange is facilitated via gas exchange access port 411 connected by a gas exchange conduit 421 between the gas exchange access port of the reservoir 412 and the base chamber 217 of the bioreactor 221. A peristaltic pump 401 connects independently to the medium reservoir 409 and to the bioreactor 221 and controls the rate and directionality of the flow of medium in and/or out of the bioreactor 221. The peristaltic pump 401 may be controlled by a computer. The flow of medium produced by the perfusion system ensures the delivery of nutrient, gas exchange and movement of mass products which enhances the viability and the functions of the cells in culture. In one aspect of the embodiments, the rate of the perfusion flow is between 2 and 50 ml/minutes, preferably 10 ml/minute.

In addition, to dispersing nutrients and waste products in the bioreactor 221, the perfusion system produces a bidirectional perfusion flow and a centrifugal flow motion, thereby inducing flow shear stress onto the construct. The perfusion system further comprises an agitator 407 that creates the centrifugal flow motion thanks to a magnetic stir bar 403 placed in below the immobile horizontal bar 211, enhancing mass transport, and nutrients and gas diffusion. An example of agitator includes, but is not limited to, a magnetic stirrer 407 which utilizes a rotating magnetic field causing a magnetic stir bar 403 within the bioreactor 221 (see FIG. 3B) to spin, thereby creating a centrifugal flow motion. In one aspect of the embodiments, the centrifugal flow motion has a speed of between 10 to 10,000 rpm, preferably 300 rpm. The bioreactor system is maintained in a thermoregulated CO₂ incubator.

In some embodiments, the bioreactor system 405 is used to induce the tenogenic differentiation of a population of ASCs which is seeded onto a collagen-based scaffold 201 to form a cell-scaffold construct 601 subjected to biochemical cues (e.g., tenogenic differentiation driver) and mechanical loadings (sheer stress and dynamic or static tensile strain) to produce a population of tenocytes-like cells embedded onto the scaffold and ECM components to form tendon neotissues.

The bioreactor system 405 of the embodiments is amenable to multiple engineered construct productions, 3D engineered tissue development, and yet is simple to operate and can be scaled up for potential clinical uses. While the bioreactor system is utilized to engineer 3D tendon neotissue from ASCs and implement tenogenic differentiation, it can be simply adapted to other types of cells which require mechanical loadings to proliferate, grow and/or differentiate.

### EXAMPLES

1. De Novo Tendon Neotissue from Equine Adult Stem Cells

### 1.1. Materials and Methods

### 1.1.1. Study Design: Neotendon Culture Using ASCs and COLI Template

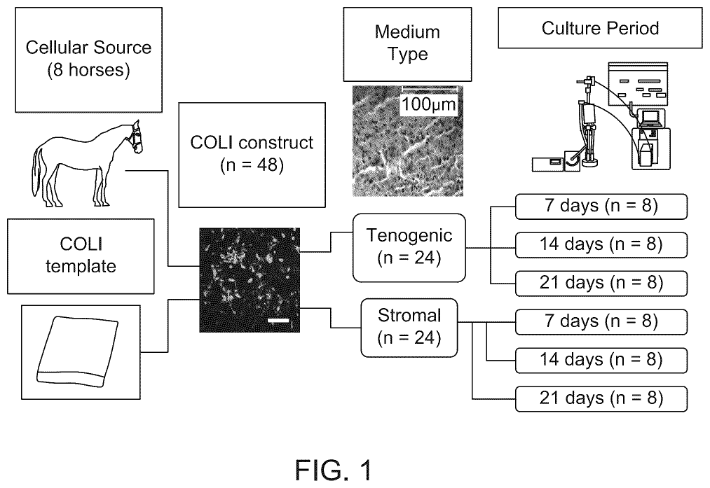

ASCs were seeded onto a COLI template and cultured in tenogenic or stromal medium for up to 21 days under 10% static strain. Supragluteal ASCs, passage 2, from 8 adult horses (4 geldings, 4 mares, body condition score 4-7, 5-21 years, 425-500 kg) were seeded at 1.0×10⁶ cells/cm³ onto bovine COLI sponge (n=48), each sizing 6.0×4.0×1.0 cm³, after rolled into a cylinder of commercially available bovine COLI (Aitene™ Ultrafoam™ Collagen Sponge, Davol Inc., Warwick), and wrapped with a finger trap suture 205. Half of the constructs were cultured in stromal medium (n=24) and half were cultured in tenogenic medium (n=24) using custom-designed bioreactors for 7 (n=8 for each medium), 14 (n=8 for each medium), and 21 (n=8 for each medium) days under 10% static strain (FIG. 1).

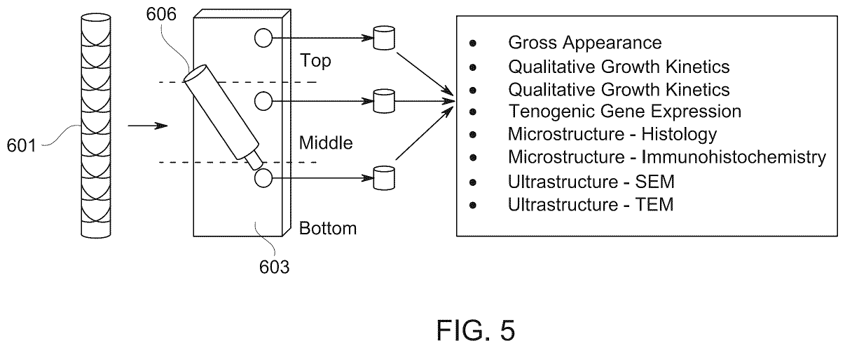

Gross appearance of constructs was documented with digital imaging prior to sample collection from the upper, middle, and lower regions of the construct relative to bioreactor orientation (see FIG. 5) for outcome measures. Qualitative and quantitative cell growth kinetics, tendon-specific gene expression of constructs were evaluated with calcein-AM/EthD-1 staining, resazurin reduction, and RT-PCR, and light—as well as transmission and scanning electron microscopy. Microstructure of constructs was evaluated histologically with hematoxylin and eosin (H & E) staining and immunohistochemical by staining against fibromodulin deposition. Fibromodulin is a key regulator of tendon fibril maturation, and it is most prevalent in the later stages of embryonic fibrillogenesis. Ultrastructure of constructs was evaluated with SEM and TEM.

Additionally, the effect of dynamic strain on construct culture was evaluated. To apply dynamic strain, one end of each construct was continuously displaced at 10% strain in a sine wave at 1 Hz frequency during culture in tenogenic medium for 21 days. Specimens were harvested and evaluated histologically with H & E staining and ultrastructurally with SEM.

### 1.1.2. Equine Subcutaneous Adipose Tissue-Derived Multipotent Stromal Cells (ASCs)

Equine subcutaneous adipose tissue was aseptically harvested from the supragluteal region of 4 adult geldings and 4 mares euthanized for reasons unrelated to this study immediately post-mortem. About 45 mL of subcutaneous adipose tissue was aseptically harvested via sharp dissection from the supragluteal region. Donor inclusion criteria were: 1) 425-500 kg; 2) 5-21 years; 3) no acute or chronic systemic illness; 4) mares or geldings. The stromal vascular fraction was isolated as previously described within 2 h of harvest. Briefly, adipose tissue was minced and mixed with an equal volume of phosphate buffered saline (PBS, PBS 1×, Thermo Fisher Scientific). The mixture was allowed to separate into two phases, and the infranatant was digested for 2 h at 37° C. in an equal volume of PBS with 1% bovine serum albumin (BSA, Sigma Aldrich, Co, Saint Louis, MO) and 0.1% type I collagenase (Worthington Biochemical, Lakewood, NJ) in PBS. After addition of 1% BSA, the mixture was centrifuged (2.6×10² g, 5 min, 4° C.). The resulting stromal vascular fraction pellet was resuspended in PBS and centrifuged (2.6×10² g, 5 min). The pellet was resuspended in stromal medium (Dulbecco's modified eagle medium (DMEM)-Ham F12 (HyClone Laboratories, LLC, Logan, UT), 10% fetal bovine serum (FBS, HyClone Laboratories), 1% antibiotic/antimycotic solution (HyClone Laboratories). Viable cell numbers were quantified with methylene blue (methylene blue hydrate, Sigma Aldrich) staining and a hemocytometer (Hausser Scientific™ Bright-Line™ Counting Chamber, Fisher Scientific).

Isolated cells were cultured in 10 cm culture dishes (CellStar®, VWR, Radnor, PA) at 5×10³ cells/cm² with stromal medium that was refreshed after 24 h and then every 2-3 days (5% CO², 37° C., 90% humidity) ASCs were maintained in stromal medium (DMEM-Ham's F12, 10% FBS, 1% antibiotic/antimycotic) until 80% confluence followed by cryopreservation at passage 0 (P0) using a conventional protocol.

Cryopreserved ASCs were revitalized and expanded to P1 in stromal medium prior to construct culture in tenogenic (DMEM-high glucose, 1% FBS, 10 ng/ml transforming growth factor (TGF)-β1, 50 μM L-ascorbic acid 2-phosphate sesquimagnesium salt hydrate, 0.5 μg/ml insulin, 1% antibiotic/antimycotic) or stromal medium (Dulbecco's modified Eagle medium (DMEM)-Ham F12 (Hyclone Laboratories), 10% fetal bovine serum (FBS, HyClone Laboratories) at P2.

### 1.1.3. Bioreactor System

Templates were composed of a commercially available COLI sponge consisting of a partial hydrochloric acid salt of purified bovine corium (Avitene™ Ultrafoam™ Collagen Sponge, Davol Inc.) that is approved for use as microfibrillar collagen hemostat by the US Food and Drug Administration. The sponge is porous, pliable, water insoluble and bioabsorbable, and it is produced by lyophilization of a slurry of water and purified collagen. The manufacturing process permits noncovalent attachment of hydrochloric acid to amine groups on the collagen molecules and preserves their native morphology.

For each template, a COLI sponge section 202 (Avitene™ Ultrafoam™ Collagen Sponge, Davol Inc., Warwick, RI), 6.0×4.0×1.0 cm³ (length×width×height), was rolled into a column with a diameter of 1.0 cm and length of 6.0 cm. The sponge section 202 was wrapped by a finger trap composed of polydioxanone suture 205 (PDS® II, Ethicon, Somerville, NJ) with 1 cm long loops 207, on each end for securing the scaffold 201 to the immobile horizontal bar 211 and adjustable horizontal bar 213 within a bioreactor 221. The bioreactor 221 illustrated in FIGS. 2B-3 was a custom design by the inventors that was produced by stereolithography out of liquid resin (SOMOS® WaterShed® XC 11122, Stratasys, Waltham, MA) by a commercial printing company (Proto Labs, Inc., Maple Plain, MN). A core frame 223 with an immobile cross beam 211 on the lower end of a core frame 223 fit within the base chamber 217 measuring 80×45 mm (height×diameter). The bioreactor top lid 219 was secured to the top of the base chamber 217 with matching threads. A threaded, vertically adjustable cross beam 215 attached to the upper lid with matching threads extended downward into the core frame 223 within the base chamber 217. The distance between the adjustable bar 213 and the immobile horizontal bar 211 on the core frame 223 was adjusted by turning the vertical threaded bar 215 to advance the mobile bar toward or away from the top lid 219, and the position was fixed with a nut 216 on the top of the lid. The space between the nut and the lid was sealed with sterile rubber gasket material.

Cell-scaffold constructs 601 were secured with the suture loops 207, 209 on each end of the construct. (FIG. 2D) One suture loop 209 is secured to the immobile horizontal bar 211 at the bottom of core frame 223 (shown in FIGS. 2B and 3). The other suture loop 207 is secured to the adjustable horizontal bar 213 near top of bioreactor thereby applying a static strain to the construct during the culture period (Sec FIGS. 2C and 3). The distance between the bars was initially set at the correct distance for a 10% construct strain based on the length of the construct with no tension applied as determined with an electronic caliper (Mitutoyo #500-196, Mitutoyo Corp., Japan). When medium was exchanged every several days, for example every 7 days, the height of the adjustable horizontal bar 213 was adjusted if necessary to maintain 10% strain based on the construct length determined at that time by turning the vertical threaded bar 215. A magnetic stir bar 403 (2.5×0.7 cm) was placed in the chamber base chamber 217 beneath the immobile bar 211 of the core frame 223. The bioreactor 221 consisted of top lid 219, core frame 223, and base chamber 217. An immobile horizontal bar 211 is located inside core frame 223 and an adjustable horizontal bar 213 is attached to the top lid 219 via vertical threaded bar 215 (FIG. 3). The distance between bottom immobile horizontal bar 211 and top adjustable horizontal bar 213 was set at 6.6 cm to apply 10% strain on the Cell-scaffold construct 601 during culture period.

The bioreactor 221 was connected to a perfusion system that consists of medium reservoir 409, peristaltic pump 401, and medium stirrer 407 (FIG. 4A). The lowest port 411 of each bioreactor was attached to a gas exchange port 412 of a 10 ml medium reservoir 409 (Synthecon, Houston, TX) with gas exchange conduit/tubing 413 (4.8 mm inner diameter: Tygon®, Compagnie de Saint-Gobain, Courbevoie, France). The reservoir 409 had an oxygenator membrane at the bottom side for gas exchange. The medium access port 417 on bioreactor cap and a port of medium reservoir 409 were connected to a computer-controlled pcristaltic pump 401 (ISM404b, Ismatec, Wertheim, Germany) via 3-way stopcocks 415 attached to 0.22 μm microfilters 423 to which medium conduit/tubing 421 (1.0 mm inner diameter: Compagnie de Saint-Gobain) between the bioreactor 221 and the pump 401 were attached.

Medium perfusion was driven by a computer-controlled peristaltic pump 401 (Ismatec model ISM404b, Huiyu Weiye (Beijing) Fluid Equipment Co., Ltd., Beijing, China) connected to the bioreactor 221 and medium reservoir 409 (FIG. 4). The pump was separately attached with tubing 421 (1.0 mm inner diameter; Tygon®, Compagnie de Saint-Gobain) to a 0.22 μm sterile syringe filter 423 (MilliporeSigma™, Thermo Fisher Scientific) which was connected to another segment of tubing 421 (4.8 mm inner diameter; Tygon®, Compagnie de Saint-Gobain) attached to one port on the upper surface of a 10 mL medium reservoir 409 (High Aspect Ratio Vessel, Synthecon, Inc., Houston, TX). Another port on the upper surface of the medium reservoir 409 was attached to the lower gas exchange access port 411 of the bioreactor 221 with tubing 413 (4.8 mm inner diameter; Tygon®, Compagnie de Saint-Gobain). The reservoir 409 provided medium oxygenation via a flat gas transfer membrane, for example a silicon rubber gas transfer membrane.

System fluid flow rate was computer controlled (Lab View™, National Instruments, Austin, TX) at 10 ml/minute, and direction reversed before medium reached one side of microfilter. The direction of fluid flow was reversed when it reached the upper and lower ends of the tubing between the bioreactor lid 219 and the syringe filter 423. Centrifugal medium motion within the bioreactor was generated with the magnetic stir bar 403 beneath the core frame 223 in the chamber (300 rpm) that was driven by a stir plate or agitator 407 (Isotemp™, Thermo Fisher Scientific) positioned beneath the bioreactor 221. The fluid flow was both ingresss and centrifugal. Fluid flow rate and stir speed were based on progressive iterations of the current system to optimize viable cell distribution in the scaffold 601. All bioreactor system parts were sterilized with ethylene oxide prior to assembly and use. The perfusion system pump was maintained in a CO₂ incubator (5% CO₂, 37° C.) for the duration of the culture period.

The system was used to seed P2 ASCs into COLI templates at 1.0×10⁶ ASCs/cm³ template in stromal or tenogenic medium through the 3-way stopcocks 415 and maintained for 21 days. Specifically, a template was premoistened and added to the bioreactor after the system was filled with stromal or tenogenic culture medium. It was maintained in the incubator for 1 h with fluid motion. Subsequently, the fluid motion was paused. Cells (1×10⁶ cells/cm³ template) were added to the medium of the bioreactor chamber 217 through the 3-way port 415 on the tubing 421 attached to the bioreactor lid 219, and fluid motion was restarted. Medium was exchanged periodically. For a period of 21 days of culture, the medium was changed every 7 days.

Bioreactor system according to one embodiment (FIG. 4A) and cyclic tensioner 419 (FIG. 4B). For construct culture under static strain (FIG. 4A), a bioreactor 221 was attached to a 10 ml medium reservoir 409 for gas exchange, both of which were connected to a computer-controlled peristaltic pump 401 via 3-way stopcocks 415 through 0.22 μm microfilters 423 between the bioreactor 221 and the pump 401. Additional medium perfusion was rendered via stirrer 403. In an alternative embodiment for placing construct culture under dynamic strain (FIG. 4B), a taller bioreactor 221 allowed vertical motion of stirrer attached to the bottom of construct by cyclic tensioner 419 externally applying magnetic field, while stirrer at the bottom of the bioreactor chamber was stirred. Cyclic tensioner 419 was driven by vessel rotator.

### 1.1.4. COLI Construct Specimen Harvest

As illustrated in FIG. 5, once each culture period was reached (e.g., after 7, 14 or 21 days of culture), cell construct 601 was harvested from the bioreactor 221. Suture 205 were removed, construct unrolled 603, and full thickness cylindrical specimens were collected with a biopsy punch 605 to collect full thickness samples from each region (diameter 4.0 mm) from the top/highest, middle and bottom/lowest regions of the long axis of each construct. A total of three specimens from each region was allocated to each outcome. Differences in cell viability staining among harvest regions were assessed. Remaining portions of each construct sample after collecting cylindrical specimens for other outcome measures was used as a single representative specimen combining all 3 top, middle, and bottom regions for tenogenic gene expression measurement.

### 1.1.5. Qualitative and Quantitative Growth Kinetics

To evaluate qualitative growth kinetics of cells within constructs, specimens were stained with calcein acetoxymethyl (calcein-AM: Thermo Fisher, Waltham, MA) for viable cells and ethidium homodimer-1 (EthD-1: Thermo Fisher) for nonviable cells both at the concentrations of 4.0 μM in PBS at 37° C. for 30 minutes. After incubation with calcian-AM and EthD-1, specimens were briefly washed with PBS, placed on glass slide, and pressed down with cover glass to make thin section. The microstructural distribution of viable and nonviable cells as well as cellular morphology within entire specimen were imaged with confocal laser microscope (TCS SP8: Leica, Wetzlar, Germany) at multiple focal planes across full thickness of sample.

To evaluate quantitative growth kinetics of cells within construct, the number of viable cells in each specimen was indirectly quantified using cellular metabolic activity measured by incubating with 100 μl of 50 μM resazurin reduction (Thermo Fisher) at 37° C. for 3 hours. After incubation, resazurin mixture was collected, of which 50 μl was mixed with 50 μl of PBS, and resorufin fluorescence measured at an excitation wavelength of 540 nm and an emission wavelength of 590 nm using microplate reader (SPARK® Multimode Microplate Reader: TECAN, Männedorf, Switzerland). Resazurin solution incubated without specimen was used as negative control to subtract fluorescence from the sample values.

### 1.1.6. Gene Expression-Reverse Transcription Polymerase Chain Reaction (RT-PCR)

Remaining portions of each COLI construct sample after collecting cylindrical specimens for other outcome measures was used as a single representative specimen for each COLI construct sample combining all 3 top/highest, middle, and bottom/lowest regions. Specimens were digested at 37° C. in 0.1% type I collagenase in PBS for 1 hour, spun down at 300×g for 10 minutes at 4° C., and supernatant discarded. One milliliter of TRI Reagent® (Sigma, St. Louis, MO) was added to the precipitate, and homogenized by passing mixture through 18-gauge needle 30 times. Homogenate was spun down at 21,000×g for 15 minutes at 4° C. Total RNA was extracted from supernatant by phenol-chloroform extraction according to the manufacturer's instructions. Isolated RNA was cleaned up by RNeasy® Mini Kit (QIAGEN, Hilden, Germany). One microgram of total RNA was used for cDNA synthesis (QuantiTect® Reverse Transcription Kit, QIAGEN).

Equine-specific primers for tendon-specific genes, scleraxis (Scx) (SEQ ID NO: 1 and SEQ ID NO: 2), mohawk (Mkx) (SEQ ID NO: 3 and SEQ ID NO: 4), early growth response 1 (Egr1) (SEQ ID NO: 5 and SEQ ID NO: 6), connective tissue growth factor (CTGF) (SEQ ID NO: 7 and SEQ ID NO: 8), lysyl oxidase (LOX) (SEQ ID NO: 9 and SEQ ID NO: 10), collagen 1a1 (Col1a1) (SEQ ID NO: 11 and SEQ ID NO: 12), collagen 3a1 (Col3a1) (SEQ ID NO: 13 and SEQ ID NO: 14), decorin (Dcn) (SEQ ID NO: 15 and SEQ ID NO: 16), elastin (Eln) (SEQ ID NO: 17 and SEQ ID NO: 18), tenascin-c (TnC) (SEQ ID NO: 19 and SEQ ID NO:20), biglycan (Bgn) (SEQ ID NO: 21 and SEQ ID NO: 22), fibromodulin (Fbmd) (SEQ ID NO: 23 and SEQ ID NO: 24), collagen 14a1 (Col14a1) (SEQ ID NO: 25 and SEQ ID NO: 26), and truncated hemoglobin 4 (THBS4) (SEQ ID NO: 27 and SEQ ID NO: 28) were quantified using primers previously validated⁴⁰⁻⁴³ or designed with Primer-BLAST (National Center for Biotechnology Information, Bethesda, MD). (See Table 1) PCR was performed with denaturation step at 95° C. for 15 minutes, followed by 40 cycles of denaturation at 94° C. for 15 seconds, annealing at 52° C. for 30 seconds, and elongation at 72° C. for 30 seconds using SYBR Green system (QuantiTect® SYBR® Green PCR Kits, QIAGEN). Relative gene fold change was determined by standard means (2-ΔΔCt). Glyceraldehyde 3-phosphate dehydrogenase (GAPDH) (SEQ ID NO: 29 and SEQ ID NO: 30) was used as the reference gene.

TABLE 1
Equine-specific Primer Sequences

| | | | | Amplicon | Accession |
|---|---|---|---|---|---|
| Gene | | Sequence (5′ - 3′) | SEQ ID NO: | Length | Number |
| Scx | Fwd | TCTGCCTCAGCAACCAGAGA | 1 | 246 | NM_001105150.1 |
| | Rev | AAAGTTCCAGTGGGTCTGGG | 2 | | |
| Mkx | Fwd | AGTGGCTTTACAAGCACCGT | 3 | 217 | XM_023632371.1 |
| | Rev | ACACTAAGCCGCTCAGCATT | 4 | | |
| Egr1 | Fwd | CCTACGAGCACCTGACCTCAG | 5 | 241 | XM_001502553.5 |
| | Rev | GATGGTGCTGAAGATGAAGTGG | 6 | | |
| CTGF | Fwd | ACCCGCGTTACCAATGACAA | 7 | 140 | XM_023651101.1 |
| | Rev | GGCTTGGAGATTTTGGGGGT | 8 | | |
| LOX | Fwd | CAGGCGATTTGCGTGTACTG | 9 | 301 | XM_023617821.1 |
| | Rev | ACTTCAGAACACCAGGCACT | 10 | | |
| Collal | Fwd | CAAGAGGAGGGCCAAGAAGA | 11 | 261 | XM_023652710.1 |
| | Rev | TCCTGTGGTTTGGTCGTCTG | 12 | | |
| Col3a1 | Fwd | TCCTGGGGCTAGTGGTAGTC | 13 | 255 | XM_008508902.1 |
| | Rev | GGCGAACCATCTTTGCCATC | 14 | | |
| Dcn | Fwd | TTATCAAAGTGCCTGGTG | 15 | 204 | XM_005606467.3 |
| | Rev | CATAGACACATCGGAAGG | 16 | | |
| Eln | Fwd | CTATGGTGTCGGTGTCGGAG | 17 | 247 | XM_023655466.1 |
| | Rev | GGGGGCTAACCCAAACTGAG | 18 | | |
| TnC | Fwd | TACTGATGGGGCCTTCGAGA | 19 | 330 | XM_023628745.1 |
| | Rev | AGCAGCTTCCCAGAATCCAC | 20 | | |
| Bgn | Fwd | TGATTGAGAACGGGAGCCTGAG | 21 | 143 | XM_023633175.1 |
| | Rev | TTTGGTGATGTTGTTGGTGTGC | 22 | | |
| Fbmd | Fwd | GCTTCTGCTGAGGGACAC | 23 | 91 | NM_001081777.1 |
| | Rev | GATTTCTGGGGTTGGGAC | 24 | | |
| Col14a1 | Fwd | CTGGACGATGGAAGTGAG | 25 | 215 | XM_005613197.3 |
| | Rev | GTGACCCTGAACTGCTGC | 26 | | |
| THBS4 | Fwd | ACGTAAACACCCAGACGGAC | 27 | 359 | XM_023618094.1 |
| | Rev | CACCAACTCGGAGCCTTCAT | 28 | | |
| GAPDH | Fwd | GTGTCCCCACCCCTAACG | 29 | 131 | NM_001163856.1 |
| | Rev | AGTGTAGCCCAGGATGCC | 30 | | |

### 1.1.7. Histological Microstructure

Specimens were fixed in 4% paraformaldehyde (PFA) overnight at 4° C., serially dehydrated in increasing concentration of ethanol and xylene, paraffin embedded, and sectioned (5 μm). Sections were deparaffinized in xylene, and serially rehydrated in decreasing concentrations of ethanol, followed by staining with haematoxylin and eosin (H & E). H & E staining was performed with incubation of sections with hematoxyline at room temperature for 3 minutes followed by washing with deionized water and tap water. Sections were then incubated with eosin at room temperature for 30 seconds, serially dehydrated with increasing concentrations of ethanol and xylene, mounted with mounting medium (Permount™ Mounting Medium: Thermo Fisher) and cover glass.

Cellular morphology, distribution, and extra cellular matrix deposition were evaluated after digital images generated with a slide scanner (NanoZoomer, Hamamatsu Photonics K.K, Hamamatsu City, Japan) or a light microscope (DM4500B, Leica, Wetzlar, Germany) fitted with a digital camera (DFC480, Leica).

### 1.1.8. Immunohistochemical Microstructure

The same paraffin blocks prepared for histological microstructural analysis were sectioned (5 μm), deparaffinized in xylene, and serially rehydrated in decreasing concentrations of ethanol, followed by incubation in PBST (0.1% Triton X-100 in PBS) at room temperature for 10 minutes. Antigen-retrieval was performed in antigen retrieval buffer (100 mM Tris, 5% Urea, pH 9.5) at 121° C. for 30 minutes using autoclave. Specimens were incubated in blocking buffer (1% BSA and 22.52 mg/ml glycine in PBST) at room temperature for 30 minutes. Sections were stained with rabbit anti-human fibromodulin (PA5-26250: Invitrogen, Waltham, MA) polyclonal antibody at concentration of 1:100 in incubation buffer (1% BSA in PBST) overnight at 4° C. Sections were washed with PBS at room temperature for 15 minutes each 3 times, then stained with goat anti-rabbit IgG conjugated with Alexa Fluor™ 488 (A11070: Molecular Probes, Eugene, OR) at concentration of 1:200 in incubation buffer for 1 hour at room temperature. Following washing with PBS 3 times, nucleus was counter-stained with 4′,6-diamidino-2-phenylindole (DAPI: Thermo Fisher) at concentration of 10 μM in PBS at room temperature for 10 minutes. Sections were washed with PBS once and mounted with mounting medium (Vectashield® Antifade Mounting Medium: Vector Laboratories, Newark, CA) and cover glass. Images were obtained at an excitation wavelength of 490 nm and an emission wavelength of 525 nm using confocal microscope (TCS SP8: Leica). Sections stained with only secondary antibody was used as negative control, and sections of equine DDFT was used as positive control.

### 1.1.9. Scanning Electron Microscopy (SEM)

Specimens were fixed in 2% PFA and 1.25% glutaraldehyde in 0.1 M sodium cacodylate (CAC) buffer (pH 7.4) for 1 hour at room temperature and transferred to buffer (3% glutaraldehyde in 0.1 M CAC buffer, pH 7.4) for 30 minutes. They were rinsed with washing buffer (5% sucrose in 0.1 M CAC buffer, pH 7.4), post-fixative buffer (1% osmium tetroxide in 0.1 M CAC buffer, pH 7.4), and water. Specimens were serially dehydrated, critical point dried, and sputter coated with gold. Digital images were created with a scanning electron microscope and camera at 15 kVp (Quanta 200, FEI Company, Hillsboro, OR).

### 1.1.10. Transmission Electron Microscopy (TEM)

Specimens were collected from constructs cultured in stromal and tenogenic medium both for 21 days. Specimens were fixed in 2% PFA and 2% glutaraldehyde in 0.1 M PBS (pH 7.4) at 4° C. overnight. They were washed 3 times in 0.1 M PBS for 30 min each, and post-fixed in 2% osmium tetroxide (OsOa) in 0.1 M PBS at 4° C. for 3 hours. Specimens were dehydrated in grading ethanol, infiltrated with propylene oxide twice for 30 minutes each, and were placed in a 70:30 mixture of propylene oxide and resin for 1 hour, followed by polymerization in 100% resin at 60° C. for 48 hours.

The polymerized resins were sectioned at 70 nm with a diamond knife using an ultramicrotome (Ultratome Leica EM UC7: Leica), mounted on copper grids, and stained with 2% uranyl acetate at room temperature for 15 minutes. They were stained with lead stain solution at room temperature for 3 minutes. Images were obtained with a transmission electron microscope (JEM-1011, JEOL Ltd., Tokyo, Japan) at an acceleration voltage of 80 kV.

### 1.1.11. Statistical Analysis

Results are presented as mean+standard error of the mean (SEM). Normality of data was examined with the Kolmogorov-Smirnov test. Outcome measures were compared with ANOVA. When overall difference was detected, pairwise comparisons between groups were performed using Tukey's post-hoc test. Fold changes of tenogenic gene expression of COLI constructs cultured in tenogenic medium normalized to those cultured in stromal medium for identical periods were compared using one sample t-test for normally distributed results and Wilcoxon signed rank test for non-normally distributed results. All analyses were conducted using Prism (GraphPad Software Inc., San Diego, CA) with significance considered at p<0.05.

### 1.2 Results

### 1.2.1. Construct Gross Appearance

After each culture medium and period cell-scaffold construct 601 was harvested from bioreactor, rinsed in PBS thoroughly, and gross appearance imaged prior to the specimen collection. In stromal medium, the size of constructs did not change much after 7 days of culture evidenced by construct bulging between finger trap suture loops (FIG. 6A), like dry templates (FIG. 2A). Notably, the template material alone does not expand when moistened. After 14 days, there was no appreciable change in size, but the surface of the lower half of constructs cultured in stromal medium was lighter in color and roughened compared to the upper half (FIG. 6B). After 21 days of culture, the upper half of the constructs were a tan color, the lower half an off-white color, and construct material was frequently missing at the lowest end (FIG. 6C). Constructs cultured in tenogenic medium contracted after 7 days of culture based on loose finger trap suture loops. The constructs were a tan color with occasional red patches and surfaces were roughened (FIG. 6D). After 14 days of culture in tenogenic medium, the constructs were a similar color to after 7 days but had a smooth surface and had contracted more (FIG. 6E). The constructs contracted such that the suture loops rarely contacted the template, more so in the upper half, were a solid tan color, and had an even smoother surface after 21 days of culture (FIG. 6F). Additionally, the constructs were less compressible, and the template layers more tightly adhered than those cultured in stromal medium for 21 days or uncultured template material. Gross images were obtained after harvesting constructs from bioreactor and washing with PBS to remove coloration from medium. Coloration of constructs stems from each medium color. The upper area of each image corresponds to the top in the longitudinal axis of construct during culture.

### 1.2.2. Qualitative Growth Kinetics

Most cells remained viable in both stromal and tenogenic media throughout the culture period with a majority of cells stained with calcein-AM and a few stained with EthD-1 (FIG. 7). The majority of cells were spherical in stromal medium for all culture periods and in tenogenic medium at day 7. Many viable cells assumed a spindle-shape and parallel alignment in tenogenic medium at days 14 and 21 of culture. Viable cells were frequently in clusters in tenogenic medium while individual cells were prevalent in stromal medium.

Cell numbers appeared to have been same in stromal throughout the culture period, whereas cells appeared have proliferated in tenogenic medium from day 7 to 14, and slightly decreased from day 14 to 21.

FIG. 7 depicts cellular morphology and distribution in COLI templates under static strain. Constructs were cultured in stromal (top row) or tenogenic (bottom row) medium for 7 (left column), 14 (middle column), and 21 (right column) days. Viable cells were stained with calcein-AM (green) and non-viable cells were stained with ethidium homodimer-1 (EthD-1, red). Cells were spherical (grey arrows) or elongated (black arrows). Each area indicated with arrow was enlarged in an inset of top right corner. Scale bars=100 μm.

### 1.2.3. Quantitative Growth Kinetics

The bottom region of constructs appears to have more cells regardless of medium type or culture period. The second most populated region of constructs appeared to be the top region of constructs, followed by the middle region. In fact, there was large disparity between specimens heavily populated and those without cells. Therefore, cellular numbers from each region were combined to represent the entire constructs cell numbers.

Differences in the relative fluorescence units among time points for each culture medium were not significant, so time points were combined. The relative fluorescence units of constructs cultured in tenogenic was significantly higher than in stromal medium with all time points combined (p<0.0001). (FIG. 8). Fluorescent intensity of resorufin (mean Â±SEM) in constructs cultured in stromal or tenogenic medium with all time points combined. A difference between culture medium columns is indicated by an asterisk (p<0.05, FIG. 8).

### 1.2.4. Gene Expression-RT-PCR

Throughout the culture period, all tenogenic genes tested had a trend of upregulation in COLI constructs cultured in tenogenic medium compared to those cultured in stromal medium.

Among the transcription factors, Mkx levels were lower (0.75±0.05-fold, p<0.0001) after 14 days and higher (2.28±0.46-fold, p=0.03) after 21 days, respectively, in constructs cultured in tenogenic medium (FIG. 9A). The Egr1 (7.31±1.71-fold, p=0.008) and LOX (86.96±78.50-fold, 0.03) were higher after 7, and CTGF after 7 (5.48±2.47-fold, p=0.03), 14 (20.4±8.76-fold, p=0.02), and 21 (7.69±2.50-fold, p=0.008) days of culture. Among tenogenic genes, mRNA levels of Col1a1 (2.78×103±2.63×103-fold, p=0.02), Col3a1 (2.98×101±2.08×101-fold, p=0.008), Eln (3.03×102±1.44×102-fold, p=0.008), and Bgn (8.52±3.03-fold, p=0.02) were higher after 7 days of culture; Col3a1 (2.62×101±1.59×101-fold, p=0.03), Eln (4.63×102±3.65×102-fold, 0.05), and TnC (7.29±2.40-fold, 0.03) were higher after 14 days of culture; and TnC mRNA levels were higher after 21 (5.12±1.35-fold, p=0.03) days of culture (FIG. 9B). The mRNA levels of mature tendon markers, Fbmd (9.75±4.68-fold, p=0.02) and Col14a1 (2.10×101±1.1×101-fold, p=0.04) were higher after 7 days, and THBS4 (5.85×101±3.55×101-fold, p=0.03) after 14 days of culture (FIG. 9C).

### 1.2.5. Histological Microstructure

Photomicrographs of COL1COL1-ASC constructs cultured in stromal (FIGS. 10A, C, E) or tenogenic (FIGS. 10B, D, F) medium for 7 (A, B), 14 (C, D), or 21 (E, F) days. Cells (white arrows) within variable amounts of ECM (*) as well as template material (cross-hatched arrows) were apparent. The region surrounding each arrow is enlarged in the inset at the right upper corner. Stain: H&E; Scale bars=50 μm (A-C,E), 100 μm (D,F). Histologically, there was no evidence of cell necrosis or apoptosis in constructs cultured in either medium at any time point based on intact cell and nuclear membranes and little to no cytosolic vacuolation (FIG. 10). Rare, individual spheroid cells containing large nuclei, attached to template COLI fibers, and with little to no surrounding ECM were evident in constructs cultured in stromal medium for 7 and 14 days. (FIGS. 10A, 10C) After 21 days of culture in stromal medium, individual, or clustered, spindle- or rhomboid-shaped cells with small, dense nuclei and scant ECM were evident. (FIG. 10E)

Within constructs cultured in tenogenic medium, there were clusters of spindle-shaped cells with large, round to oblong nuclei that were adhered to template COLI fibers and surrounded by ECM after 7 days of culture. (FIG. 10B) After 14 days of culture, cell density and ECM were higher, most cells had a spindle-shaped morphology with dense, elongated nuclei, and cells were positioned in a more parallel arrangement compared to day 7. (FIG. 10D) By day 21 of culture, closely packed, elongated cells with a dense, rod-like nucleus and surrounded by well-organized ECM were aligned in parallel with each other and template COLI fibers. (FIG. 10F) The deposition of ECM also advanced and many areas of COLI template had its pore filled with tissue-like structure. The clusters of cells also tended to form parallel alignment along adjacent collagen fibers. (FIG. 10F).

### 1.2.6. Immunohistochemical Microstructure

The deposition of tenogenic maturation marker protein fibromodulin appeared to be absent in constructs cultured in stromal medium both at day 7 and 14 of culture (FIGS. 11, left panels). After 21 days of culture in stromal medium, however, there appeared to be minimum amounts of fibromodulin deposition throughout the constructs. (FIG. 11, left second bottom panel) Of note was fibromodulin distribution was not consistent with cellular distribution, suggesting fibromodulin was deposited onto COLI template itself rather than the protein existed in the cytosol of cells. Consistent with the other outcome measures, the distribution of cells represented by nucleus stained with DAPI was not homogeneous, and they formed several clusters within constructs.

Similar to constructs cultured in stromal medium at day 7 and 14 (FIGS. 11, left panels), those cultured in tenogenic medium for 7 days had no deposition of fibromodulin onto COLI template. (FIG. 11, right top panel) On the contrary, after 14 days of culture in tenogenic medium, there was progressively increasing deposition of fibromodulin onto COLI constructs. Fibromodulin deposition was moderate and distributed throughout the constructs regardless of cellular distribution. Cells were non-uniformly distributed within constructs, forming clusters represented by aggregates of nucleus. (FIGS. 11, right second top/bottom panels) The deposition of fibromodulin further increased from day 14 to day 21 in tenogenic medium, resulting in extensive amounts of fibromodulin deposited throughout the constructs by day 21. (FIG. 11, right second bottom panel) (Distribution pattern of fibromodulin appeared to be more abundant in the areas where the cells did not form clusters than those immediately adjacent to the cell clusters. And there appeared to be parallelly aligned fibrous structure of fibromodulin deposited areas in the constructs at 21 days, suggesting cells deposited fibromodulin directly onto collagen fibers of COLI template. (FIG. 11, right second bottom panel) Interestingly, the deposition of fibromodulin was even greater in the constructs cultured in tenogenic medium for 21 days than native equine DDFT tissue.

Constructs were cultured in stromal (FIG. 11, left panels) or tenogenic (FIGS. 11, right panels) medium for 7 (FIGS. 11, top panels), 14 (FIGS. 11, second top panels), and 21 (FIGS. 11, second bottom panels) days. Secondary antibody only section (2° Ab only) was used for negative control (−ve ctrl) and DDFT section was used for positive control (+ve ctrl) both at the bottom panels. Green color represents fibromodulin (Fbmd) staining and blue color represents nucleus (DAPI) staining. Scale bars=100 μm

### 1.2.7. Scanning Electron Microscopy (SEM)

Ultrastructural characteristics of cells using SEM indicated cells were spherical to rhomboid within stromal medium cultured constructs throughout the culture period (FIGS. 12A-C). Additionally, they deposited minimum amounts of ECM onto COLI template. Consistent with histological microstructure, cells attached to COLI template with mesh-like ECM deposited around themselves (FIG. 12B). Constructs cultured in tenogenic medium appeared to have small, round cells attached in clusters to template and minimal ECM after 7 days of culture. In contrast, elongated cells that were tightly adhered to collagen template within some ECM were apparent after 14 days, and after 21 days, numerous, rhomboid cells, covered by ECM were tightly adhered to template fibers (FIGS. 12D-12F). Ultrastructure of constructs were cultured in stromal (FIGS. 12A-C) or tenogenic (FIGS. 12D-F) medium for 7 (FIGS. 12A-D), 14 (FIGS. 12B-E), and 21 (FIGS. 12C-F) days and images obtained with SEM. Cells (white arrows) were found surrounded by newly deposited ECM (cross-hatched arrows) on collagen fibers of original template (grey arrows). Scale bars=40 μm (A and F), 50 μm (B, C, and E), 100 μm (D).

### 1.2.8. Transmission Electron Microscopy (TEM)

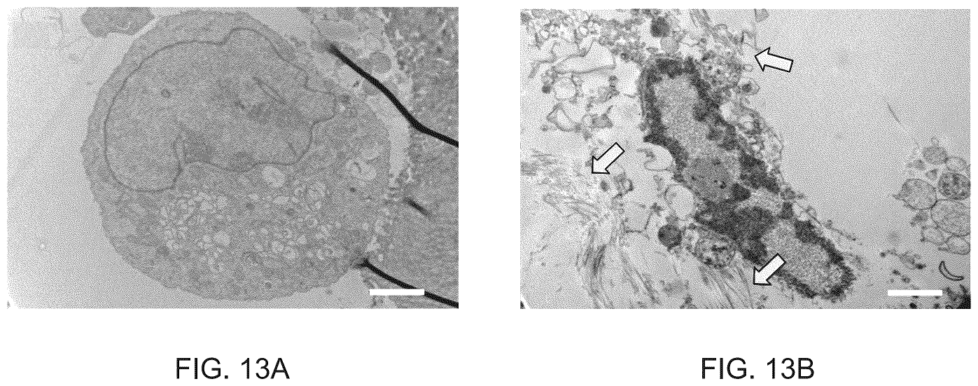

Ultrastructural characteristics imaged with TEM indicated cells were spherical and loosely attached to collagen fibers of COLI template in stromal medium cultured constructs (FIG. 13A). Cells maintained integrity of both plasma membrane and nucleus membrane and were viable. They were rich in mitochondria and Golgi apparatus-like structures. Nucleus to cytosol ratio was relatively high. Lack of ECM-like structures around the cells indicated that they did not actively deposited ECM on existing original COLI template fibers.

In tenogenic medium cultured constructs, cells assumed more elongated morphology represented by elongated rod-like shape of nucleus (FIG. 13B). Nucleus membrane appeared to have maintained integrity, suggesting viability of the cells. The most significant difference between the cells cultured in tenogenic medium and those cultured in stromal medium was the presence of collagen fiber-like structures in their cytosol (FIG. 13B, white arrows). The presence of collagenous fibers in the cells shows a tenocyte-like phenotype. The heterochromatin appears more abundant than in stromal medium-cultured cells, suggesting that transcription activity was relatively less than in tenogenic medium-cultured cells. Constructs were cultured in stromal (FIG. 13A) or tenogenic (FIG. 13B) medium for 21 days and images obtained with TEM. Cells contained collagenous fibers (white arrows) within cells. Scale bars=2 μm (A) and 1 μm (A).

### 1.2.9. Dynamic Strain Culture

Both qualitative and quantitative growth kinetics revealed significantly diminished cellularity within constructs, which resulted in non-detectable level of cellular number by resazurin reduction. Microstructurally, cells were round and formed clusters with surrounding granulofilamentous ECM in tenogenic medium at day 21 (FIGS. 14A-14B). Compared to the constructs cultured under static strain, cell cluster numbers were much lower, indicating interruption of cellular attachment to COLI template. Ultrastructurally, spherical cells surrounded by abundant granulofilamentous ECM formed clusters in tenogenic medium at day 21 (FIGS. 15A-15C). Although cluster numbers were fewer than those cultured under static strain, cellular density within clusters was high and deposition of ECM appeared to be abundant entrapping cells between pillars of COLI template fibers.

### 1.3 Discussion

To date, there have been numerous attempts to culture MSCs onto collagenous scaffold and differentiate into tenocytes. The most used collagenous scaffold for such purpose is hydrogel. Cells or cellular aggregates can be embedded in the hydrogels during solidification, after which viable cells adhered to collagenous fibrils and assume elongated morphology. It was also reported both equine MSCs and tendon-derived MSCs formed non-spindle morphology and instead assembled cell clusters in collagen/synthetic peptide hydrogel in stromal medium culture, which was consistent with ASC-scaffold constructs cultured in stromal medium, thereby confirming that a tenogenic medium culture is an essential component of neotendon formulation. However, it has not been reported that MSCs maintain robust proliferative properties in 3D cultures, which was consistent with non-proliferative cells cultured in stromal medium of the present study. Additionally, decellularized tendon scaffolds have been used to culture MSCs. And it demonstrated equine MSCs adhere to scaffold, yet proliferation of cells has not been reported in part due to inherently anti-proliferative tenogenic medium that include TGF-β. Potential reason for robust proliferative capacity of cells in tenogenic medium from the present study is highly expressed CTGF gene consistently upregulated throughout the culture period. In a previous study, incubation with CTGF not only upregulated tenogenic genes in mouse ASCs but also increased proliferation in dose-dependent manner.

Microstructures of constructs revealed progressive maturation of neotendon in tenogenic medium over the 21-day culture period, presented by increasingly elongating morphology of cells that aligned in parallel with surrounding cells and abundant ECMs deposited around the clusters of cells. The findings were unique in that cells appeared have proliferated and formed premature tendon-like tissue using collagen type I sponge in vitro. There have been reports on MSCs culture on collagen type I sponge to treat tendon injury, all of which led to beneficial effects of constructs to improve healing. However, microstructural evaluation demonstrating progressive cellular proliferation and maturation in scaffold has not been reported. A report using similar methods to culture construct made of collagen type I sponge with ASCs cultured in tenogenic medium demonstrated progressive deposition of ECM onto scaffold without numerous cells embedded under histological evaluation. Numerous MSCs' colonization with parallel alignment along the direction of mechanical stimulation evident from histological evaluation of construct was reported previously with hydrogel-based formulation of neotendon. Similarly, numerous cell colonization within collagen-based scaffold was achieved by formulating cartilaginous construct made of human tendon-derived progenitor cells cultured on collagen type II sponge cultured in chondrogenic medium for 21 days. In that study, homogenously distributed numerous spindle-shape cells were observed in constructs. The discrepancy between this study and other previous studies may stem from simply the difference between properties of collagen type I and II. For example, it was shown that higher ratio of type I to type II collagen in hydrogel led to lower void space in comparison to 1:1 ratio, although higher percentage of type I increased superior mechanical properties. Another potential reason of numerous cell colonization observed in that study may be the additional cross-linking process of type II collagen sponge to reinforce mechanical properties. Collectively, the neotendon formulation protocol presents a novel method to construct highly cellular neotissue with organized structure. Additionally, findings on microstructural transformation over 21 days provides insight on detailed process of neotendon maturation.

The cellularity was low, and cells were spherical in constructs cultured in stromal medium when observed ultrastructurally from SEM images. However, collagen sponge contains both fibrous and sheet-like areas that mimic cellular morphology ultrastructurally. Therefore, confirmation of cellular attachment to collagen fibrils was not clearly demonstrated in stromal study. On the contrary, ultrastructure of constructs cultured in tenogenic medium in this study showed the presence of spindle-shape cells and aligning in parallel with surrounding cells especially after 21 days of culture. The spindle-shape of cells was like that of Scx-overexpressing human ESCs-derived MSCs (hESC-MSCs) on silk-collagen scaffold, although cells did not obtain parallel alignment in that study. In another study using silk-collagen scaffold with cultured hESC-MSCs, highly elongated cells with parallel alignment in the direction of dynamic mechanical stimulation along with highly upregulated Scx was achieved despite they were cultured in stromal medium, demonstrating dynamic strain is an effective method to promote tenogenesis.

Cyclic tensioning (dynamic strain) applied to constructs resulted in severely reduced cellularity. To date, it is widely accepted that cyclic tensioning improves viability of cells within scaffold and promotes tenogenic differentiation under tenogenic conditions, which in turn results in improved healing of injured tendon. The low cellularity of constructs cultured under cyclic tensioning may be due to lower attachment of cells to collagen fibrils evident from spherical morphology of cells observed both microstructurally and ultrastructurally. (FIGS. 14-15) Indeed, this cellularity lowering effects of strain was reported in the scientific literature even when strain was applied statically. For example, 20% static strain applied to collagen-based scaffold cultured with MSCs resulted in fewer cells compared to 0% strain, while 20% strain enhanced cell alignment in the direction of strain on scaffold with opposing gradients of mineral content and structural alignment. The spherical morphology of cells on collagen sponge network was similar to that of embryonic neural stem cells (NSCs) cultured on collagen-glycosamino glycan sponge ultrastructurally. Although the cell type and scaffold were different from those used herein, NSCs-collagen construct improved locomotion of mice affected by spinal cord injury. Therefore, it is possible despite of lower cellularity and weak attachment to scaffold, constructs cultured under dynamic tensioning may augment healing of tendon injuries. The duration of dynamic strain may be much shorter than that applied in our study. In a previous study, 2% strain for 30 minutes per day at 0.5 cycles per minute frequency was adequate to promote cellular proliferation and expression of tenogenic genes as well as ECM deposition in MSCs cultured on decellularized human umbilical vein using tenocytes extracts.

2. Healing Capacity of Implantable Collagen Constructs for Equine Tendon Regeneration in an Elongation-induced Rat calcaneal Tendinopathy Model

### 2.1. Introduction

Adult multipotent stromal cells (MSCs) have been directly injected into naturally occurring and experimentally induced tendon injuries with mixed and unpredictable results. The variability has been attributed to an engraftment efficiency of less than 0.001%. Additionally, injection of undifferentiated MSCs into tendon can have serious side effects including chondroid and ectopic bone formation at the injection site.

Neotissue generated by MSCs only or combined with COLI templates requires testing in an appropriate rodent tendinopathy model prior to preclinical equine testing. To date, the treatment effects of tendon neotissue has been most investigated in rodent acute tendon injury models created by surgical resection of portion of tendon. They were treated with MSCs alone, COLI scaffolds populated with MSCs, and tendon stem/progenitor cell (TSC) sheets, all of which resulted in improved mechanical strength and histological scores. However, caution needs to be taken with interpretation of these outcomes, since rodents possess stronger tendon regenerative potential and surgically created tendon defect models does not simulate clinical scenario characterized by chronic tendinopathy and subsequent rupture. Moreover, surgical implantation of neotissue at the time of injury is not a feasible approach for clinical application. More realistically, the optimum time to deliver exogenous cells to an injured tendon or ligament in horses is around the sixth day after injury at the transition between the inflammatory and subacute reparative phases.

In this regard, tendinopathy models that resemble clinical tendinopathy scenario both etiologically and pathologically, as well as administration strategies that are clinically feasible are essential to test efficacy and therapeutic value of novel treatment modalities. One of the most commonly used tendon for tendinopathy model creation is rat calcaneal tendon composed of insertions of the plantaris, gastrocnemius and soleus muscles due to its accessibility for controlled injury and subsequent treatment administration. The types of tendinopathy creation mechanisms range from chemical injury induction with collagenase, cytokines, prostaglandins and fluoroquinolone, or mechanical injury induction with electrical muscle stimulation and downhill/uphill running have been used across multiple animal species. Among these, downhill/uphill running can most closely recapitulate the overuse nature of tendinopathy which account for majority of naturally occurring tendon injuries in equine athletes. And rats underwent strenuous uphill treadmill running for 12 weeks were shown to have developed tendinopathy resembling naturally occurring human tendinopathy characterized by decreased collagen fiber organization, increased cellularity with endothelial cells and fibroblasts, while causing minimum inflammatory response.

However, strain injuries induced by these methods often can be inconsistent and subject to inherent individual variation. It is believed that mechanism of overuse tendinopathy is excessive loading and tensile strain beyond 4% of its length where the collagen fibers start to slide past one another as the intermolecular cross-links fail, and, at approximately 8% of elongation, a macroscopic rupture occurs because of tensile failure of the fibers and interfibrillar shear failure. Indeed, the elongation of superficial digital flexor tendon (SDFT) is a sign of tendinopathy presented as medial or lateral displacement, or sometimes as bowed tendon in horses. As such, surgical elongation of tendon using a tool devised to create strain injuries in canine ligaments is an appealing tendinopathy model creation modality, as it can consistently control strain level to minimize variations while simulating etiology of naturally-occurring tendinopathy.

Another important consideration in animal tendinopathy model creation is the role immune system plays in tendon healing. This is especially important, because varieties in expression level and haplotype of major histocompatibility complex (MHC) II within horse population had been reported to elicit immune responses despite of MSCs' general status as hypoimmunogenic to non-immunogenic from lack of MHC II expression. Moreover, haplotype mismatched allogenic MSCs are known to be short-lived in vivo upon transplantation, which contributes to failure of implants. Combining difficulties in identifying MHC II haplotype-matched donors among 50 known haplotypes and superior accessibility of allogenic versus autologous MSCs in horses, it is important to develop neotissue from allogenic MSCs that does not elicit immune response and augment healing in immunocompetent recipient individuals. Therefore, the use of both immunocompromised and immunocompetent rats to create tendinopathy model can elucidate the role immune system play in tendon healing by neotissue and leads to development of readily accessible immunocompatible neotissue.

In this study, tendon neotissue created from equine ASCs cultured on COLI template in tenogenic or stromal medium for 21 days under static strain, or phosphate buffered saline as no treatment were implanted via needle in randomly selected limb of each rat 6 days after bilateral elongation-induced calcaneal tendon injury creation. Hypotheses tested were: 1) tenogenic neotissue implanted is immunocompatible and regenerative without eliciting immune response and form tendon-like tissue, while stromal neotissue implant is immunocompatible yet non-regenerative without forming tendon-like tissue; and 2) the immune system plays a role in a mechanism of non-regenerative tissue formation by stromal neotissue. The objectives of the study were: 1) to compare microstructure of implanted both tenogenic and stromal neotissue in immunocompetent rat calcaneal tendon; and 2) to compare microstructure of implanted stromal neotissue in both immunocompetent and immunocompromised rat calcaneal tendon.

### 2.2. Materials and Methods

### 2.2.1. Study Design

Protocols were approved by the Louisiana State University Institutional Animal Care and Use committee prior to study initiation. calcaneal tendon elongation injury was made bilaterally to both hindlimbs of each Sprague Dawley (SD) rat (n=18) and RNU Nude (RNU) rat (n=12). Bovine corium COLI (Avitene™ Ultrafoam™ Collagen Sponge, Davol Inc., Warwick, RI) templates were infused with equine ASCs from 1 adult gelding and cultured in tenogenic (n=30) or stromal (n=15) medium for 21 days.

Six days after calcaneal tendon elongation injury, tenogenic neotissue (SD rat: n=18; RNU rat: n=12) was percutaneously injected into tendon lesion of one randomly selected hindlimb in each rat via needle. The opposite tendon lesion in each animal was injected with stromal neotissue (SD rat: n=9; RNU rat: n=5) or PBS (SD rat: n=9; RNU rat: n=5). Limb use was evaluated with an established rubric for all rats 1 week before surgery, daily up to 14 days after surgery and then weekly until harvest 6 weeks after surgery.

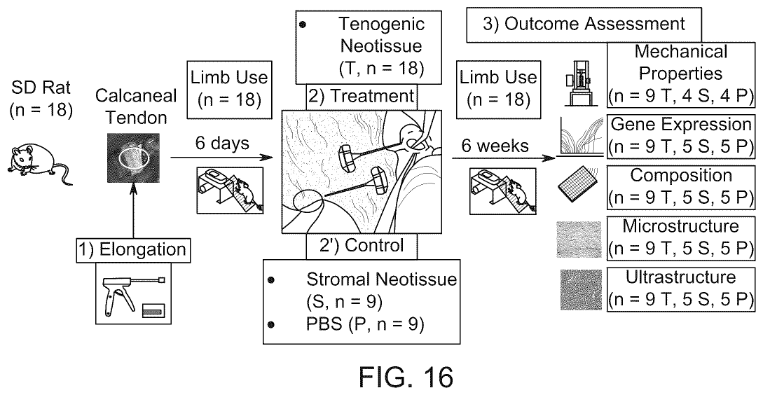

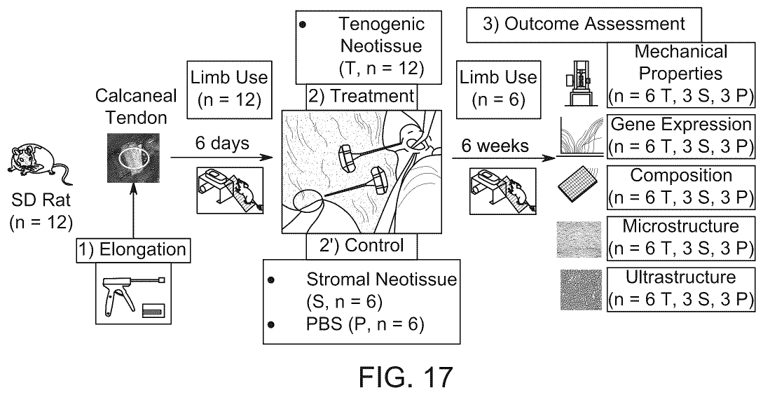

Tendons from 15 rats (SD rat: n=9; RNU rat: n=5) were evaluated for tensile mechanical properties. Tendons from the other 15 rats (SD rat: n=9; RNU rat: n=5) were divided sagittally to give four quadrants, medial axial, medial abaxial, lateral axial, and lateral abaxial. One quadrant from each rat was assigned to one of four outcome measures, gene expression, collagen composition, and micro- and ultrastructure (FIG. 16), based on a randomized block design.

### 2.2.2. Bioreactor System

COLI scaffold templates were secured to a movable horizontal bar under continuous uniaxial static tension and maintained inside a custom-designed bioreactor chamber that is connected to a perfusion system. The perfusion system consists of a 10 ml medium reservoir (Synthecon, Houston, TX) that permits gas exchange while a computer-controlled peristaltic pump (Ismatec 404b, Glattbrugg, Switzerland) maintains a bidirectional fluid flow rate of 1 ml/min. The cells were infused into COLI scaffold templates (6.0×4.0×1.0 cm³) at 1.0×106 cells/cm³ scaffold template through the injection port of each chamber and maintained in tenogenic (DMEM-high glucose, 1% fetal bovine serum (FBS), 10 ng/ml transforming growth factor (TGF)-β1, 50 mM L-ascorbic acid 2-phosphate sesquimagnesium salt hydrate, 0.5 mg/ml insulin, 1% antibiotics) or stromal (DMEM-F12, 10% FBS, 1% antibiotics) medium. The perfusion system was maintained in 5% CO2 at 37° C. for 21 days with medium changes every 7 days. (FIGS. 3-4)

### 2.2.3. Elongated Calcaneal Tendinopathy Models

Both Sprague Dawley (SD) and RNU nude (RNU) rats aging 8-10 weeks old and weighing 200-250 g were obtained from a vendor (Charles River Laboratories, Wilmington, MA). Elongation-induced calcaneal tendinopathy was created in both hindlimbs of rats using an adaptation of an elongation device validated for canine cranial cruciate ligament elongation.

Briefly, rats were induced and maintained with isofluorane (2.0%) in 100% oxygen at a flow rate of 1 L/min inside an induction chamber and then a facemask. Premedication was given by subcutaneous injection of glycopyrrolate (0.02 mg/kg) and butorphanol (0.5 mg/kg). Following aseptic preparation, L-shaped skin incisions were made superior and lateral to each calcancal tendon to elevate the skin. After carefully excising the paratenon of each calcancal tendon, fine grit stainless steel nail file was placed underneath the tendon to prevent slippage during force application.

Single-interrupted #4-0 Vicryl® sutures (Ethicon, Somerville, NJ) were placed through the calcaneal tendon with the distal suture just proximal to the calcaneus and the proximal suture at the junction of the calcaneal tendon and gastrocnemius muscle. A spring scale was used to apply 2.5 N of tension to the proximal aspect of the calcaneal tendon via stainless steel S-shape hook and #0 PDS® II suture (Ethicon). The distance between the distal edge of the proximal suture and the proximal edge of the distal suture was measured during force application to determine the pre-elongation length (PrE) with a vernier caliper.

The tendon elongation device has a hollow cylinder (7.5 mm inner diameter; 10 mm outer diameter) with a 2.5 mm-wide textured (80 grit) rim. Within the cylinder, a removable 10-gauge stainless steel hook (5 mm diameter, 2 mm depth) is attached to a retractable ratchet system that locks every 1.5 mm. The hook was placed beneath calcaneal tendon midway between sutures. The textured surface of the cylinder engaged with the tendon surface by pressing the device firmly to prevent slippage during hook retraction and limit elongated tissue to the area within the inner diameter of cylinder. The hook was retracted 1 ratchet step (1.5 mm) to elongate the isolated section by 8 ratchet steps (12 mm) and held for 1 minute until release.

Post-elongation length (PoE) was determined identically to the PrE, and percent elongation (PE) was calculated as PE=(PoE−PrE)/PrE×100. Skin was approximated with #4-0 Vicryl® sutures in a simple continuous subcuticular suture pattern. The suture line was be sealed with tissue adhesive. Post-medication was given by subcutaneous injection of carprofen (5 mg/kg), enrofloxacin (10 mg/kg), and 5 ml of warmed 0.9.

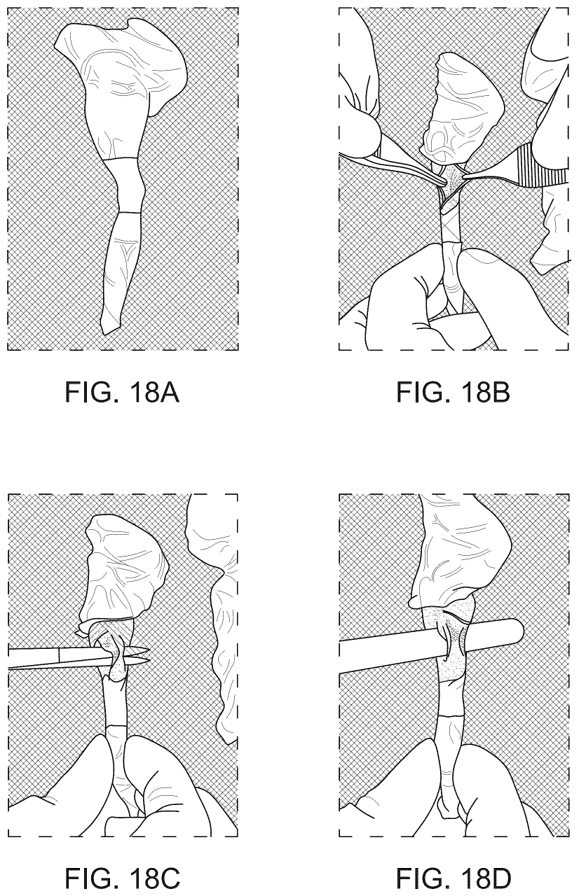

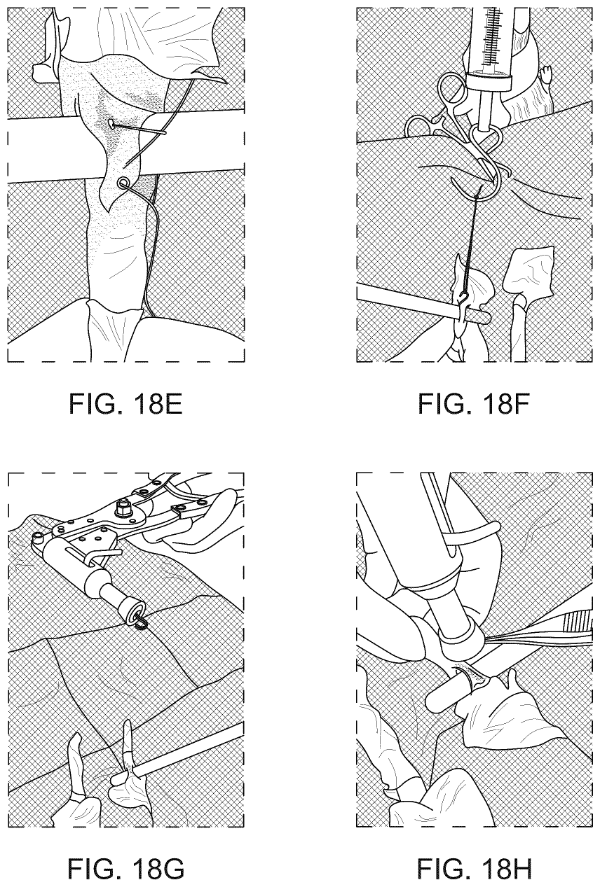

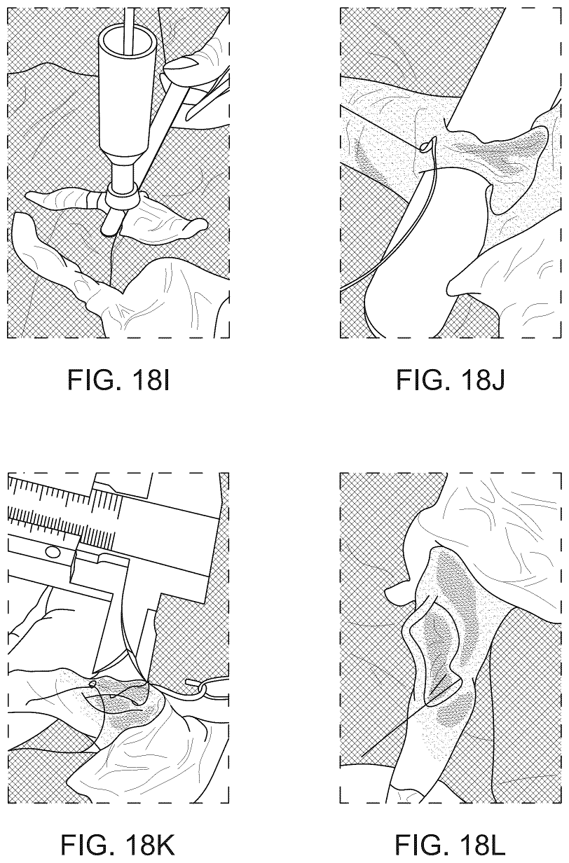

Elongation-induced calcaneal tendinopathy model creation. (FIGS. 18A-I) Hindlimb was passed through fenestrated drape and both proximal and distal limb covered with iodine-impregnated incision drape (FIG. 18A), L-shaped skin incisions was made superior and lateral to each calcancal tendon to elevate the skin (FIG. 18B), isolate and elevate calcancal tendon (FIG. 18C), flat stainless steel stick with etched surface was placed underneath tendon (FIG. 18D), single-interrupted 5-0 Vicryl® sutures were placed through the calcancal tendon with the distal suture just proximal to the calcaneus and the proximal suture at the junction of the calcaneal tendon and gastrocnemius muscle (FIG. 18E), a bar type tension gauge applied 2.5 N of tension to the calcancal tendon (FIG. 18F), tendon elongation device (FIG. 18G), lift tendon with the hook inside at midway between proximal and distal sutures (FIG. 18H), textured surface of the cylinder was engaged with the tendon surface by pressing the device firmly to prevent slippage during hook retraction and limit elongated tissue to the area within the inner diameter of cylinder (FIG. 18I) and retract tendon inside cylinder. The distance between proximal and distal suture of elongated tendon (FIG. 18J) was measured with a vernier caliper. Subcutaneous tissue and skin were closed in routine manner (FIG. 18L)

### 2.2.4. COLI Construct Implantation

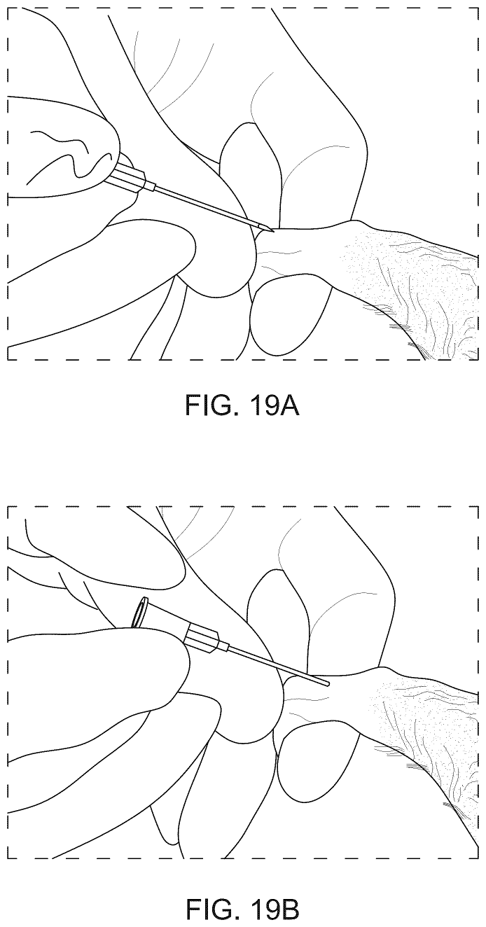

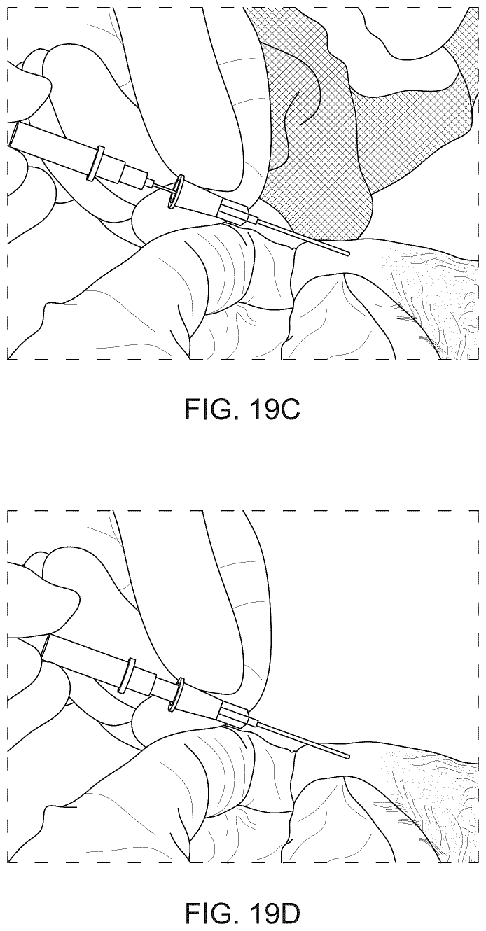

Six days after tendon elongation surgeries, ASC-COLI constructs cultured in tenogenic or stromal medium, or PBS were implanted via needle under general anesthesia identically as for surgery. A half of cylindrical piece (4 mm diameter, 10 mm thickness) collected from construct using biopsy punch was placed into the tip of a 18-gauge 1.5 inch needle (Exel international, Salaberry-de-Valleyfield, Canada), 20-gauge 1 inch needle (Becton Dickinson, Franklin Lakes, NJ) inserted into 18-gauge needle from the tip side, then construct pushed into 20 gauge needle using stylet of 22-gauge 1 inch IV catheter (Terumo Medical Corporation, Southaven, MS). After aseptic preparation of hindlimb, 20-gauge needle was inserted transdermally into calcaneal tendon to proximal end of tendon, and construct was implanted by advancing 22-gauge IV catheter stylet inside 20-gauge needle while withdrawing needle. (FIGS. 19A-D) Needle implantation of ASC COLI construct into rat calcancal tendon. A half of cylindrical piece (4 mm diameter, 10 mm thickness) placed into the tip of 20-gauge 1 inch needle was inserted into calcaneal tendon transdermally from distal (A) to proximal (B) end of tendon. Stylet of 22-gauge 1 inch IV catheter was inserted into 20-gauge needle (C) and advanced while withdrawing needle to implant construct.

The procedure was performed on both limbs, with one randomly assigned to receive a tenogenic ASC-COLI construct, while the other received a stromal ASC-COLI construct or PBS (0.5 ml). Rats were maintained in cages without movement restriction for 6 weeks after which they were humanely sacrificed according to current AVMA standards and calcancal tendons harvested.

### 2.2.5. Functional Outcome

All rats were monitored once daily for signs of pain according to the Rat Grimace Scale (RGS) 103,104, and for excessive swelling, redness or drainage at the surgery site. A scoring system previously developed to evaluate rat limb use 105 was used to evaluate limb use one week before surgery, daily up to 14 days after surgery, and then weekly up to 6 weeks.

### 2.2.6. Microstructure

A quadrant of calcaneal tendon was fixed in 4% paraformaldehyde (PFA), paraffin embedded, sectioned (5 μm) and stained with haematoxylin and eosin or Masson's trichrome. Digital images generated with a slide scanner (NanoZoomer, Hamamatsu Photonics K.K, Hamamatsu City, Japan) or a light microscope (DM4500B, Leica, Wetzlar, Germany) fitted with a digital camera (DFC480, Leica) and cellular morphology, distribution, and extra cellular matrix organization were be evaluated.

### 2.2.7. Gene Expression-RT-PCR

A quadrant of calcaneal tendon was snap frozen in liquid nitrogen and transferred to a cooled grinding cylinder of BioPulverizer (BioSpec, Bartlesville, OK) and pulverized. 106 Ground samples were collected by rinsing the cylinder with 1 ml of TRI Reagent® (Sigma, St. Louis, MO) and transferred to 1.5 ml tube. Ground sample was homogenized by passing mixture through 18-gauge needle 30 times. Homogenate was spun down at 21,000×g for 15 minutes at 4° C. Total RNA was extracted from supernatant by phenol-chloroform extraction according to the manufacturer's instructions. Isolated RNA was cleaned up by RNeasy® Mini Kit (QIAGEN, Hilden, Germany). One microgram of total RNA was used for cDNA synthesis (QuantiTect® Reverse Transcription Kit, QIAGEN). Rat-specific primers for tendon-specific genes, collagen 1a1 (Col1a1), collagen 3a1 (Col3a1), tenascin-C (TnC), scleraxis (Scx), and tenomodulin (Tnmd) were quantified using primers previously validated.39 PCR was performed with denaturation step at 95° C. for 15 minutes, followed by 40 cycles of denaturation at 94° C. for 15 seconds, annealing at 52° C. for 30 seconds, and elongation at 72° C. for 30 seconds using SYBR Green system (QuantiTect® SYBR® Green PCR Kits, QIAGEN). Relative gene fold change was determined by standard means (2-ΔΔCt). Glyceraldehyde 3-phosphate dehydrogenase (GAPDH) was used as the reference gene.

TABLE 2
Rat-specific Primer Sequences

| | | | SEQ | |
|---|---|---|---|---|
| | | | ID | Accession |
| Gene | | Sequence (5′ - 3′) | NO: | Number |
| Col1a1 | Fwd | GGAGAGAGTGCCAACTCCAG | 31 | NM_053304.1 |
| | Rev | GTGCTTTGGAAAATGGTGCT | 32 | |
| Col3a1 | Fwd | TCCCAGAACATTACATACCACT | 33 | NM_032085.1 |
| | Rev | GCTATTTCCTTCAGCCTTGA | 34 | |
| TnC | Fwd | AGATGCTACTCCAGACGGTTTC | 35 | NM_053861.1 |
| | Rev | CACGGCTTATTCCATAGAGTTCA | 36 | |
| Scx | Fwd | AACACGGCCTTCACTGCGCTG | 37 | NM_001130508.1 |
| | Rev | CAGTAGCACGTTGCCCAGGTG | 38 | |
| Tnmd | Fwd | GTGGTCCCACAAGTGAAGGT | 39 | NM_022290.1 |
| | Rev | GTCTTCCTCGCTTGCTTGTC | 40 | |
| GAPDH | Fwd | AAGTTCAACGGCACAGTCAAGG | 41 | NM_017008.4 |
| | Rev | CGCCAGTAGACTCCACGACATA | 42 | |

### 2.2.8. Statistical Analysis

Results are presented as mean±standard error of the mean (SEM). Normality of data was examined with the Kolmogorov-Smirnov test. Measures of functional outcome, post-elongation percentages, and post-elongation tendon width were compared with ANOVA among groups. When overall difference was detected, pairwise comparisons between groups were performed using Tukey's post-hoc test. Fold changes of tenogenic gene expression were compared: to 1 using one sample t-test and between groups using two-sample t-test for normally distributed results; and to 1 using Wilcoxon signed rank test and between groups using Mann Whitney test for non-normally distributed results. All analyses were conducted using Prism (GraphPad Software Inc., San Diego, CA) with significance considered at p<0.05.

### 2.3. Results

### 2.3.1. Functional Outcome

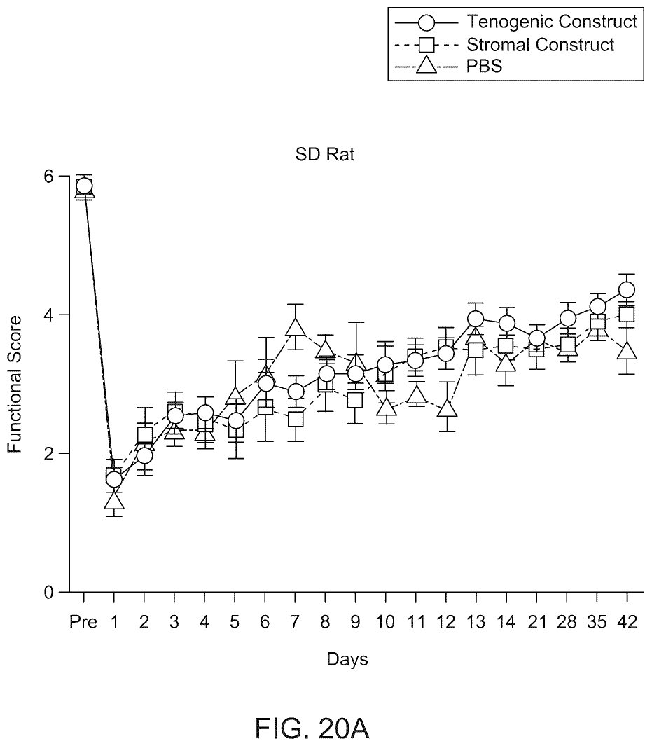

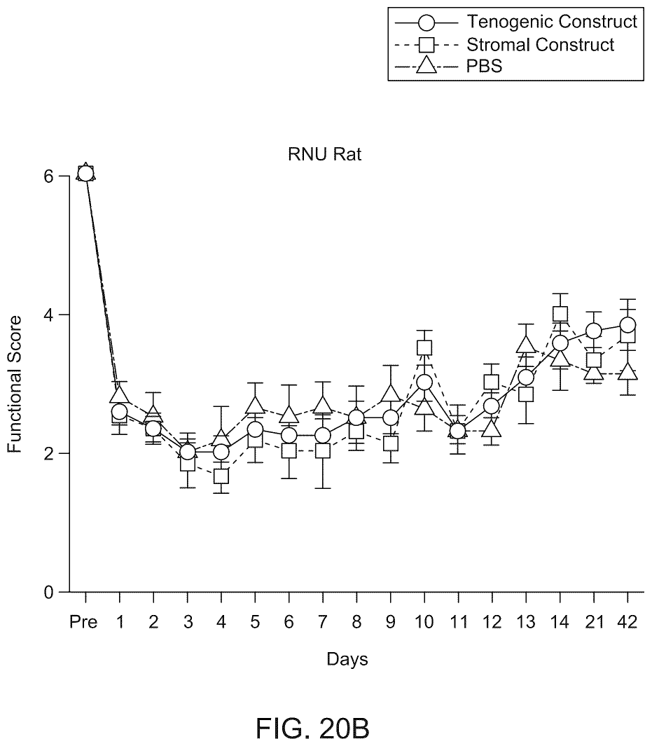

Following elongation injuries, functional scores dramatically decreased despite rats were ambulatory all the time. Additionally, minimum swelling was noted at the surgery site without signs of infections or excessive inflammation for all rats. Functional scores gradually recovered over 6-week period for both SD and RNU rats (FIGS. 20A-B, respectively). However, SD rats appeared to have better recovery of limb use than RNU rats by the end of post-operative period. After 6 weeks of post-operative period, neither SD rats nor RNU rats fully regained original limb function.

Compared to SD rats, functional recovery of limbs in RNU rats appeared to be biphasic rather than continuous. Functional score of hindlimbs were measured in SD (FIG. 20A) and RNU (FIG. 20B) rats from pre-injury to 6 weeks post-injury. Functional scores were obtained for limbs that received tenogenic constructs (circle), stromal constructs (square), or PBS (triangle). Each treatment was administered at day 6. In SD rats, functional scores had a trend of low values in PBS treatment group compared to tenogenic construct or stromal construct groups after 10 days post-operatively. There appeared to be no difference in functional scores between tenogenic construct and stromal construct groups (FIG. 20A). On the contrary, there was no trend of difference in functional scores among treatment groups for RNU rats all the time (FIG. 20B).

### 2.3.1. Post-Elongation Percentage and Tendon Width

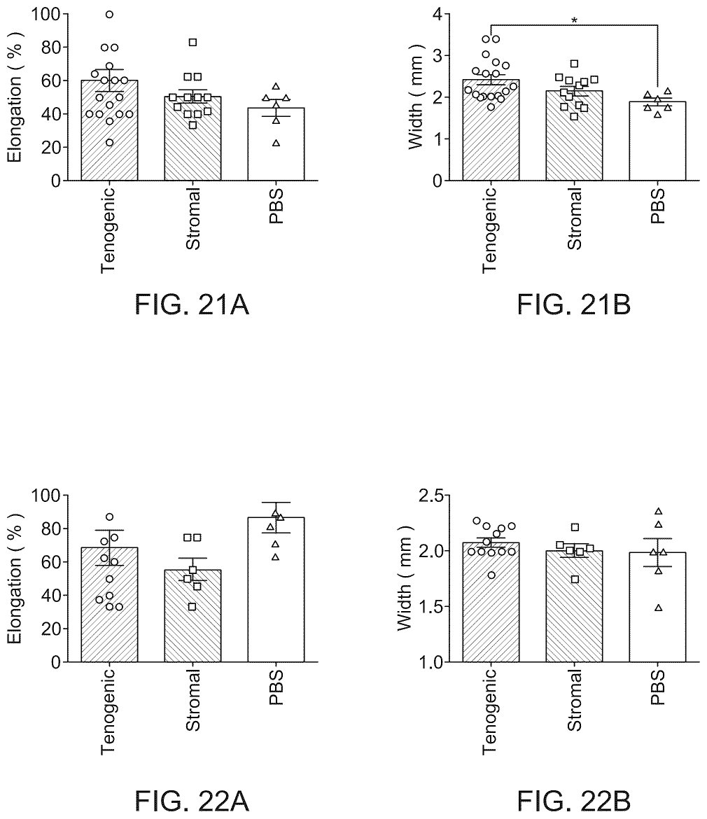

Percentage of elongation did not differ among treatment groups for SD rats (FIG. 21A). Overall elongation percentage was 54.24±3.756%. Elongation percentages were generally higher than those that may lead to acute rupture. As a result, loosened midportion of calcancal tendon following elongation between sutures was noted (FIG. 18J). Individually, elongation percentages were 60.12±6.666% for tenogenic construct group, 50.65±3.889% for stromal construct group, and 43.79±4.984% for PBS group. Variation in elongation percentages was higher in tenogenic construct group, and as high as 144% of elongation was achieved without causing complete rupture.

Six weeks post-injury, the widths of healed calcaneal tendon at midportion was higher in tenogenic construct group (2.444±0.1175 mm) compared to PBS group (1.903±0.08724 mm). Widths of calcaneal tendon in stromal construct group (2.164±0.1088 mm) were not different from tenogenic construct group or PBS group (FIG. 21B). There was no visible partial or complete rupture noted in gross appearance of healed tendons among all treatment groups. Post elongation percentage was determined at the time of surgery and width measured at tissue harvest for each tendon of all rats.

Similar to SD rats, percentage of elongation did not differ among treatment groups for RNU rats (FIG. 22A). Overall elongation percentage was 70.17±6.297% and appeared to be higher than that for SD rats. Individually, elongation percentages were 68.91±10.64% for tenogenic construct group, 55.72±6.789% for stromal construct group, and 87.16±9.224% for PBS group. Variation in elongation percentages was again higher in tenogenic construct group, and as high as 150% of elongation was achieved without causing complete rupture. High variability demonstrated less control in size of injury compared to transection of tendon.

Unlike SD rats, the widths of healed calcancal tendon at midportion in RNU rats 6 weeks after injury were not different among treatment groups. Individually, widths were 2.082±0.04139 mm for tenogenic construct group, 2.012±0.06183 mm for stromal construct group, and 1.992±0.1262 mm for PBS group. Although non-significant, there was a trend of widest tendon widths by tenogenic construct treatment, followed by stromal construct and PBS treatment. There was also no visible partial or complete rupture noted in gross appearance of healed tendons among all treatment groups in RNU rats.

### 2.3.2. Microstructure

In calcaneal tendons of SD rats who received tenogenic constructs, there were no obvious regions of distorted cellular alignment or ECM orientation at the midportion where elongation injury was created. Implanted tenogenic constructs were clearly identified as cylinder-like structure with basophilic in H & E staining and light blue in trichrome staining within implanted calcaneal tendon of rats at the time of harvest (FIGS. 23A-F). Although implants were ejected from needle while withdrawing from calcaneal tendon along the entire length, majority of portion of implants appeared to have been deposited at the proximal end of tendons (FIG. 23A). However, lesser amounts of implants were deposited along the entire length of tendons. Implants were relatively acellular and mostly consisted of fibrous ECM and diffusely residing fibroblastic cells. Cellularity within implants were relatively higher than surrounding native tendon, yet low cellularity of implants resembled native tendon tissue. In terms of biocompatibility, there were no signs of mononuclear cells' infiltration, indicating lack of immune response to implants. Of note was the presence of red blood cells within empty spaces of implants, which suggested neovascularization was also induced (FIG. 23F).

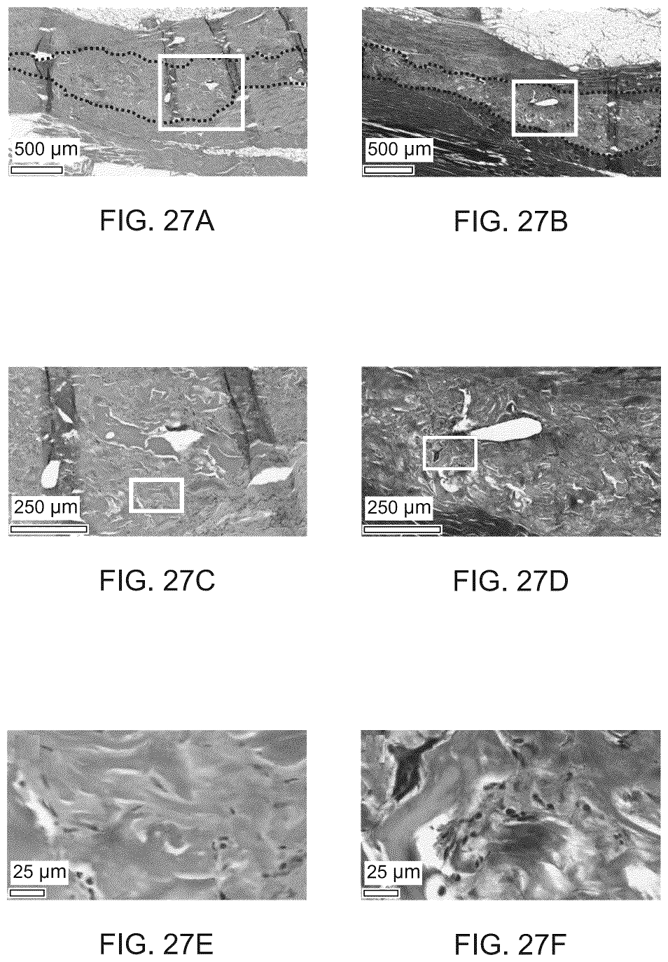

Majority of cells assumed tenocyte-like morphology with elongated rhomboid nucleus. There were areas where cells aligned randomly inside the implant, while there were areas where cells obtained parallel alignment with surrounding native tendon (FIG. 27D). In the areas where cells aligned randomly, cells tended to form clusters with relatively higher density. Around those areas, fibrous ECM also maintained random alignment. In the areas where cells aligned parallelly, cellularity was lower and cells aligned parallel to the surrounding tendon tissue. Additionally in those areas, fibrous ECM within gained parallel alignment as well to the surrounding native tendon. Additionally, these ECM developed crimp pattern characteristic of tendon tissue (FIG. 23D).

The immediate adjacent areas of implants did not accumulate fibrotic tissue to sequester implant. This again indicated lack of foreign body reaction to implant. Moreover, there were apparent integration of implants with surrounding native tendons characterized by gradual transition of implants towards native tendons merging ECMs from both areas (FIG. 23E). Cells within merging areas also appeared to be more elongated mature tenocyte-like cells.

Microstructures of calcancal tendon treated with tenogenic construct in SD rats are shown in FIGS. 23A-F. Harvested calcaneal tendon was stained with hematoxylin and eosin (FIGS. 23A, C, E) or Masson's trichrome (FIGS. 23B, D, F). Implanted construct was indicated with black dotted line. Panels E, F, C, and D, are enlarged images represented by rectangles in panels C, D, A, and B, respectively. Scale bars=1 mm (A, B), 100 μm (C, D), and 25 μm (E, F).

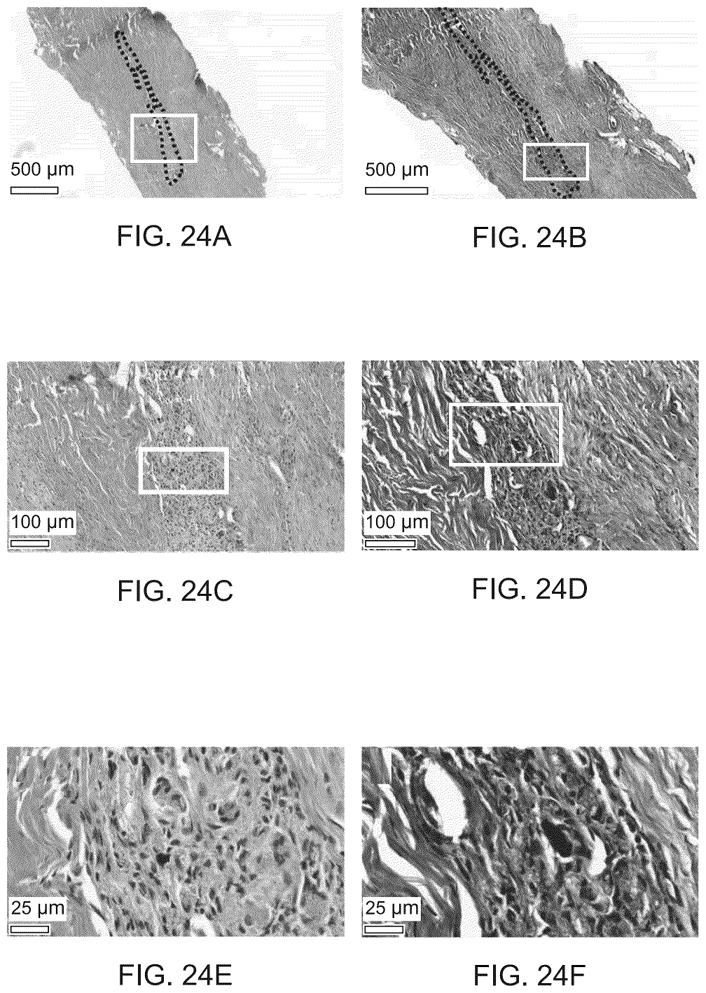

Like tendons that received tenogenic construct administration, those with stromal constructs also lacked obvious disorganized area in the midportion. The implants were also clearly identified as cylindrical structure within tendon demarcated from surrounding tissue. The implants did not have infiltrating monocytes or surrounding fibrous encapsulation. Cells within implants appeared to be more round to rhomboid with oval nucleus. Implants were high in cellularity and contained less amounts of immature ECM that was stained red by trichrome (FIGS. 24A-F). Microstructure of calcancal tendon treated with stromal construct in SD rats. Harvested calcaneal tendon was stained with hematoxylin and eosin (A, C, E) or Masson's trichrome (B, D, F). Implanted construct was indicated with black dotted line. Panels E, F, C, and D are enlarged images represented by rectangles in panels C, D, A, and B, respectively. Scale bars=500 μm (A, B), 100 μm (C, D), and 25 μm (E, F).

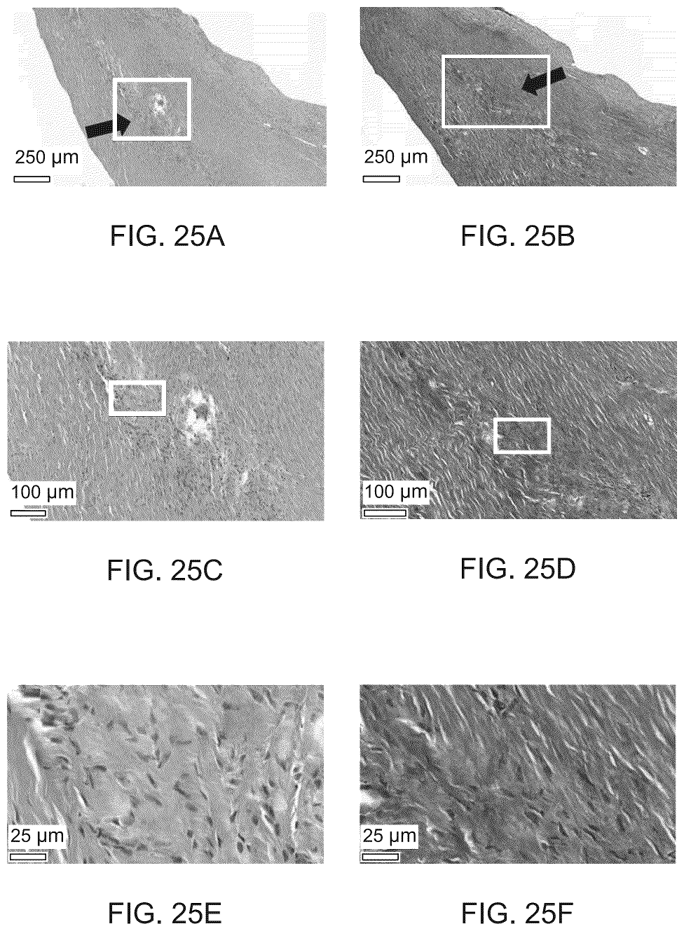

Tendons that received PBS treatment had apparent disorganized areas at the midportion characterized by high cellularity and non-parallel alignment of both cells and ECM. However, majority of areas within tendon maintained low cellularity with parallel alignment along with longitudinal direction (FIG. 25). There were no signs of hematoma formation at the time of harvest. Additionally, abnormal cellular differentiation such as chondrocyte-like or osteocyte-like cell formation was not evident as well. Microstructure of calcaneal tendon treated with PBS in SD rats. Harvested calcancal tendon was stained with hematoxylin and eosin (A, C, E) or Masson's trichrome (B, D, F). Transverse lesion in the mid-substance of tendon was indicated with black arrow. Panels E, F, C, and D, are enlarged images represented by rectangles in panels C, D, A, and B, respectively. Scale bars=250 μm (A, B), 100 μm (C, D), and 25 μm (E, F).

Similar to tendons of SD rats that received implant treatments, the tendons of RNU rats which received tenogenic constructs did not show obvious elongation-induced lesions. Implants in the tendons were clearly identified as cylindrical structures (FIG. 26). They were basophilic in H & E staining and light blue in trichrome staining. The areas stained red by trichrome staining were observed in multiple portions, and it was not consistent finding with tenogenic constructs implanted into SD rats' tendons. The organization of cells and ECM appeared to be less organized than tenogenic constructs implanted in calcaneal tendons of SD rats.

Although cellularity was low homogenously, much less cells aligned parallelly to each other and to the surrounding native tendons (FIG. 26D). Distinct forms of ECM were observed within implant, which were fibrous and caseous appearance. Among these, fibrous ECM obtained parallel to oblique alignment with longitudinal direction of native tendon (FIG. 26F). Of note was stronger blue coloration of fibrous ECM than tenogenic constructs implanted tendons of SD rats by trichrome staining. The degree of staining with blue color was almost indistinguishable between fibrous ECM within implants and collagen fibers of native tendons, potentially more advanced maturity of fibrous ECM. The same areas with fibrous ECM also had close eosinophilic coloration to native collagen fibers. Cells had oval to spindle shape nucleus and populated more frequently in the areas where fibrous ECM existed. Cellular nucleus was also intact, and no cells had apoptotic or necrotic appearances. Cells were also not assuming chondrocyte-like morphology characterized by round cells embedded in hyaline-like ECM, or osteocyte-like morphology characterized by calcium deposition around them. Also, there was no cells that contain vacuoles resembling adipocyte-like cells or macrophages.

As expected from immunocompromised status of RNU rats, there was no sign of immune reactions characterized by monocyte infiltration or encapsulation of implants by fibrous tissues (FIG. 26C). Panels E, F, C, and D, are enlarged images represented by rectangles in panels C, D, A, and B, respectively. Scale bars=1 mm (A, B), 250 μm (C, D), and 25 μm (E, F). Not only within implants themselves, but also within native tendons around implants lacked infiltration of inflammatory cells. Unlike tenogenic constructs implanted into calcaneal tendons of SD rats, presence of red blood cells within implants was not noted, indicating lack of neovascularization. In terms of integration with surrounding native tendon structure, the boundary between implants and native tendons was clear and lacked insertion of collagenous fibers from native tendons to implants, suggesting limited integration of implants (FIGS. 26C-D). Microstructure of calcaneal tendon treated with tenogenic construct in RNU rats. Harvested calcancal tendon was stained with hematoxylin and eosin (A, C, E) or Masson's trichrome (B, D, F). Implanted construct was indicated with black dotted line.

Clear elongation lesions were not observed in tendons of RNU rats that received stromal constructs. Implants appeared similar to those of tenogenic constructs (FIGS. 27A-F). Panels E, F, C, and D, are enlarged images represented by rectangles in panels C, D, A, and B, respectively. Scale bars=500 μm (A, B), 250 μm (C, D), and 25 μm (E, F). Cells were mostly maintaining viable morphology with intact nucleus. Like tenogenic constructs, both fibrous and caceous ECM existed with more cellularity within areas of fibrous ECM (FIG. 27C). The organization of fibrous ECM was random, and lesser blue coloration with trichrome staining suggested less mature fibers (FIG. 27F). Immune response was not observed. Microstructure of calcancal tendon treated with stromal construct in RNU rats. Harvested calcaneal tendon was stained with hematoxylin and eosin (A, C, E) or Masson's trichrome (B, D, F). Implanted construct was indicated with black dotted line.

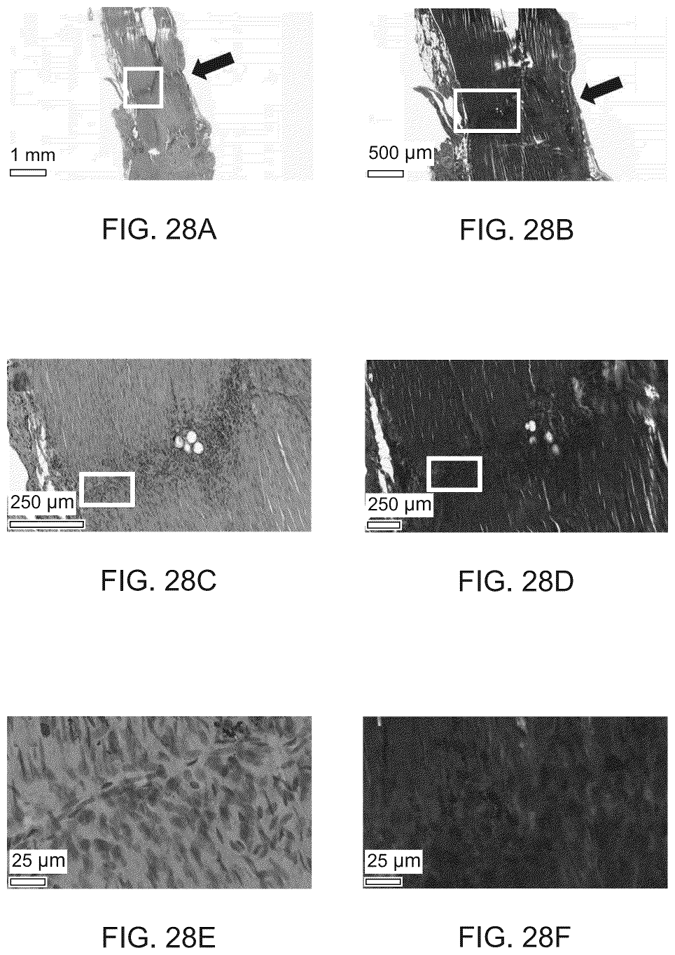

In the tendons of RNU rats that received PBS treatment, elongation-induced lesion characterized by higher cellularity and disorganized cell-ECM orientation was observed (FIGS. 28A-B, black arrows). Panels C, D, E, and F are enlarged images represented by rectangles in panels A, B, C, and D, respectively. No areas contained apoptotic- or necrotic-like cells, and had excessive inflammatory changes, indicating the tendons were in the phases of both proliferative and remodeling (FIGS. 28E-F). Some areas had apparent chondroid formation with circular non-stained proteinaceous deposition that contain round cells (FIGS. 28C-D).

### 2.3.3. Gene Expression RT-PCR

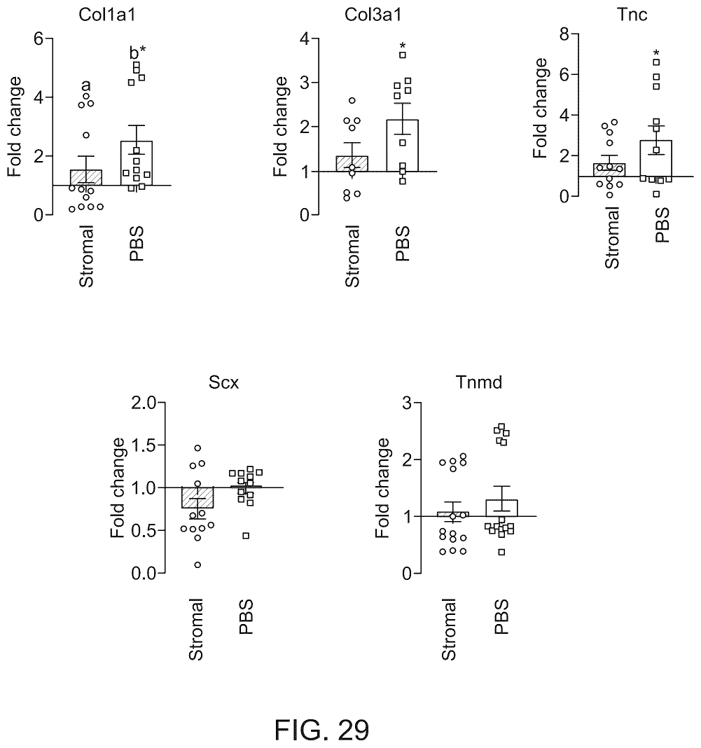

There was a trend of higher gene expression in tenogenic construct treated tendons over both stromal construct or PBS treated tendons of SD rats (FIG. 29). However, the difference was larger with PBS treated tendons. Moreover, tenogenic construct treated tendons had higher expression than PBS treated tendons significantly only for Col1a1, Col3a1, and TnC. Tendon-specific gene expression of SD rat calcaneal tendons treated by tenogenic constructs, stromal constructs (Stromal), or PBS 6 weeks post-injury. Gene expressions of calcaneal tendons treated with tenogenic constructs were normalized to those treated with stromal constructs (orange) or PBS (white) and expressed as fold change. Data presented as mean±SEM. Asterisks indicate difference from 1-fold within each stromal construct and PBS group (difference from tenogenic construct group).

### 2.4. Discussion

The injury created in the study by elongation device was unique in that it recapitulated acute overstrain on calcaneal tendon. Acute calcaneal tendon rupture is the most common tendon rupture in the lower extremity and occur typically in individuals who are active only intermittently, a different etiology from overuse calcaneal rupture. Therefore, although calcaneal tendon rupture was not created, an elongation-induced injury model was likely to represent the most common type of calcaneal tendon injury in human. A similar strain-induced tendon injury model was reported previously using cyclic strain of rat patellar tendon to attain fatigue damage accumulation. The limb use of rats after elongation injury was impaired immediately, yet gradually recovered in both immunocompetent and immunocompromised rats. And rats were ambulatory throughout the study period. However, limb use evaluated in our study using subjective scoring showed no difference among treatments regardless of rat phenotypes. A study evaluating limb use after collagenase-induced patellar tendon injury in rats observed rather subtle impairment of gait parameters by injuries, and the parameter that was associated with pain was double stance duration. Moreover, it was revealed a simple needle injection of saline into tendon severely impaired many of gait parameters evaluated. Therefore, it was possible, not only elongation-induced injury but also needle implantation of constructs in our study led to pain response in rats and indistinct recovery among treatments. Additionally, step length and ground reaction forces were the only affected parameters after RC tear and repair in rats, which was also not different from uninjured limb by day indicating objective gait analysis system is necessary to detect changes in gait after tendon injury and treatment.

Although not statistically different, the recovery of limb used in immunocompromised rats appeared to be slower than that of immunocompetent rats in the present study. Athymic RNU rats used were depleted of T cells, and it was reported M1/M2 macrophage ratio peaked at day 3, while T helper and Treg cells increased over time during healing of transected calcancal tendon.¹¹¹ The importance of T cells in tendon healing was also demonstrated in the study reporting expression of chemokine receptor 2 (CCR2), an important surface marker normally expressed in circulating bone-marrow monocytes and a mediator of macrophage recruitment, in both macrophage and T cells of healthy tendon. And knockout of CCR2 resulted in reduced numbers of myofibroblasts and reduced functional recovery during late healing.

Regarding tendon width, it is commonly seen the size of healing tendon often assessed by cross-sectional area (CSA) or thickness decrease with during healing. And injection of ASCs was reported to decrease thickness of recalcitrant patellar tendinopathy along with decreased pain scores in 6 months period. In the present study, the width of the healed calcancal tendons treated by tenogenic constructs were larger than those treated by PBS. This was consistent with the increased CSA coincided with increased maximum load and stiffness of rat calcaneal tendon both after 2 and 4 weeks of mid-substance defect creation followed by tendon stem/progenitor cell sheet wrapping compared to non-treatment. The difference between those studies on the effects of cellular therapies may largely stem from differences in responses of tendons to injuries. Indeed, ratio of CSA of ruptured calcaneal tendons to that of contralateral healthy tendons increased gradually upon primary surgical repair up to 6 months and decreased by 12 months in human. Therefore, increase of healed tendon width at earlier stages and decrease at later stages both may represent a better healing.

Regarding the percentage of elongation after strain in the present study, average 50-70% elongation of calcaneal tendon immediately after strain application was achieved for both immunocompetent and immunocompromised animals. It is an extensive elongation which may result in acute rupture based on established stress-strain curve of tendons, since macroscopic rupture starts at above 8% elongation. Although elongation achieved in the present study is not directly comparable to post-injury calcancal tendon elongation commonly observed in human after acute rupture, our model may recapitulate a clinical scenario of strain-induced acute calcancal tendon rupture in human. In human, calcaneal tendons were reported to elongate 0.15 to 3.1 cm after acute injury and healing, approximately 0.8-16% elongation of original length, given average length of calcancal tendon is approximately 18 cm. In a previous study, the failure strain of human calcaneal tendon was on average 12.8% for the bone-tendon complex and 7.5% for the tendon substance at the 1% per second rate strain application, whereas mean failure strain was 16.1% for the bone-tendon complex and 9.9% for the tendon substance at 10% rate. In rats, strain at failure of transected and healed calcaneal was reported to be approximately 1.0 cm, which is close to 70% elongation for non-repaired calcaneal tendon. It was reported primary repair of transected calcaneal tendon maintain original length of approximately 1.0 cm post-operatively, whereas non-repaired tendon resulted in callus formation and approximately 60% increase of original length to 1.6 cm after 8 weeks. In uninjured calcancal tendon of rat, 2 bundles that consist calcaneal tendon, plantaris longus and gastrocnemius tendons, both reached approximately 20% strain at failure. Collectively, evidence suggests rat calcaneal tendon tends to withstand higher strain without rupture than human, and it may have led to high elongation percentage achieved in the present study. This discrepancy may be attributed to extensive rotation of 2 calcaneal tendon bundles in rat, which potentially allows more elongation under stress.

The appearance of constructs cultured in stromal medium were more disorganized with randomly oriented round to oval cells surrounded by much fewer collagenous ECMs stained blue with trichrome staining after implanted into immunocompetent rats. This contradicted the similar composition of constructs cultured in both stromal and tenogenic media after implanted into immunocompromised rats. This discrepancy revealed the roles that immunology plays to maintain phenotype of implanted constructs and direct maturation of implanted neotissue towards orthotopic tissue. Interestingly, it was reported that undifferentiated xenogeneic human MSCs had better retention in subcutaneous space of immunocompetent mice with less lymphocyte and macrophage infiltration than osteogenically differentiated MSCs. Similarly, allogenic MSCs differentiated to myocytes increased their immunogenicity by upregulating major histocompatibility complex (MHC)-Ia and -II, resulting in earlier elimination from infarcted heart of rats and lost functional improvement by 5 months. Therefore, pre-implantation differentiation may elicit a stronger immune response to both xenogencic and allogenic MSCs, which contrasted with our findings. Previously, MSCs delivered in collagen hydrogel implanted into the central defects of rabbit patellar tendons resulting in ectopic bone formation up to 28% of treated tendons. This indicates that undifferentiated MSCs do not tend to maintain undifferentiated state upon implantation and may differentiate into ectopic lineages. The present results indicate mechanisms of this ectopic tissue formation of MSCs are partly immune-related. The importance of immune status as well as scaffold types were previously demonstrated clearly in a study investigating sarcoma formation by implanted MSCs. In that study, host-derived sarcomas developed, when MSC/collagen sponge constructs were implanted subcutaneously into syngeneic and immunocompromised mice, but not when allogeneic MSCs were used or MSCs were injected as cell suspensions.

In studies, calcaneal tendons had mild signs of tendinopathy such as increased cellularity or loss of parallel cell alignment along longitudinal axis regardless of treatment in both immunocompetent and immunocompromised rats and lacked signs of severe tendinopathy such as calcified deposits or chondroid formation that can be formed by collagenase injection. Additionally, the lesion was not limited to the mid-substance and found inconsistently throughout the length of the tendons. This may be attributed to the large size of hook used to lift tendons and hollow cylinder that was used to prevent slippage of tendon while applying strain. As a result, the stress might have concentrated at both proximal and distal contacts between the cylinder and tendon. Moreover, robust healing capacity of rat calcaneal tendon was evident from the study reporting reattachment and histological characteristics of only increased cellularity and lost parallel fiber orientation as early as 21 days after transection and saline treatment. Robust healing of tendon was further prominent especially in young rats compared to old rats. For example, 20 months old SD rats showed lower histological scores and more adipocytes accumulation combined with decreased synthesis of tendon-related proteins compared to 2 months old rats 136 which are close age to rats used in our study. Therefore, the effects of each treatment on elongation-induced calcaneal tendon injury were not clearly delineated between treatments in the present study. Further aspects of the present disclosure are provided by the subject matter of the following clauses.

A method of inducing tenogenic differentiation in a population of adipose-derived stem cells (ASCs) cultured in a bioreactor system, includes directly applying or infusing the population of ASCs onto a porous biopolymer-based scaffold to form a cell-scaffold construct, culturing the cell-scaffold construct in a medium for a period of time to produce a population of tenocyte-like cells expressing at least one tenogenic transcription factor, at least one tendon-specific extracellular matrix gene, or a combination thereof, and maturing said population of differentiated cells to produce a population of tenocyte-like cells expressing express at least one tendon marker gene. The step of culturing the cell-scaffold construct in a medium for a period of time to produce a population of tenocyte-like cells includes contacting the cell-scaffold construct with at least one tenogenic differentiation driver and applying controlled mechanical stimulations to the cell-scaffold construct. The controlled mechanical stimulations include flow shear stress, and dynamic or static tensile strain.

The method of the preceding clause, wherein the population of differentiated cells comprises a population of tenoblast-like cells.

A method of producing a tendon neotissue from a population of adipose-derived stem cells (ASCs) cultured in a bioreactor system, includes directly applying or infusing the population of ASCs onto a porous biopolymer-based scaffold to form a cell-scaffold construct, and culturing the cell-scaffold construct in a medium for a period of time to produce a population of tenocyte-like cells expressing at least one tendon marker gene and organized to form said tendon neotissue. The culturing the cell-scaffold construct in a medium for a period of time to produce a population of tenocyte-like cells includes contacting the cell-scaffold construct with at least one tenogenic differentiation driver and applying controlled mechanical stimulations to the cell-scaffold construct. The controlled mechanical stimulations applied to the cell scaffold construct includes flow shear stress, and dynamic or static tensile strain.

The method of the preceding clause, wherein the tendon neotissue further comprises a population of tenoblast-like cells.

The method of any preceding clause, wherein the porous biopolymer-based scaffold is ligated by a filament or net and shaped as a column along a longitudinal axis.

The method of any preceding clause, wherein the tendon neotissue comprising the population of tenocyte-like cells are embedded within a fibrous extracellular matrix (ECM) attached to the biopolymer-based scaffold and are organized parallel to each other along the biopolymer-based scaffold longitudinal axis.

The method of any preceding clause, wherein the population of tenocyte-like cells have an elongated rod-like nucleus and express ECM components.

The method of any preceding clause wherein said flow shear stress is induced by a perfusion flow and a centrifugal flow motion of the medium.

The method of any preceding clause, wherein the perfusion flow is bidirectional with a rate of between 2 and 50 ml/minute, preferably 10 ml/minute.

The method of any preceding clause, wherein the centrifugal flow motion is produced by agitating the medium at a speed of between 10 and 10,000 rpm, preferably 300 rpm.

The method of any preceding clause, wherein the dynamic or static tensile strain has an amplitude of between 1 and 75%, preferably 10%.

The method of any preceding clause, wherein the dynamic or static tensile strain is continuous.

The method of any preceding clause, wherein the porous biopolymer-based scaffold comprised at least 5% of collagen type I, preferably 80%.

The method of any preceding clause, wherein the biopolymer-based scaffold is infused with the population of ASCs, preferably at a density of between 5×10⁰ and 1.0×10⁹ ASCs/cm³, preferably 1.0×10⁶ ASCs/cm³.

The method of any preceding clause, wherein the period of time ranges between 3 and 180 days.

The method of any preceding clause, wherein the at least one tenogenic differentiation driver is a member of the TGF growth factor family, preferably TGF-β1.

The method of any preceding clause, wherein the at least one tendon marker gene is selected from the group of consisting of fibromodulin (Fbmd), collagen 14a1 (COL14a1), and truncated hemoglobin 4 (THBS4).

The method of any preceding clause, wherein the at least one tenogenic transcription factor gene is selected from the group consisting of scleraxis (Scx), mohawk (Mkx), early growth response 1 (Egr1), connective tissue growth factor (CTGF) and lysyl oxidase (LOX).

The method any preceding clause, wherein the at least one tendon-ECM gene is selected from the group consisting of collagen Ial (COL1a1), collagen 3a1 (COL3a1), decorin (Dcn), elastin (Eln), tenascin-C (TnC), and biglycan (Bgn).

An implant includes at least one tendon neotissue produced according to the method of any preceding clause. The implant is effective for use in the treatment of a tendon or ligament injury in a mammal subject.

The implant of any preceding clause, further comprising a molecule selected from the group consisting of collagen, laminin, fibronectin, PLA, PGLA, PLLA, PEEK, PEG, elastin, tenomodulin, fibromodulin, and combination thereof.

A method of treating a tendon or ligament injury includes the implantation of at least one tendon neotissue produced according to the method of any preceding clause, or at least one implant according to any preceding clause, in a mammal subject.

A bioreactor system for producing a tri-dimensional engineered tissue includes a bioreactor which includes a base chamber containing a medium and a construct comprising a biopolymer-based scaffold comprising a population of multipotent cells, a core frame, a top lid and a perfusion system. The core frame includes an immobile horizontal bar at the bottom for securing one extremity of the construct and a gas exchange access port. The top lid includes a medium access port, and an adjustable horizontal bar for securing the other extremity of the construct, the adjustable horizontal bar being attached to a vertical threaded bar that can move upward and downward, thereby allowing for a change in distance between the immobile horizontal bar and the adjustable horizontal bar to apply an adjustable dynamic or static tensile strain to the construct. The perfusion system includes a medium reservoir, a peristaltic pump and an agitator. The medium reservoir contains the medium, a deformable membrane for facilitating gas exchange with the base chamber, a medium access port, and a gas exchange access port. The peristaltic pump connects to the medium access port of the medium reservoir and to the medium access port of the top lid for controlling the rate and directionality of a flow of said medium in and/or out of the base chamber. The agitator to create centrifugal flow motion and enhance nutrients and gas diffusion.

The bioreactor system of the preceding clause, wherein the engineered tissue is a tendon neotissue.

The bioreactor system of any preceding clause, wherein the dynamic or static tensile strain applied to the construct has an amplitude of about 1 to 75%, preferably 10%. The bioreactor system of any preceding clause, wherein the dynamic or static tensile strain applied to the construct is continuous.

The bioreactor system of any preceding clause, wherein said perfusion system produces a bidirectional perfusion flow and a centrifugal flow motion, thereby inducing flow shear stress onto the construct. The bioreactor system of any preceding clause, wherein the rate of the perfusion flow is between 2 and 50 ml/minute, preferably 10 ml/minute.

The bioreactor system of any preceding clause, wherein the centrifugal flow motion is produced by the device applying agitation at a speed of between 10 and 10,000 rpm, preferably 300 rpm.

The perfusion bioreactor system of any preceding clause, wherein the perfusion system is maintained in a thermoregulated CO₂ incubator.

The bioreactor system of any preceding clause, wherein the biopolymer comprises collagen of type I and the population of multipotent cells is a population of ASCs.

The bioreactor system of any preceding clause, wherein the biopolymer-based scaffold is infused with the population of ASCs at a density of between 5.5×10⁰ and 1.0×10⁹ ASCs/cm³, preferably 1.0×10⁶ ASCs/cm³.

The bioreactor system of any preceding claim, wherein the flow of medium comprises at least one tenogenic differentiation driver.

We claim:

1. A method of inducing tenogenic differentiation in a population of adipose-derived stem cells (ASCs) cultured in a bioreactor system, comprising:
directly applying or infusing said population of ASCs onto a porous biopolymer-based scaffold to form a cell-scaffold construct,
culturing said cell-scaffold construct in a medium for a period of time to produce a population of tenocyte-like cells, comprising:
contacting said cell-scaffold construct with at least one tenogenic differentiation driver, and
applying controlled mechanical stimulations to said cell-scaffold construct comprising:
flow shear stress, and
dynamic or static tensile strain,
to produce a population of differentiated cells, expressing at least one tenogenic transcription factor, at least one tendon-specific extracellular matrix gene, or a combination thereof,
maturing said population of differentiated cells to produce a population of tenocyte-like cells,
wherein said population of tenocyte-like cells express at least one tendon marker gene.

2. The method of claim 1, wherein the population of differentiated cells comprises a population of tenoblast-like cells.

3. A method of producing a tendon neotissue from a population of ASCs cultured in a bioreactor system, comprising:
directly applying or infusing said population of ASCs onto a porous biopolymer-based scaffold to form a cell-scaffold construct,
culturing said cell-scaffold construct in a medium for a period of time to produce a population of tenocyte-like cells comprising:
contacting said cell-scaffold construct with at least one tenogenic differentiation driver, and
applying controlled mechanical stimulations to said cell-scaffold construct comprising:
flow shear stress, and
dynamic or static tensile strain,
wherein said population of tenocyte-like cells express at least one tendon marker gene and organize to form said tendon neotissue.

4. The method of claim 3, wherein the tendon neotissue further comprises a population of tenoblast-like cells.

5. The method of claim 1, wherein the porous biopolymer-based scaffold is ligated by a filament or net and shaped as a column along a longitudinal axis.

6. The method of claim 5, wherein the tendon neotissue comprising the population of tenocyte-like cells are embedded within a fibrous extracellular matrix (ECM) attached to the biopolymer-based scaffold and are organized parallel to each other along the biopolymer-based scaffold longitudinal axis, and wherein the population of tenocyte-like cells have an elongated rod-like nucleus and express ECM components.

7. The method of claim 1, wherein said flow shear stress is induced by a perfusion flow and a centrifugal flow motion of the medium.

8. The method of claim 7, wherein the perfusion flow is bidirectional with a rate of between 2 and 50 ml/minute and the centrifugal flow motion is produced by agitating the medium at a speed of between 10 and 10,000 rpm.

9. The method of claim 1, wherein the static tensile strain is continuous with an amplitude of between 1 and 75%.

10. The method of claim 1, wherein the porous biopolymer-based scaffold comprised at least 5% of collagen type I and is infused with the population of ASCs at a density of between 5×10⁰ and 1×10⁹ ASCs/cm³.

11. The method of claim 1, wherein the at least one tenogenic differentiation driver is a member of the TGF growth factor family.

12. The method of claim 1, wherein the at least one tendon marker gene is selected from the group of consisting of fibromodulin (Fbmd), collagen I4a1 (COL14a1), and truncated hemoglobin 4 (THBS4); wherein the at least one tenogenic transcription factor gene is selected from the group consisting of scleraxis (Scx), mohawk (Mkx), early growth response 1 (Egr1), connective tissue growth factor (CTGF) and lysyl oxidase (LOX), and, wherein the at least one tendon-ECM gene is selected from the group consisting of collagen Ia1 (COL1a1), collagen 3a1 (Col3a1), decorin (Dcn), elastin (Eln), tenascin-C (TnC), and biglycan (Bgn).

13. An implant for use in the treatment of a tendon or ligament injury in a mammal subject comprising at least one tendon neotissue produced according to the method of claim 3.

14. The implant of claim 13, further comprising a molecule selected from the group consisting of collagen, laminin, fibronectin, PLA, PGLA, PLLA, PEEK, PEG, elastin, tenomodulin, fibromodulin, and combination thereof.

15. A method of treating a tendon or ligament injury comprising the implantation of at least one implant according to claim 13, in a mammalian subject.

16. A bioreactor system for producing a tri-dimensional engineered tissue comprising:
the bioreactor comprising:
a base chamber containing a medium and a construct comprising a biopolymer-based scaffold comprising a population of multipotent cells,
a core frame comprising an immobile horizontal bar at the bottom for securing one extremity of said construct and a gas exchange access port,
a top lid comprising:
a medium access port, and
an adjustable horizontal bar for securing the other extremity of said construct, said adjustable horizontal bar being attached to a vertical threaded bar that can move upward and downward thereby allowing for a change in distance between said immobile horizontal bar and said adjustable horizontal bar to apply an adjustable dynamic or static tensile strain to said construct,
a perfusion system comprising:
a medium reservoir containing the medium, a deformable membrane for facilitating gas exchange with said base chamber, a medium access port, and a gas exchange access port,
a peristaltic pump connected to said medium access port of said medium reservoir and to said medium access port of said top lid for controlling the rate and directionality of a flow of said medium in and/or out of said base chamber,
an agitator to create centrifugal flow motion and enhance nutrients and gas diffusion.

17. The bioreactor system of claim 16, wherein the engineered tissue is a tendon neotissue.

18. The bioreactor system of claim 16, wherein the static tensile strain applied to the construct has an amplitude of about 1 to 75%, and wherein said perfusion system produces a bidirectional perfusion flow and a centrifugal flow motion, thereby inducing flow shear stress onto the construct.

19. The bioreactor system of claim 16, wherein the biopolymer-based scaffold comprises collagen of type I and is infused with the population of ASCs at a density of between 5×10⁰ and 1.0×10⁹ ASCs/cm³.

20. The bioreactor system of claim 16, wherein the flow of medium comprises at least one tenogenic differentiation driver.


SEQUENCE LISTING (42 sequences)

SEQ ID NO: 1 - DNA, 20 bp, synthetic construct
  source 1..20: mol_type = other DNA
tctgcctcagcaaccagaga

SEQ ID NO: 2 - DNA, 20 bp, synthetic construct
  source 1..20: mol_type = other DNA
aaagttccagtgggtctggg

SEQ ID NO: 3 - DNA, 20 bp, synthetic construct
  source 1..20: mol_type = other DNA
agtggctttacaagcaccgt

SEQ ID NO: 4 - DNA, 20 bp, synthetic construct
  source 1..20: mol_type = other DNA
acactaagccgctcagcatt

SEQ ID NO: 5 - DNA, 21 bp, synthetic construct
  source 1..21: mol_type = other DNA
cctacgagcacctgacctcag

SEQ ID NO: 6 - DNA, 22 bp, synthetic construct
  source 1..22: mol_type = other DNA
gatggtgctgaagatgaagtgg

SEQ ID NO: 7 - DNA, 20 bp, synthetic construct
  source 1..20: mol_type = other DNA
acccgcgttaccaatgacaa

SEQ ID NO: 8 - DNA, 20 bp, synthetic construct
  source 1..20: mol_type = other DNA
ggcttggagattttgggggt

SEQ ID NO: 9 - DNA, 20 bp, synthetic construct
  source 1..20: mol_type = other DNA
caggcgatttgcgtgtactg

SEQ ID NO: 10 - DNA, 20 bp, synthetic construct
  source 1..20: mol_type = other DNA
acttcagaacaccaggcact

SEQ ID NO: 11 - DNA, 20 bp, synthetic construct
  source 1..20: mol_type = other DNA
caagaggagggccaagaaga

SEQ ID NO: 12 - DNA, 20 bp, synthetic construct
  source 1..20: mol_type = other DNA
tcctgtggtttggtcgtctg

SEQ ID NO: 13 - DNA, 20 bp, synthetic construct
  source 1..20: mol_type = other DNA
tcctggggctagtggtagtc

SEQ ID NO: 14 - DNA, 20 bp, synthetic construct
  source 1..20: mol_type = other DNA
ggcgaaccatctttgccatc

SEQ ID NO: 15 - DNA, 18 bp, synthetic construct
  source 1..18: mol_type = other DNA
ttatcaaagtgcctggtg

SEQ ID NO: 16 - DNA, 18 bp, synthetic construct
  source 1..18: mol_type = other DNA
catagacacatcggaagg

SEQ ID NO: 17 - DNA, 20 bp, synthetic construct
  source 1..20: mol_type = other DNA
ctatggtgtcggtgtcggag

SEQ ID NO: 18 - DNA, 20 bp, synthetic construct
  source 1..20: mol_type = other DNA
gggggctaacccaaactgag

SEQ ID NO: 19 - DNA, 20 bp, synthetic construct
  source 1..20: mol_type = other DNA
tactgatggggccttcgaga

SEQ ID NO: 20 - DNA, 20 bp, synthetic construct
  source 1..20: mol_type = other DNA
agcagcttcccagaatccac

SEQ ID NO: 21 - DNA, 22 bp, synthetic construct
  source 1..22: mol_type = other DNA
tgattgagaacgggagcctgag

SEQ ID NO: 22 - DNA, 22 bp, synthetic construct
  source 1..22: mol_type = other DNA
tttggtgatgttgttggtgtgc

SEQ ID NO: 23 - DNA, 18 bp, synthetic construct
  source 1..18: mol_type = other DNA
gcttctgctgagggacac

SEQ ID NO: 24 - DNA, 18 bp, synthetic construct
  source 1..18: mol_type = other DNA
gatttctggggttgggac

SEQ ID NO: 25 - DNA, 18 bp, synthetic construct
  source 1..18: mol_type = other DNA
ctggacgatggaagtgag

SEQ ID NO: 26 - DNA, 18 bp, synthetic construct
  source 1..18: mol_type = other DNA
gtgaccctgaactgctgc

SEQ ID NO: 27 - DNA, 20 bp, synthetic construct
  source 1..20: mol_type = other DNA
acgtaaacacccagacggac

SEQ ID NO: 28 - DNA, 20 bp, synthetic construct
  source 1..20: mol_type = other DNA
caccaactcggagccttcat

SEQ ID NO: 29 - DNA, 18 bp, synthetic construct
  source 1..18: mol_type = other DNA
gtgtccccacccctaacg

SEQ ID NO: 30 - DNA, 18 bp, synthetic construct
  source 1..18: mol_type = other DNA
agtgtagcccaggatgcc

SEQ ID NO: 31 - DNA, 20 bp, synthetic construct
  source 1..20: mol_type = other DNA
ggagagagtgccaactccag

SEQ ID NO: 32 - DNA, 20 bp, synthetic construct
  source 1..20: mol_type = other DNA
gtgctttggaaaatggtgct

SEQ ID NO: 33 - DNA, 22 bp, synthetic construct
  source 1..22: mol_type = other DNA
tcccagaacattacataccact

SEQ ID NO: 34 - DNA, 20 bp, synthetic construct
  source 1..20: mol_type = other DNA
gctatttccttcagccttga

SEQ ID NO: 35 - DNA, 22 bp, synthetic construct
  source 1..22: mol_type = other DNA
agatgctactccagacggtttc

SEQ ID NO: 36 - DNA, 23 bp, synthetic construct
  source 1..23: mol_type = other DNA
cacggcttattccatagagttca

SEQ ID NO: 37 - DNA, 21 bp, synthetic construct
  source 1..21: mol_type = other DNA
aacacggccttcactgcgctg

SEQ ID NO: 38 - DNA, 21 bp, synthetic construct
  source 1..21: mol_type = other DNA
cagtagcacgttgcccaggtg

SEQ ID NO: 39 - DNA, 20 bp, synthetic construct
  source 1..20: mol_type = other DNA
gtggtcccacaagtgaaggt

SEQ ID NO: 40 - DNA, 20 bp, synthetic construct
  source 1..20: mol_type = other DNA
gtcttcctcgcttgcttgtc

SEQ ID NO: 41 - DNA, 22 bp, synthetic construct
  source 1..22: mol_type = other DNA
aagttcaacggcacagtcaagg

SEQ ID NO: 42 - DNA, 22 bp, synthetic construct
  source 1..22: mol_type = other DNA
cgccagtagactccacgacata